(19) United States
(12) Patent Application Publication
(10) Pub. No.: US 2026/0355189 A1
(43) Pub. Date: Nov. 12, 2026

(54) SMALL MOLECULE INHIBITORS OF NEUTROPHIL EXOCYTOSIS AND INFLAMMATION

(71) Applicant: THE SCRIPPS RESEARCH INSTITUTE, La Jolla, CA (US)

(72) Inventors: Sergio D. CATZ, San Diego, CA (US); Jennifer L. JOHNSON, San Diego, CA (US)

(21) Appl. No.: 19/113,167

(22) PCT Filed: Sep. 21, 2023
(86) PCT No.: PCT/US2023/074722

§ 371 Date: Mar. 19, 2025

Related U.S. Application Data
(60) Provisional application No. 63/376,685, filed on Sep. 22, 2022.

(51) Int. Cl. A61K 31/502; A61K 31/4196; A61K 31/422; A61K 31/423; A61K 31/427; A61K 31/4365; A61K 31/437; A61K 31/438; A61K 31/4439; A61K 31/454; A61K 31/4545; A61K 31/46; A61K 31/4709; A61K 31/4725; A61K 31/4745; A61K 31/496; A61K 31/501; A61K 31/506; A61K 31/5377; A61K 31/541; A61K 31/551; A61P 29/00; C07D 249/08; C07D 401/12; C07D 401/14; C07D 403/12; C07D 405/12; C07D 409/12; C07D 409/14; C07D 413/12; C07D 417/12; C07D 451/02; C07D 471/04; C07D 471/16; C07D 471/20; C07D 487/04; C07D 495/04

(52) U.S. Cl. CPC A61K 31/502; A61K 31/4196; A61K 31/422; A61K 31/423; A61K 31/427; A61K 31/4365; A61K 31/437; A61K 31/438; A61K 31/4439; A61K 31/454; A61K 31/4545; A61K 31/46; A61K 31/4709; A61K 31/4725; A61K 31/4745; A61K 31/496; A61K 31/501; A61K 31/506; A61K 31/5377; A61K 31/541; A61K 31/551; A61P 29/00; C07D 249/08; C07D 401/12; C07D 401/14; C07D 403/12; C07D 405/12; C07D 409/12; C07D 409/14; C07D 413/12; C07D 417/12; C07D 451/02; C07D 471/04; C07D 471/16; C07D 471/20; C07D 487/04; C07D 495/04

(57) ABSTRACT

The present disclosure provides a compound of Formula (I): or pharmaceutically acceptable salt thereof, wherein R1, X, and n are defined herein, their pharmaceutical compositions, and methods of use for treating diseases that are vulnerable to inhibition neutrophil exocytosis, wherein the disease etiology derives from neutrophil-mediated inflammation, upregulation of activation signature molecules in neutrophils, or increased tissue infiltration by inflammatory neutrophils.

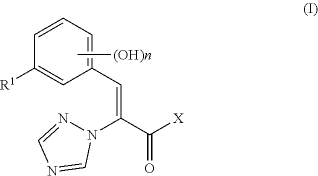

### CROSS REFERENCE TO RELATED APPLICATION

This application claims priority to U.S. provisional patent application No. 63/376,685, which was filed on Sep. 22, 2022, and which is hereby incorporated by reference in its entirety.

### STATEMENT OF GOVERNMENT SUPPORT

This invention was made with government support under HL088256 awarded by the National Institutes of Health. The government has certain rights in the invention.

### BACKGROUND

Neutrophils constitute the first line of cellular defense in response to bacterial and fungal infections (1) and rely on granular proteins to kill microorganisms. However, uncontrolled secretion of granular proteins by neutrophils is injurious to the host. Increased plasma levels of neutrophil secretory proteins, including myeloperoxidase (MPO) and elastase, are associated with tissue damage and are hallmarks of endotoxemia and sepsis and are also observed in sterile trauma, leading to systemic inflammatory response syndrome. Neutrophil-derived plasma MPO predicts endothelial dysfunction (2) and is an indicator of the onset of sepsis (3). MPO is also involved in the pathogenesis of cardiovascular disease and arthritis (4), (5). Similarly, secreted neutrophil elastase is implicated in the development of acute respiratory distress syndrome (6) and tissue damage through the release of proteolytic enzymes and oxygen radicals are involved in the development of sepsis (7), (8).

Neutrophils contain four secretory organelles that engage sequentially in exocytosis depending on stimuli strength (9), (10). At a site of infection where high concentrations of pathogens are present, neutrophil secrete specific and azurophilic granules, which contain the most toxic neutrophil cargos. However, neutrophils under pathological conditions encounter a variety of stimuli capable of inducing exocytosis in circulation, resulting in high levels of neutrophil secreted proteases and pro-oxidative factors that are associated with systemic inflammation.

### SUMMARY

To address uncontrolled neutrophil secretion that represents a major health hazard and provide a treatment for systemic inflammation mediated by neutrophil dysfunction, the present disclosure provides small molecule compounds that are potent inhibitors (“Inhibitors”) of the interaction between the small GTPase Rab27a and its effector JFC1, which are two central regulators of neutrophil exocytosis. In various embodiments, the present disclosure provides Inhibitors, or pharmaceutically acceptable salts thereof, that are of Formula (I):

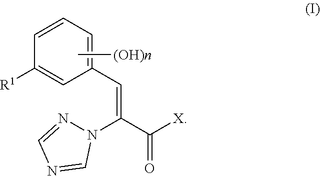

R¹ is selected from the group consisting of H, —NO₂, —OH, —C₁-C₆-alkyl, —C₁-C₆-haloalkyl, —C₁-C₆-alkyl-O—(C₁-C₆-alkyl), —C₁-C₆-alkenyl, —C₁-C₆-alkoxyl, —NRR′, —C(O)R, C₃-C₈-cycloalkyl, and —C₆-C₁₀-aryl. In R¹, any alkyl, alkenyl, alkoxyl, and cycloalkyl is optionally substituted with 1 to 6 halo.
X is —NR^{X}(R²) or C₁-C₆-alkyl.
R^{X} is H or C₁-C₆-alkyl.
R² is selected from the group consisting of:
Het, Cy,
—C(O)(Het), —C(O)(Cy),
—C(O)(NHI)(Het), —C(O)(NHI)(Cy),
—OC(O)(Het), —OC(O)(Cy),
—O(Het), —O(Cy),
—(C₁-C₆-alkyl)(Het), —(C₁-C₆-alkyl)(Cy),
—(C₁-C₆-hydroxyalkyl)(Het), —(C₁-C₆-hydroxyalkyl)(Cy),
—(C₂-C₆-alkenyl)(Het), —(C₂-C₆-alkenyl)(Cy),
—(NR)(C₁-C₆-alkyl)(Het), —(NR)(C₁-C₆-alkyl)(Cy),
—(NR)C(O)(Het), —(NR)C(O)(Cy),
—(C₃-C₈-cycloalkyl)N(R)C(O)—Cy,
—C₁-C₆-alkyl(NR)C(O)N(C₆-C₁₀-aryl)₂, and
—C₁-C₆-alkyl (optionally substituted with NRR′).

Het is a 3- to 20-membered monocyclic, bicyclic, tricyclic, or tetracyclic heterocyclic ring system wherein 1 to 6 ring members are independently selected from N, O, and S, and wherein the ring system is fully saturated, partially saturated, aromatic, or a combination thereof.

Cy is a 4- to 15-membered monocyclic, bicyclic, or tricyclic carbocyclic ring system that is fully saturated, partially saturated, aromatic, or a combination thereof.

Het and Cy are optionally substituted with 1 to 6 substituents independently selected from the group consisting of halo, —OH, —CN, oxo, thio, —NRR′, —C₁-C₆-alkyl, —C₁-C₆-haloalkyl, —C₃-C₈-cycloalkyl, —(C₁-C₆-alkyl)(NH)₀₋₁(C₃-C₈-cycloalkyl), —C₆-C₁₀-aryl, —O—C₆-C₁₀-aryl, —C(O)—C₆-C₁₀-aryl, —C(O)NH—C₆-C₁₀-aryl, —S(O)₀₋₂—C₆-C₁₀-aryl, —(C₁-C₆-alkyl)-(O)₀₋₁—(C₆-C₁₀-aryl), —C(O)NRR′, —C(O)OR, 5- to 10-membered heteroaryl (wherein 1-4 heteroaryl members are independently selected from N, O, and S), —C(O)(5- to 10-membered heteroaryl (wherein 1-4 heteroaryl members are independently selected from N, O, and S)), —NR(C₁-C₆-alkyl)(5- to 10-membered heteroaryl (wherein 1-4 heteroaryl members are independently selected from N, O, and S), 5- to 8-membered heterocycloalkyl (wherein 1-4 ring members are independently selected from N, O, and S) optionally fused to C₆-C₁₀-aryl, —(C₁-C₆-alkyl)(CONH)₀₋₁(5- to 8-membered heterocycloalkyl (wherein 1-4 ring members are independently selected from N, O, and S)). Any alkyl, cycloalkyl, heterocycloalkyl, aryl, or heteroaryl moiety in Het or Cy is substituted by 1 to 3 substituents selected from the group consisting of halo, —CN, oxo, —OH, —NRR′, —C₁-C₆-alkyl, —C₁-C₆-haloalkyl, and —(C₁-C₆-alkyl)(C₆-C₁₀-aryl).

Each instance of R and R′ is independently selected from H and C₁-C₆-alkyl.

In some embodiments, R and R′, together with the nitrogen atom to which they are bound, form a 5- to 8-membered heterocycloalkyl (wherein 1-3 additional ring members are independently selected from N, O, and S).

Subscript n is 0 or 1.

The definitions above notwithstanding, Formula (I) does not include the following compound:

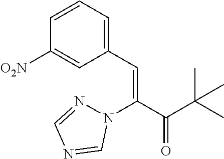 [SMILES: CC(C)(C)C(=O)/C(=C/c1cccc([N+](=O)[O-])c1)n1cncn1]

The present disclosure also provides in embodiments a pharmaceutical composition comprising one or more Inhibitors or a pharmaceutically acceptable salt thereof as described herein and a pharmaceutically acceptable carrier.

In additional embodiments, the present disclosure provides a method for treating a disease in a subject suffering therefrom, wherein the disease is one selected from those described herein. The method comprises administering to the subject one or more Inhibitors or pharmaceutically acceptable salt thereof as described herein.

### BRIEF DESCRIPTION OF THE DRAWINGS

FIGS. 1A-B illustrates the cellular activities of the Inhibitors. FIG. 1A illustrates the mechanism of exocytosis of a neutrophil granule's cargo. FIG. 1B illustrates the point of inhibition of granule exocytosis by the Inhibitors.

FIGS. 2A-G are schematics of a molecular docketing analysis of the Inhibitors on Rab27a. FIG. 2A is a three-dimensional model of the Inhibitor Nexinhib 20 bound inside a binding pocket of Rab27a. FIG. 2B illustrates the specific Rab27a amino acid interactions with Nexinhib 20. FIG. 2C illustrates the specific Rab27a amino acid interactions with the Inhibitor C₁. FIG. 2D illustrates the specific Rab27a amino acid interactions with the Inhibitor C2. FIG. 2E illustrates the specific Rab27a amino acid interactions with the Inhibitor C4. FIG. 2F illustrates the specific Rab27a amino acid interactions with the Inhibitor C5. FIG. 2G illustrates the specific Rab27a amino acid interactions with the Inhibitor C6.

FIGS. 3A-C illustrate the effect of the Inhibitors of Rab27a on MPO secretion of mouse and human neutrophils. FIG. 3A is a schematic representation of a cell-based chemiluminescence-based exocytosis assay used for the identification of cell-active Inhibitors of MPO secretion. FIG. 3B is a graph showing the effect of the Inhibitors of Rab27a on MPO secretion in purified mouse neutrophils. FIG. 3C is a graph of a dose-response analysis of the Inhibitor C1 using the chemiluminescence-based exocytosis assay.

FIG. 4 is a flow cytometry analysis of the effect of the Inhibitors on the mobilization of azurophilic granules in human neutrophils.

FIG. 5 is a flow cytometry analysis of the effect of the Inhibitors on the exocytosis of specific granules in human neutrophils.

FIG. 6 is a graph showing the effect of the Inhibitors on the exocytosis of Gelatinase granule cargo MMP9 in human neutrophils.

FIG. 7 is a graph showing the effect of the Inhibitors on the exocytosis of secretory vesicles and the upregulation of the adhesion molecule CD11b at the plasma membrane in human neutrophils.

FIG. 8A-C. New neutrophil exocytosis inhibitors (NEIa-) C1, C4 and C6 inhibit the hyperactivation of secretion induced by the Nlrp3A350V mutation (purple, •), compared to wild-type controls (black symbols). In this model, Gasdermin D is inactive and so, exacerbated secretion is mediated by the Nlrp3 activation in a neutrophil intrinsic manner. A, gelatinase (MMP9) granules; B, azurophilic granules (MPO, myeloperoxidase); C, CD63 (an endolysosomal marker). In these experiments, the effective inhibition of neutrophil exocytosis was demonstrated using Nexinhib analogs at 10 μM or vehicle (DMSO) to treat bone-marrow-derived neutrophils for 1 hour. Subsequently, neutrophils were either stimulated with lipopolisacharide (LPS) and the chemotatctic peptide fMLF or left untreated. C20 (Nexinhib20 is used as control). Mean±SEM, n=3. In this and subsequent figures, p<0.05; **, p<0.01; ***, p<0.001; ****, p<0.0001, ANOVA.

FIG. 9A-F. A) Compound NEIa-C₆ (C₆) inhibits neutrophil mediated inflammation in vivo, in a model of cryopyrin-associated periodic syndrome. Nlrp3^{A355V} cre ERT2 and control mice were injected i.p. with 50 mg/kg tamoxifen followed by C₆ (30 mg/Kg) or vehicle as described. B) Total leukocytes numbers and subtypes were not affected. C) Animals suffer weight loss with disease progression. D) The activation of circulating neutrophils is attenuated by C6. E, The percentage of immature (pro-inflammatory, Ly6G-) neutrophils in circulation (blood) in Nlrp3^{A350V} is attenuated by treatment with C6 (4.29%) as compared to vehicle (11.0%). F, Neutrophilic infiltration into the kidney measured as total MMP9 and MPO levels is significantly decreased in C₆ treated mice. F, The protection was specific for kidneys.

FIG. 10A-B. Treatment with compound NEIa-C6 inhibits pro-inflammatory cytokine production in a model of sepsis. We tested the ability of C6 to protect mice from LPS-induced systemic inflammation. In these experiments, mice (n=4 independent mice per group) receive NEIa-C6 (30 mg/Kg) or vehicle by a single i.p. injection and two hours later, the animals are challenged with a single i.p. injection of lipopolysaccharide (LPS, 8 mg/Kg) or PBS (vehicle for LPS). Four hours after LPS/PBS insult, blood is collected and cytokines are analyzed in plasma using multiplex technology. A, C₆ inhibit the production of the indicated pro-inflammatory cytokines. B, C₆ treatment increases the production of IL-10, one of the most important anti-inflammatory cytokine.

FIG. 11A-B. A, Docking analysis of NEI20 compound reveals pockets in Rab27a (green) for potential substitution and extension of the selected hits. B, Structures and scores of NEI20 and NEIa-C6 analog.

FIG. 12. Structure and scores of newly designed analogs with modifications and substitutions in C3 or C10.

### DETAILED DESCRIPTION

The Inhibitors of the present disclosure are potent small molecule inhibitors of neutrophil exocytosis of specific and azurophilic granules, and are therefore useful, in various embodiments, as therapeutics for treating inflammatory processes including sepsis, arthritis, cardiovascular disease, acute lung injury, glomerulonephritis, autoimmune disorders and cancer. The present disclosure satisfies needs in the art for therapies that reduce high levels of neutrophil proteases and pro-oxidative secretions associated with systemic inflammation, but without affecting other important innate immune responses, including phagocytosis and neutrophil extracellular trap (NET) production. Compounds of the present disclosure target the interaction between the small GTPase Rab27a and its effector JFC1, two central regulators of neutrophil exocytosis which fulfills the desired activities of inhibiting exocytosis of azurophilic and specific granules and superoxide production without impairing other innate immune responses of neutrophils. The compounds are therefore of robust utility in treating various diseases associated with systemic neutrophil-dependent inflammation.

The compounds engender numerous advantages. When used in vivo, the Inhibitors of the present disclosure decrease neutrophil secretion of their most toxic granules without affecting other aspects of the neutrophil innate immune response. In addition, the Inhibitors decrease neutrophil infiltration into tissues in vivo, supporting the use of these compounds as systemic anti-inflammatory agents in many neutrophil-mediated pathological processes including coronary artery disease, autoinflammatory disease, sepsis, arthritis, ischemia-reperfusion injury, acute lung injury, glomerulonephritis, autoimmune disorders and cancer.

### Rab27a-JFC1

The interaction between Rab27a and JFC1 proteins regulates the intracellular trafficking, docking, fusion, and exocytosis of neutrophil specific and azurophilic granules which are the most toxic neutrophil cargoes. Rab27a and JFC1 proteins are dispensable for other innate immune-related neutrophil functions, including phagocytosis (11) and NET production (12). Exocytosis of specific and azurophilic granules is dependent on JFC1 binding to Rab27a because mutation of tryptophan 83 of the TGDWF domain of JFC1 abolishes JFC1-Rab27a binding and inhibits secretion (13). JFC1 is one of eleven Rab27a effectors, and it is essential in neutrophils but dispensable for other cells. By targeting Rab27a-JFC1 interaction, the Inhibitors of the present disclosure are specific for JFC1 (neutrophils).

Rab27a and its effector molecules play fundamental roles in the modulation of the neutrophil inflammatory response by controlling cellular release of inflammatory proteinases and oxidative factors, including myeloperoxidase (14, 15). Importantly, knocking out either Rab27a or JFC1 decreases plasma levels of neutrophil secretory proteins, reduces tissue infiltration by neutrophils, and increases survival in a mouse model of endotoxin-induced systemic inflammation (16). Because neither JFC1 nor Rab27a regulate trafficking of azurophilic granules to the phagosome (17, 11), the Inhibitors interfering with the Rab27a-JFC1 binding have similar anti-inflammatory properties without affecting other neutrophil innate immune functions.

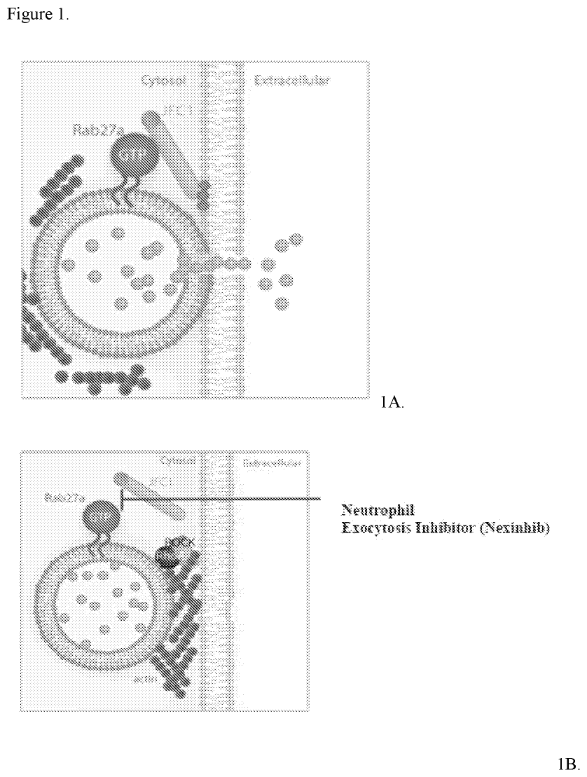

Referring to FIG. 1A, the inhibition of neutrophil exocytosis of specific and azurophilic granules by the Inhibitors disclosed herein is illustrated. Exocytosis occurs by recruitment of a granule from the neutrophil cytoplasm to the cell membrane, which is dependent on actin cytoskeleton remodeling and microtubule assembly. This is followed by granule tethering and docking to the cell membrane, leading to contact of the outer surface of the lipid bilayer membrane surrounding the granule with the inner surface of the target membrane. Granule priming then follows to make granules fusion-competent to ensure that they fuse with the target membrane rapidly, and a reversible fusion pore structure develops between the granule and the target membrane. Granule fusion occurs by the expansion of the fusion pore, leading to complete fusion of the granule with the target membrane to release granular contents.

In vivo studies showed that the small GTPase Rab27a regulates azurophilic granule exocytosis (15). Using mouse neutrophils deficient in Rab27a (Rab27a^{ash/ash}), these knockout neutrophils have a decreased number of azurophilic granules near the plasma membrane and impaired azurophilic granule exocytosis. Exocytosis of secretory vesicles in Rab27-deficient neutrophils was functional, indicating that Rab27 GTPases selectively control the exocytosis of neutrophil granules. Rab27a regulates exocytosis through interaction with specific effector molecules, including JFC1. JFC1 regulates docking of secretory granules by bridging the Rab27a-containing vesicles and the plasma membrane through interaction with both Rab27a and with plasma membrane phosphatidylinositol 1,4,5-trisphosphate. Referring to FIG. 1B, the Inhibitors disclosed herein inhibit Rab27a-JFC1 binding and neutrophil exocytosis.

### DEFINITIONS

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O— neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

“Cargo” refers to the microbicidal and cytotoxic contents of neutrophil granules including myeloperoxidase (MPO), elastase, lactoferrin, and matrix metalloproteinases.

“Exocytosis,” also known as degranulation in neutrophils, refers to the regulated release at the cell surface of pre-formed cytotoxic proteins from neutrophil granules, which can include myeloperoxidase (MPO), elastase, lactoferrin, and matrix metalloproteinases, which possess potent antimicrobial activity but are also highly cytotoxic.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. Polycyclic heterocycloalkyl includes rings that can be fused, bridged, and/or spiro-fused. In addition, a heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

“Mobilization” refers to migration of neutrophil granule organelles to the cell membrane upon stimulation.

The term “nitrile” or “cyano” can be used interchangeably and refers to a —CN group.

A “hydroxyl” or “hydroxy” refers to an —OH group.

The Inhibitors described herein can exist in various isomeric forms, including configurational, geometric, and conformational isomers, including, for example, cis- or trans-conformations. The Inhibitors may also exist in one or more tautomeric forms, including both single tautomers and mixtures of tautomers. The term “isomer” is intended to encompass all isomeric forms of the Inhibitors of this disclosure, including tautomeric forms of the Inhibitors. The Inhibitors of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the Inhibitors may exist primarily in an open-chained form under acidic conditions but cyclize under neutral conditions. All forms are included in the disclosure.

Some Inhibitors described herein can have asymmetric centers and therefore exist in different enantiomeric and diastereomeric forms. An Inhibitor as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the Inhibitors of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of an Inhibitor that is substantially free of other stereoisomers of that compound. Thus, a stereomerically pure Inhibitor having one chiral center will be substantially free of the opposite enantiomer of the Inhibitor. A stereomerically pure Inhibitor having two chiral centers will be substantially free of other diastereomers of the Inhibitor. A typical stereomerically pure Inhibitor comprises greater than about 80% by weight of one stereoisomer of the Inhibitor and less than about 20% by weight of other stereoisomers of the Inhibitor, for example greater than about 90% by weight of one stereoisomer of the Inhibitor and less than about 10% by weight of the other stereoisomers of the Inhibitor, or greater than about 95% by weight of one stereoisomer of the Inhibitor and less than about 5% by weight of the other stereoisomers of the Inhibitor, or greater than about 97% by weight of one stereoisomer of the Inhibitor and less than about 3% by weight of the other stereoisomers of the Inhibitor, or greater than about 99% by weight of one stereoisomer of the Inhibitor and less than about 1% by weight of the other stereoisomers of the Inhibitor. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the Inhibitors are prepared as single enantiomers from the methods used to prepare them.

As used herein, and unless otherwise specified to the contrary, the term “Inhibitor” is inclusive in that it encompasses an Inhibitor or a pharmaceutically acceptable salt, stereoisomer, and/or tautomer thereof. Thus, for instance, an Inhibitors of Formula I includes a pharmaceutically acceptable salt of a tautomer of the Inhibitor.

In this disclosure, a “pharmaceutically acceptable salt” is a pharmaceutically acceptable, organic or inorganic acid or base salt of an Inhibitor described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic Inhibitors described herein to a patient with such a disease.

The terms “prevent,” “preventing,” and “prevention” refer to the prevention of the onset, recurrence, or spread of the disease in a patient resulting from the administration of one or more Inhibitors described herein.

The term “effective amount” refers to an amount of an Inhibitor as described herein or other active ingredient sufficient to provide a therapeutic or prophylactic benefit in the treatment or prevention of a disease or to delay or minimize symptoms associated with a disease. Further, a therapeutically effective amount with respect to an Inhibitor as described herein means that amount of therapeutic agent alone, or in combination with other therapies, that provides a therapeutic benefit in the treatment or prevention of a disease. Used in connection with an Inhibitor as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Inhibitors

In various embodiments, the present disclosure provides an Inhibitor of Formula (I), a tautomer, or a pharmaceutically acceptable salt thereof.

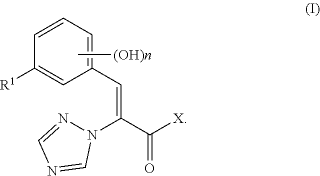

R¹ is selected from the group consisting of H, —NO₂, —OH, —C₁-C₆-alkyl, —C₁-C₆-haloalkyl, —C₁-C₆-alkyl-O—(C₁-C₆-alkyl), —C₁-C₆-alkenyl, —C₁-C₆-alkoxyl, —NRR′, —C(O)R, C₃-C₈-cycloalkyl, and —C₆-C₁₀-aryl. In R¹, any alkyl, alkenyl, alkoxyl, and cycloalkyl is optionally substituted with 1 to 6 halo.
X is —NR^{X}(R²) or C₁-C₆-alkyl.
R^{X} is H or C₁-C₆-alkyl.
R² is selected from the group consisting of:
Het, Cy,
—C(O)(Het), —C(O)(Cy),
—C(O)(NH)(Het), —C(O)(NH)(Cy),
—OC(O)(Het), —OC(O)(Cy),
—O(Het), —O(Cy),
—(C₁-C₆-alkyl)(Het), —(C₁-C₆-alkyl)(Cy),
—(C₁-C₆-hydroxyalkyl)(Het), —(C₁-C₆-hydroxyalkyl)(Cy),
—(C₂-C₆-alkenyl)(Het), —(C₂-C₆-alkenyl)(Cy),
—(NR)(C₁-C₆-alkyl)(Het), —(NR)(C₁-C₆-alkyl)(Cy),
—(NR)C(O)(Het), —(NR)C(O)(Cy),
—(C₃-C₈-cycloalkyl)N(R)C(O)—Cy,
—C₁-C₆-alkyl(NR)C(O)N(C₆-C₁₀-aryl)₂, and
—C₁-C₆-alkyl (optionally substituted with NRR′).

Het is a 3- to 20-membered monocyclic, bicyclic, tricyclic, or tetracyclic heterocyclic ring system wherein 1 to 6 ring members are independently selected from N, O, and S, and wherein the ring system is fully saturated, partially saturated, aromatic, or a combination thereof.

Cy is a 4- to 15-membered monocyclic, bicyclic, or tricyclic carbocyclic ring system that is fully saturated, partially saturated, aromatic, or a combination thereof.

Het and Cy are optionally substituted with 1 to 6 substituents independently selected from the group consisting of halo, —OH, —CN, oxo, thio, —NRR′, —C₁-C₆-alkyl, —C₁-C₆-haloalkyl, —C₃-C₈-cycloalkyl, —(C₁-C₆-alkyl)(NH)₀₋₁(C₃-C₈-cycloalkyl), —C₆-C₁₀-aryl, —O—C₆-C₁₀-aryl, —C(O)—C₆-C₁₀-aryl, —C(O)NH—C₆-C₁₀-aryl, —S(O)₀₋₂—C₆-C₁₀-aryl, —(C₁-C₆-alkyl)-(O)₀₋₁—(C₆-C₁₀-aryl), —C(O)NRR′, —C(O)OR, 5- to 10-membered heteroaryl (wherein 1-4 heteroaryl members are independently selected from N, O, and S), —C(O)(5- to 10-membered heteroaryl (wherein 1-4 heteroaryl members are independently selected from N, O, and S)), —NR(C₁-C₆-alkyl)(5- to 10-membered heteroaryl (wherein 1-4 heteroaryl members are independently selected from N, O, and S), 5- to 8-membered heterocycloalkyl (wherein 1-4 ring members are independently selected from N, O, and S) optionally fused to C₆-C₁₀-aryl, —(C₁-C₆-alkyl)(CONH)₀₋₁(5- to 8-membered heterocycloalkyl (wherein 1-4 ring members are independently selected from N, O, and S)). Any alkyl, cycloalkyl, heterocycloalkyl, aryl, or heteroaryl moiety in Het or Cy is substituted by 1 to 3 substituents selected from the group consisting of halo, —CN, oxo, —OH, —NRR′, —C₁-C₆-alkyl, —C₁-C₆-haloalkyl, and —(C₁-C₆-alkyl)(C₆-C₁₀-aryl).

Each instance of R and R′ is independently selected from H and C₁-C₆-alkyl.

In some embodiments, R and R′, together with the nitrogen atom to which they are bound, form a 5- to 8-membered heterocycloalkyl (wherein 1-3 additional ring members are independently selected from N, O, and S).

Subscript n is 0 or 1.

It should be understood that, the definitions above notwithstanding, Formula (I) does not include the compound below:

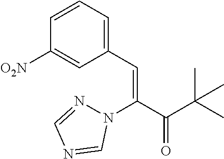 [SMILES: CC(C)(C)C(=O)/C(=C/c1cccc([N+](=O)[O-])c1)n1cncn1]

In some embodiments, R¹ is —NO₂.

In additional embodiments, n is 0. In other embodiments, n is 1.

In still additional embodiments, X is —NR^{X}(R²). In illustrative embodiments, R^{X} is H.

In various embodiments, R² is selected from the group consisting of —(NR)(C₁-C₆-alkyl)(Het), —(NR)(C₁-C₆-alkyl)(Cy), Cy, and —C₁-C₆-alkyl (optionally substituted with NRR′). Optionally in combination with this or any other embodiment described herein, Cy is a 6- to 12-membered monocyclic or bicyclic carbocyclic ring system. For example, Cy can be —C₆-C₁₀-aryl, such as phenyl or naphthyl. Alternatively, Het is a 5- to 12-membered monocyclic or bicyclic heterocyclic ring system.

The present disclosure also provides in various embodiments an Inhibitor or a pharmaceutically acceptable salt thereof, as set forth in Table 1.

TABLE 1
Exemplary Inhibitors

| | | Binding |
|---|---|---|
| Inhibitor | | Affinity(kcal/ |
| Number | Structure | mol) |
| 1 | 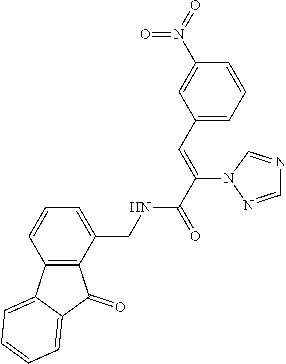 [SMILES: O=C(NCc1cccc2c1C(=O)c1ccccc1-2)/C(=C/c1cccc([N+](=O)[O-])c1)n1cncn1] | −7.9 |
| 2 | 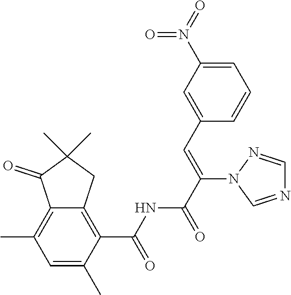 [SMILES: Cc1cc(C)c2c(c1C(=O)NC(=O)/C(=C/c1cccc([N+](=O)[O-])c1)n1cncn1)CC(C)(C)C2=O] | −7.6 |
| 3 | 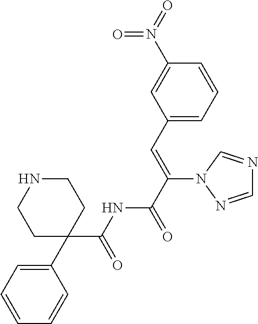 [SMILES: O=C(NC(=O)C1(c2ccccc2)CCNCC1)/C(=C/c1cccc([N+](=O)[O-])c1)n1cncn1] | −7.5 |
| 4 | 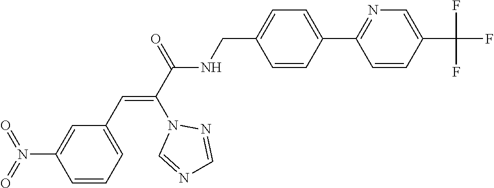 [SMILES: O=C(NCc1ccc(-c2ccc(C(F)(F)F)cn2)cc1)/C(=C/c1cccc([N+](=O)[O-])c1)n1cncn1] | −7.5 |
| 5 | 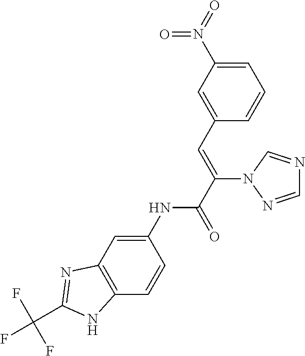 [SMILES: O=C(Nc1ccc2[nH]c(C(F)(F)F)nc2c1)/C(=C/c1cccc([N+](=O)[O-])c1)n1cncn1] | −7.5 |
| 6 | 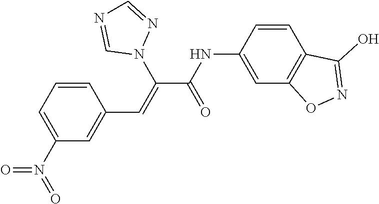 [SMILES: O=C(Nc1ccc2c(O)noc2c1)/C(=C/c1cccc([N+](=O)[O-])c1)n1cncn1] | −7.4 |
| 7 | 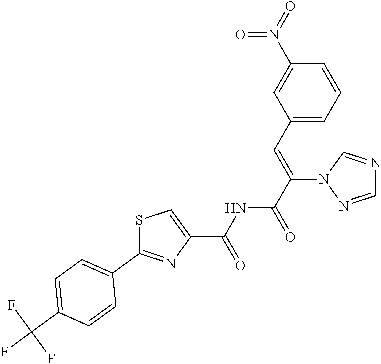 [SMILES: O=C(NC(=O)c1csc(-c2ccc(C(F)(F)F)cc2)n1)/C(=C/c1cccc([N+](=O)[O-])c1)n1cncn1] | −7.4 |
| 8 | 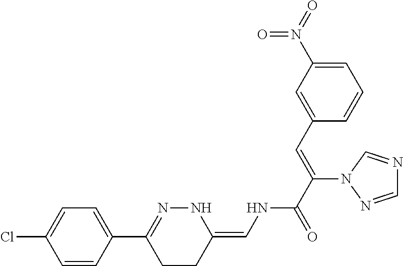 [SMILES: O=C(N/C=C1/CCC(c2ccc(Cl)cc2)=NN1)/C(=C/c1cccc([N+](=O)[O-])c1)n1cncn1] | −7.4 |
| 9 | 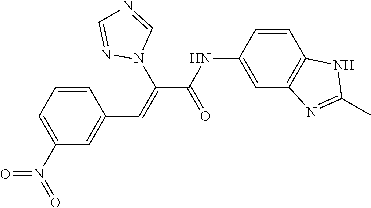 [SMILES: Cc1nc2cc(NC(=O)/C(=C/c3cccc([N+](=O)[O-])c3)n3cncn3)ccc2[nH]1] | −7.4 |
| 10 | 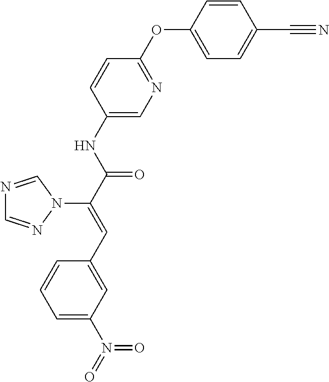 [SMILES: N#Cc1ccc(Oc2ccc(NC(=O)/C(=C/c3cccc([N+](=O)[O-])c3)n3cncn3)cn2)cc1] | −7.3 |
| 11 | 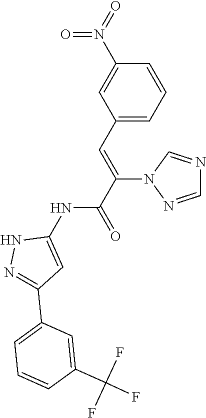 [SMILES: O=C(Nc1cc(-c2cccc(C(F)(F)F)c2)n[nH]1)/C(=C/c1cccc([N+](=O)[O-])c1)n1cncn1] | −7.3 |
| 12 | 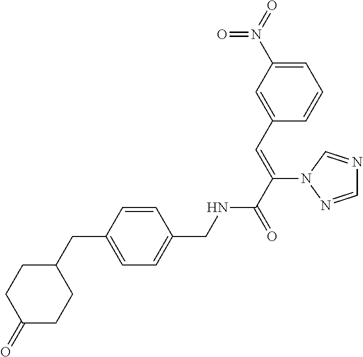 [SMILES: O=C1CCC(Cc2ccc(CNC(=O)/C(=C/c3cccc([N+](=O)[O-])c3)n3cncn3)cc2)CC1] | −7.3 |
| 13 | 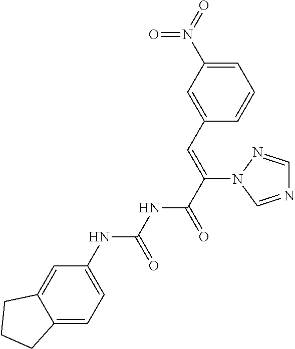 [SMILES: O=C(NC(=O)/C(=C/c1cccc([N+](=O)[O-])c1)n1cncn1)Nc1ccc2c(c1)CCC2] | −7.3 |
| 14 | 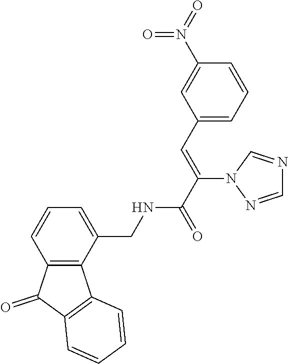 [SMILES: O=C(NCc1cccc2c1-c1ccccc1C2=O)/C(=C/c1cccc([N+](=O)[O-])c1)n1cncn1] | −7.3 |
| 15 | 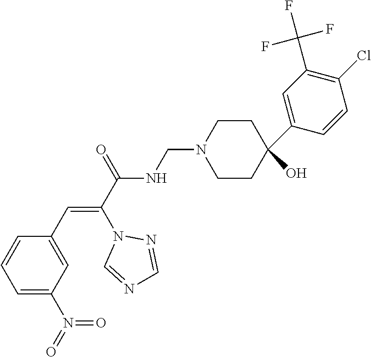 | −7.3 |
| 16 | 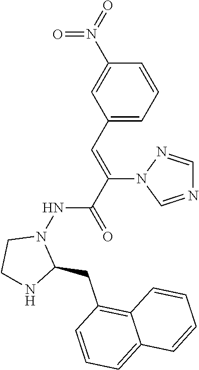 [SMILES: O=C(NN1CCN[C@@H]1Cc1cccc2ccccc12)/C(=C/c1cccc([N+](=O)[O-])c1)n1cncn1] | −7.3 |
| 17 | 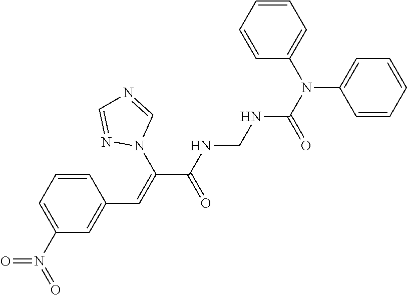 [SMILES: O=C(NCNC(=O)N(c1ccccc1)c1ccccc1)/C(=C/c1cccc([N+](=O)[O-])c1)n1cncn1] | −7.3 |
| 18 | 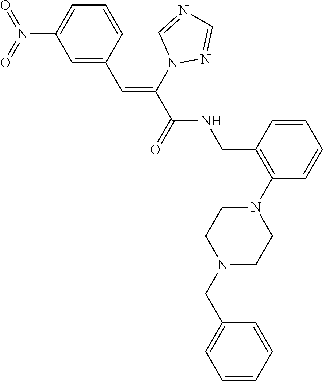 [SMILES: O=C(NCc1ccccc1N1CCN(Cc2ccccc2)CC1)/C(=C/c1cccc([N+](=O)[O-])c1)n1cncn1] | −7.3 |
| 19 | 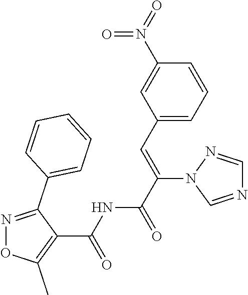 [SMILES: Cc1onc(-c2ccccc2)c1C(=O)NC(=O)/C(=C/c1cccc([N+](=O)[O-])c1)n1cncn1] | −7.3 |
| 20 | 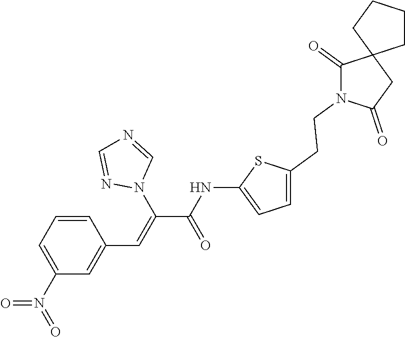 [SMILES: O=C(Nc1ccc(CCN2C(=O)CC3(CCCC3)C2=O)s1)/C(=C/c1cccc([N+](=O)[O-])c1)n1cncn1] | −7.2 |
| 21 | 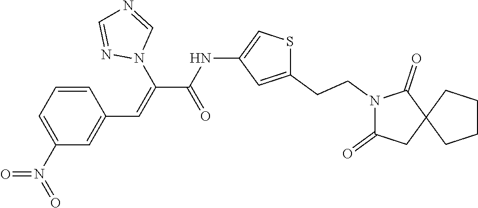 [SMILES: O=C(Nc1csc(CCN2C(=O)CC3(CCCC3)C2=O)c1)/C(=C/c1cccc([N+](=O)[O-])c1)n1cncn1] | −7.2 |
| 22 | 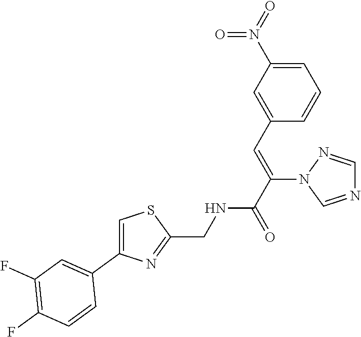 [SMILES: O=C(NCc1nc(-c2ccc(F)c(F)c2)cs1)/C(=C/c1cccc([N+](=O)[O-])c1)n1cncn1] | −7.2 |
| 23 | 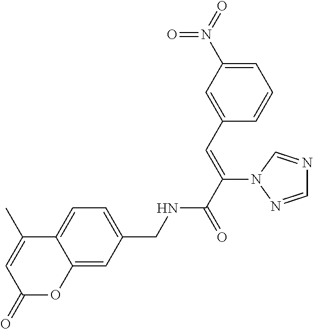 [SMILES: Cc1cc(=O)oc2cc(CNC(=O)/C(=C/c3cccc([N+](=O)[O-])c3)n3cncn3)ccc12] | −7.2 |
| 24 | 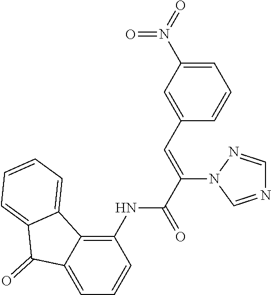 [SMILES: O=C(Nc1cccc2c1-c1ccccc1C2=O)/C(=C/c1cccc([N+](=O)[O-])c1)n1cncn1] | −7.2 |
| 25 | 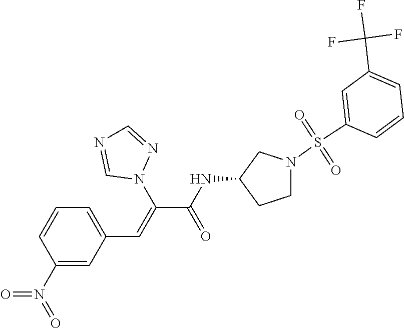 [SMILES: O=C(N[C@H]1CCN(S(=O)(=O)c2cccc(C(F)(F)F)c2)C1)/C(=C/c1cccc([N+](=O)[O-])c1)n1cncn1] | −7.2 |
| 26 | 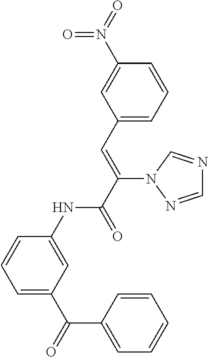 [SMILES: O=C(Nc1cccc(C(=O)c2ccccc2)c1)/C(=C/c1cccc([N+](=O)[O-])c1)n1cncn1] | −7.2 |
| 27 | 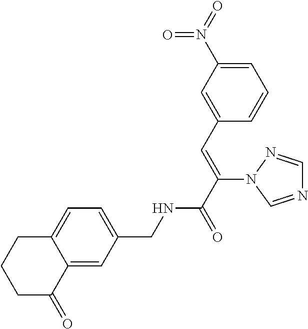 [SMILES: O=C(NCc1ccc2c(c1)C(=O)CCC2)/C(=C/c1cccc([N+](=O)[O-])c1)n1cncn1] | −7.2 |
| 28 | 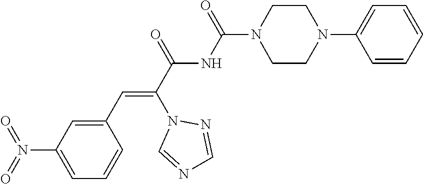 [SMILES: O=C(NC(=O)N1CCN(c2ccccc2)CC1)/C(=C/c1cccc([N+](=O)[O-])c1)n1cncn1] | −7.2 |
| 29 | 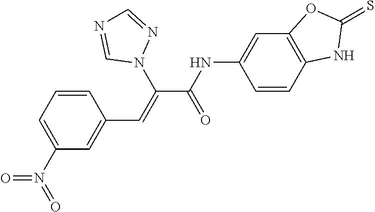 [SMILES: O=C(Nc1ccc2[nH]c(=S)oc2c1)/C(=C/c1cccc([N+](=O)[O-])c1)n1cncn1] | −7.2 |
| 30 | 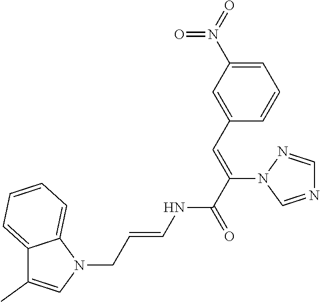 [SMILES: Cc1cn(C/C=C/NC(=O)/C(=C/c2cccc([N+](=O)[O-])c2)n2cncn2)c2ccccc12] | −7.2 |
| 31 | 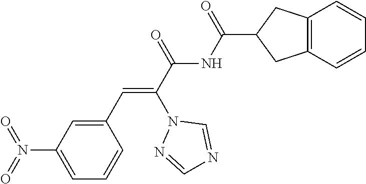 [SMILES: O=C(NC(=O)C1Cc2ccccc2C1)/C(=C/c1cccc([N+](=O)[O-])c1)n1cncn1] | −7.2 |
| 32 | 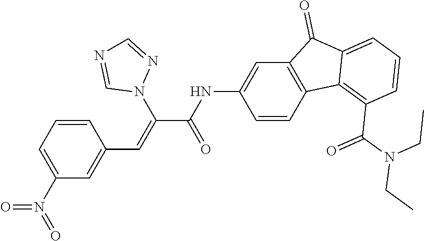 [SMILES: CCN(CC)C(=O)c1cccc2c1-c1ccc(NC(=O)/C(=C/c3cccc([N+](=O)[O-])c3)n3cncn3)cc1C2=O] | −7.2 |
| 33 | 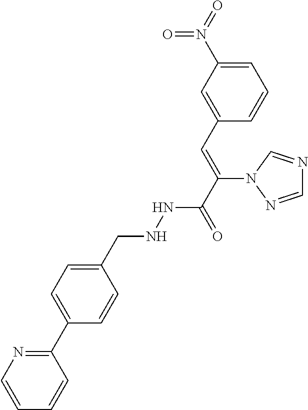 [SMILES: O=C(NNCc1ccc(-c2ccccn2)cc1)/C(=C/c1cccc([N+](=O)[O-])c1)n1cncn1] | −7.2 |
| 34 | 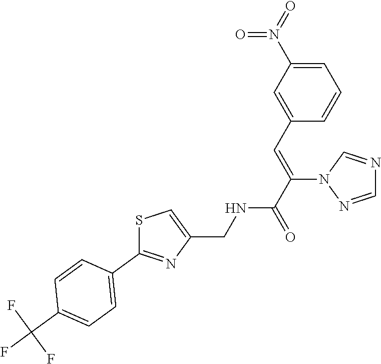 [SMILES: O=C(NCc1csc(-c2ccc(C(F)(F)F)cc2)n1)/C(=C/c1cccc([N+](=O)[O-])c1)n1cncn1] | −7.2 |
| 35 | 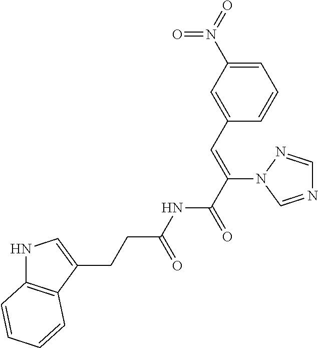 [SMILES: O=C(CCc1c[nH]c2ccccc12)NC(=O)/C(=C/c1cccc([N+](=O)[O-])c1)n1cncn1] | −7.2 |
| 36 | 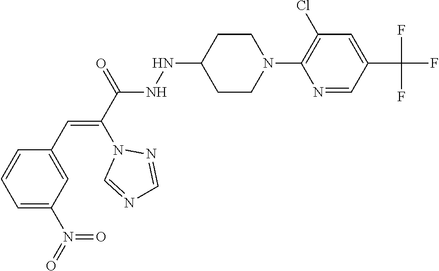 [SMILES: O=C(NNC1CCN(c2ncc(C(F)(F)F)cc2Cl)CC1)/C(=C/c1cccc([N+](=O)[O-])c1)n1cncn1] | −7.2 |
| 37 | 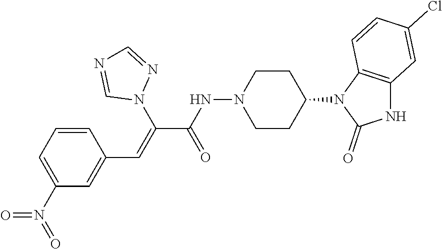 | −7.2 |
| 38 | 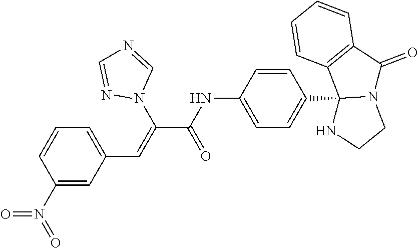 [SMILES: O=C(Nc1ccc([C@@]23NCCN2C(=O)c2ccccc23)cc1)/C(=C/c1cccc([N+](=O)[O-])c1)n1cncn1] | −7.2 |
| 39 | 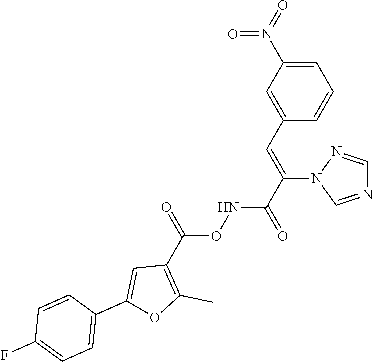 [SMILES: Cc1oc(-c2ccc(F)cc2)cc1C(=O)ONC(=O)/C(=C/c1cccc([N+](=O)[O-])c1)n1cncn1] | −7.2 |
| 40 | 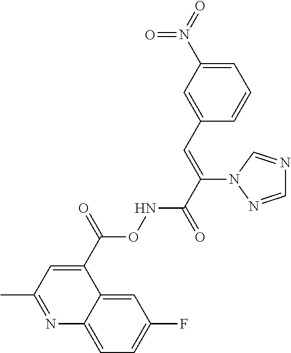 [SMILES: Cc1cc(C(=O)ONC(=O)/C(=C/c2cccc([N+](=O)[O-])c2)n2cncn2)c2cc(F)ccc2n1] | −7.2 |
| 41 | 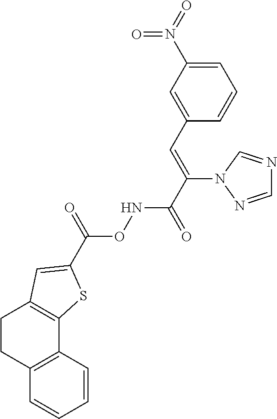 [SMILES: O=C(NOC(=O)c1cc2c(s1)-c1ccccc1CC2)/C(=C/c1cccc([N+](=O)[O-])c1)n1cncn1] | −7.2 |
| 42 | 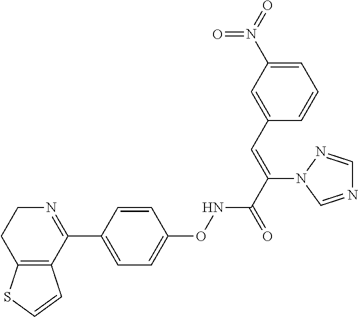 [SMILES: O=C(NOc1ccc(C2=NCCc3sccc32)cc1)/C(=C/c1cccc([N+](=O)[O-])c1)n1cncn1] | −7.2 |
| 43 | 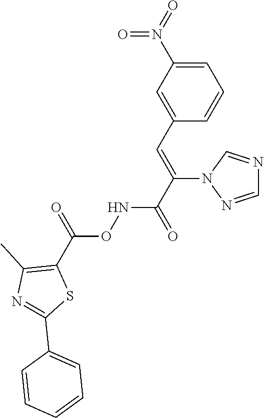 [SMILES: Cc1nc(-c2ccccc2)sc1C(=O)ONC(=O)/C(=C/c1cccc([N+](=O)[O-])c1)n1cncn1] | −7.2 |
| 44 | 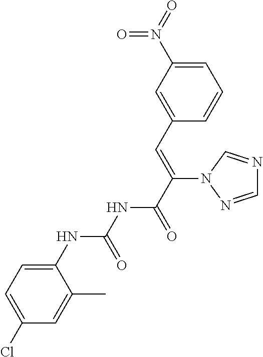 [SMILES: Cc1cc(Cl)ccc1NC(=O)NC(=O)/C(=C/c1cccc([N+](=O)[O-])c1)n1cncn1] | −7.2 |
| 45 | 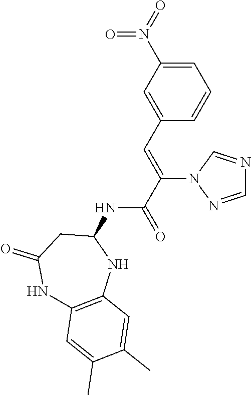 [SMILES: Cc1cc2c(cc1C)N[C@H](NC(=O)/C(=C/c1cccc([N+](=O)[O-])c1)n1cncn1)CC(=O)N2] | −7.1 |
| 46 | 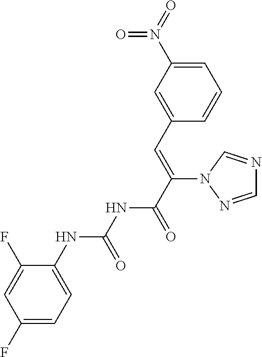 [SMILES: O=C(NC(=O)/C(=C/c1cccc([N+](=O)[O-])c1)n1cncn1)Nc1ccc(F)cc1F] | −7.1 |
| 47 | 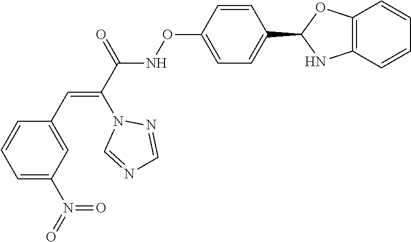 [SMILES: O=C(NOc1ccc([C@@H]2Nc3ccccc3O2)cc1)/C(=C/c1cccc([N+](=O)[O-])c1)n1cncn1] | −7.1 |
| 48 | 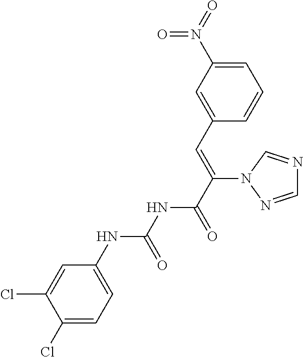 [SMILES: O=C(NC(=O)/C(=C/c1cccc([N+](=O)[O-])c1)n1cncn1)Nc1ccc(Cl)c(Cl)c1] | −7.1 |
| 49 | 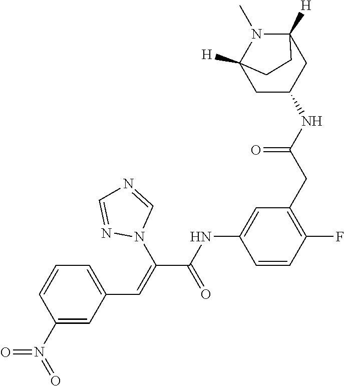 [SMILES: CN1[C@@H]2CC[C@H]1C[C@H](NC(=O)Cc1cc(NC(=O)/C(=C/c3cccc([N+](=O)[O-])c3)n3cncn3)ccc1F)C2] | −7.1 |
| 50 | 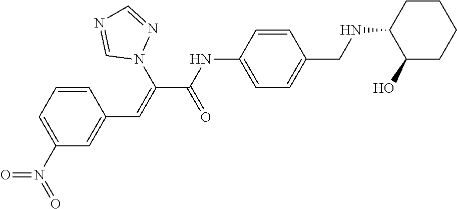 [SMILES: O=C(Nc1ccc(CN[C@@H]2CCCC[C@H]2O)cc1)/C(=C/c1cccc([N+](=O)[O-])c1)n1cncn1] | −7.1 |
| 51 | 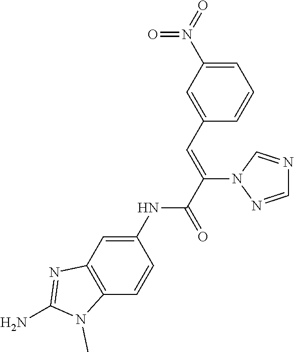 [SMILES: Cn1c(N)nc2cc(NC(=O)/C(=C/c3cccc([N+](=O)[O-])c3)n3cncn3)ccc21] | −7.1 |
| 52 | 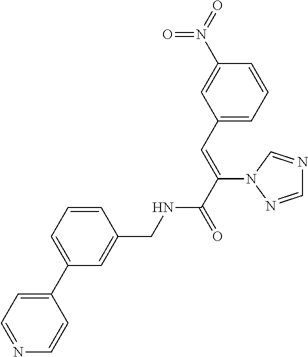 [SMILES: O=C(NCc1cccc(-c2ccncc2)c1)/C(=C/c1cccc([N+](=O)[O-])c1)n1cncn1] | −7.1 |
| 53 | 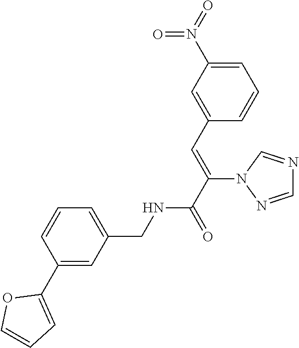 [SMILES: O=C(NCc1cccc(-c2ccco2)c1)/C(=C/c1cccc([N+](=O)[O-])c1)n1cncn1] | −7.1 |
| 54 | 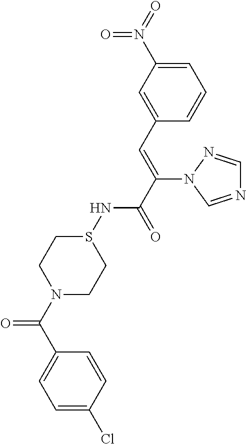 | −7.1 |
| 55 | 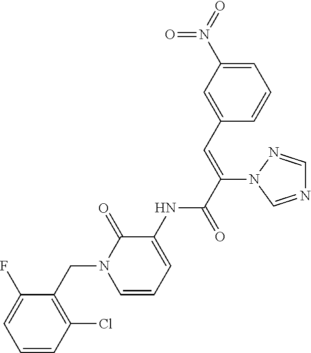 [SMILES: O=C(Nc1cccn(Cc2c(F)cccc2Cl)c1=O)/C(=C/c1cccc([N+](=O)[O-])c1)n1cncn1] | −7.1 |
| 56 | 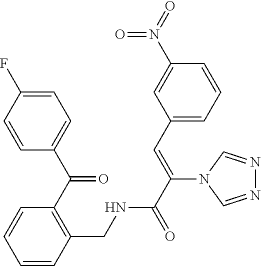 [SMILES: O=C(NCc1ccccc1C(=O)c1ccc(F)cc1)/C(=C/c1cccc([N+](=O)[O-])c1)n1cnnc1] | −7.1 |
| 57 | 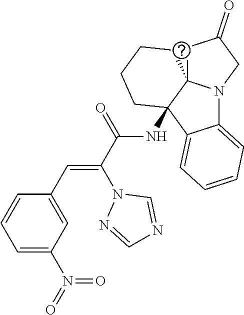 [SMILES: O=C1CN2c3ccccc3[C@]3(NC(=O)/C(=C/c4cccc([N+](=O)[O-])c4)n4cncn4)CCCC[C@]23C1] | −7.1 |
| 58 | 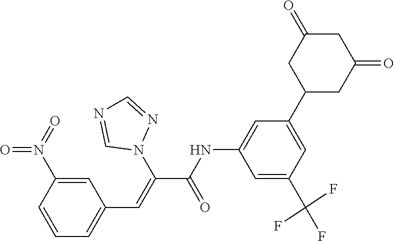 [SMILES: O=C1CC(=O)CC(c2cc(NC(=O)/C(=C/c3cccc([N+](=O)[O-])c3)n3cncn3)cc(C(F)(F)F)c2)C1] | −7.1 |
| 59 | 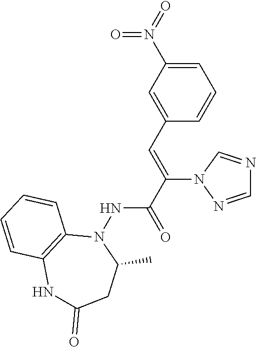 [SMILES: C[C@@H]1CC(=O)Nc2ccccc2N1NC(=O)/C(=C/c1cccc([N+](=O)[O-])c1)n1cncn1] | −7.1 |
| 60 | 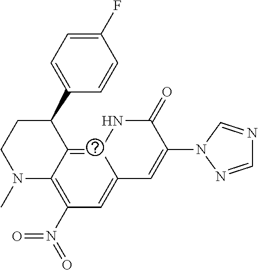 [SMILES: CN1CC[C@@H](c2ccc(F)cc2)c2c1c([N+](=O)[O-])cc1cc(-n3cncn3)c(=O)[nH]c21] | −7.1 |
| 61 | 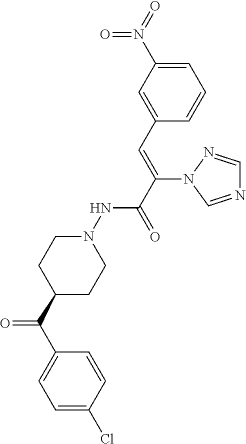 | −7.1 |
| 62 | 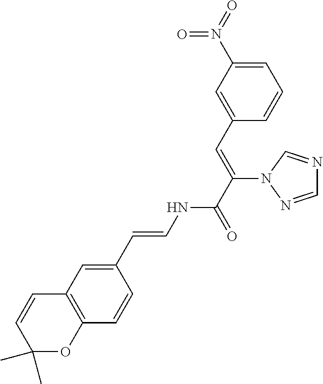 [SMILES: CC1(C)C=Cc2cc(/C=C/NC(=O)/C(=C/c3cccc([N+](=O)[O-])c3)n3cncn3)ccc2O1] | −7.1 |
| 63 | 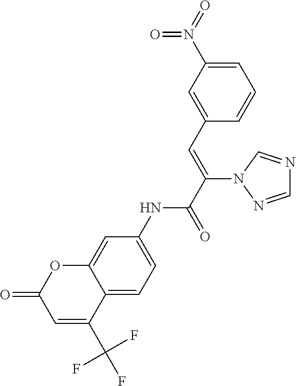 [SMILES: O=C(Nc1ccc2c(C(F)(F)F)cc(=O)oc2c1)/C(=C/c1cccc([N+](=O)[O-])c1)n1cncn1] | −7.1 |
| 64 | 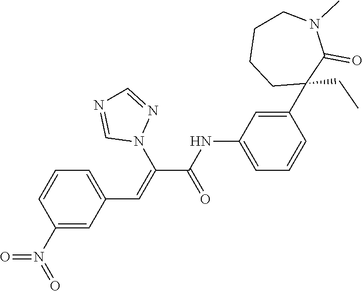 [SMILES: CC[C@]1(c2cccc(NC(=O)/C(=C/c3cccc([N+](=O)[O-])c3)n3cncn3)c2)CCCCN(C)C1=O] | −7.1 |
| 65 | 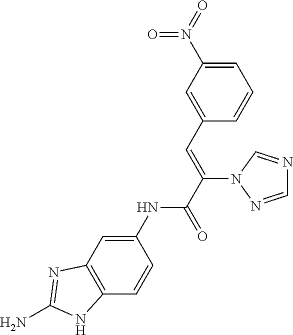 [SMILES: Nc1nc2cc(NC(=O)/C(=C/c3cccc([N+](=O)[O-])c3)n3cncn3)ccc2[nH]1] | −7.1 |
| 66 | 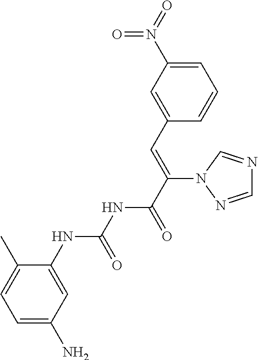 [SMILES: Cc1ccc(N)cc1NC(=O)NC(=O)/C(=C/c1cccc([N+](=O)[O-])c1)n1cncn1] | −7.1 |
| 67 | 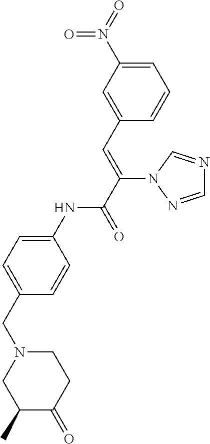 [SMILES: C[C@H]1CN(Cc2ccc(NC(=O)/C(=C/c3cccc([N+](=O)[O-])c3)n3cncn3)cc2)CCC1=O] | −7.1 |
| 68 | 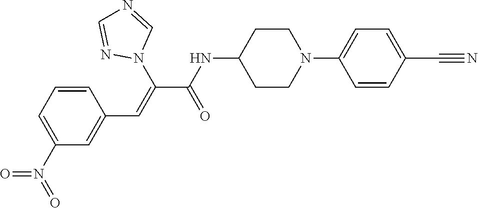 [SMILES: N#Cc1ccc(N2CCC(NC(=O)/C(=C/c3cccc([N+](=O)[O-])c3)n3cncn3)CC2)cc1] | −7.1 |
| 69 | 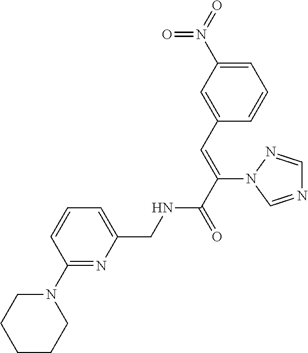 [SMILES: O=C(NCc1cccc(N2CCCCC2)n1)/C(=C/c1cccc([N+](=O)[O-])c1)n1cncn1] | −7.1 |
| 70 | 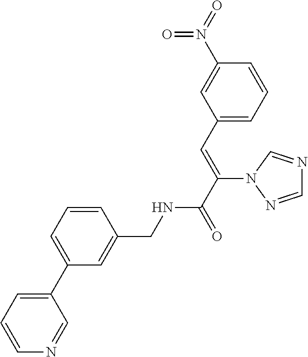 [SMILES: O=C(NCc1cccc(-c2cccnc2)c1)/C(=C/c1cccc([N+](=O)[O-])c1)n1cncn1] | −7.1 |
| 71 | 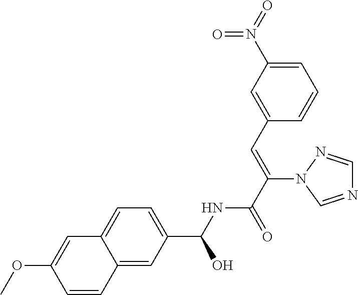 [SMILES: COc1ccc2cc([C@H](O)NC(=O)/C(=C/c3cccc([N+](=O)[O-])c3)n3cncn3)ccc2c1] | −7.1 |
| 72 | 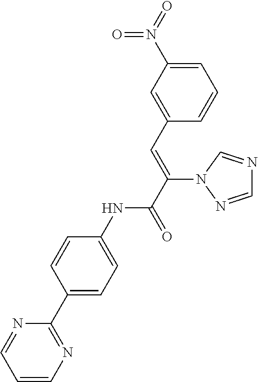 [SMILES: O=C(Nc1ccc(-c2ncccn2)cc1)/C(=C/c1cccc([N+](=O)[O-])c1)n1cncn1] | −7.1 |
| 73 | 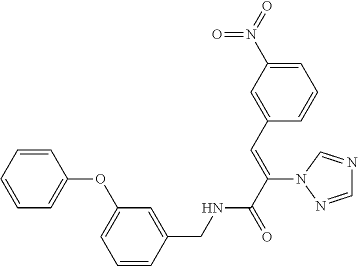 [SMILES: O=C(NCc1cccc(Oc2ccccc2)c1)/C(=C/c1cccc([N+](=O)[O-])c1)n1cncn1] | −7.1 |
| 74 | 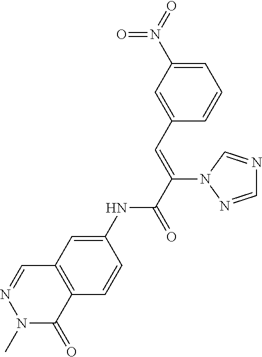 [SMILES: Cn1ncc2cc(NC(=O)/C(=C/c3cccc([N+](=O)[O-])c3)n3cncn3)ccc2c1=O] | −7.1 |
| 75 | 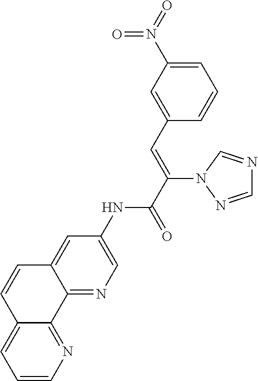 [SMILES: O=C(Nc1cnc2c(ccc3cccnc32)c1)/C(=C/c1cccc([N+](=O)[O-])c1)n1cncn1] | −7.1 |
| 76 | 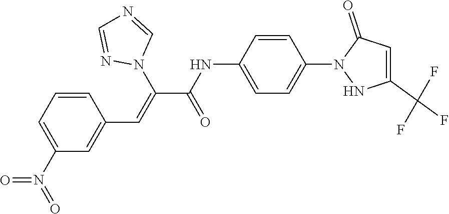 [SMILES: O=C(Nc1ccc(-n2[nH]c(C(F)(F)F)cc2=O)cc1)/C(=C/c1cccc([N+](=O)[O-])c1)n1cncn1] | −7.1 |
| 77 | 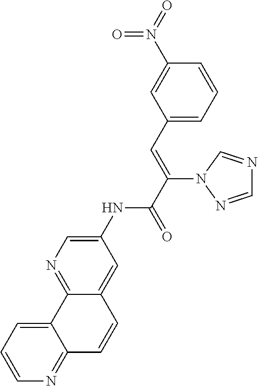 [SMILES: O=C(Nc1cnc2c(ccc3ncccc32)c1)/C(=C/c1cccc([N+](=O)[O-])c1)n1cncn1] | −7.1 |
| 78 | 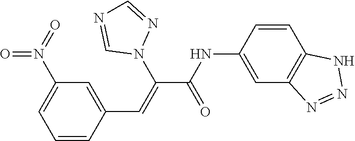 [SMILES: O=C(Nc1ccc2[nH]nnc2c1)/C(=C/c1cccc([N+](=O)[O-])c1)n1cncn1] | −7.0 |
| 79 | 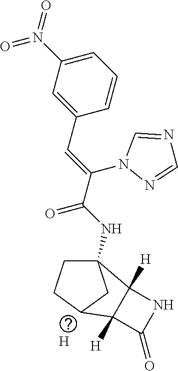 | −7.0 |
| 80 | 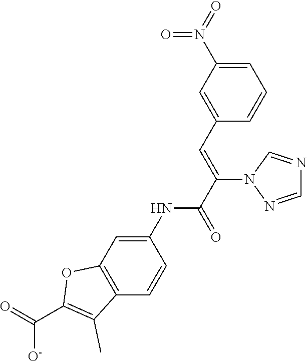 [SMILES: Cc1c(C(=O)[O-])oc2cc(NC(=O)/C(=C/c3cccc([N+](=O)[O-])c3)n3cncn3)ccc12] | −7.0 |
| 81 | 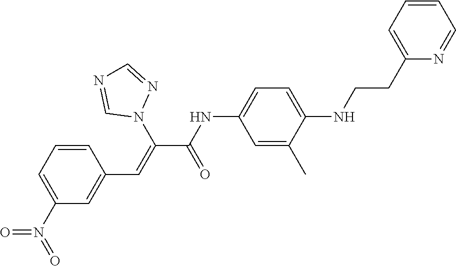 [SMILES: Cc1cc(NC(=O)/C(=C/c2cccc([N+](=O)[O-])c2)n2cncn2)ccc1NCCc1ccccn1] | −7.0 |
| 82 | 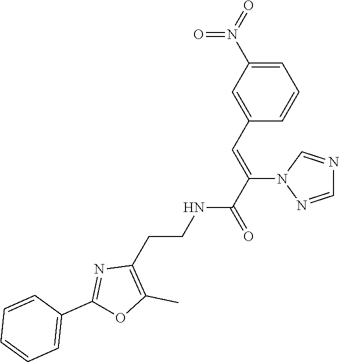 [SMILES: Cc1oc(-c2ccccc2)nc1CCNC(=O)/C(=C/c1cccc([N+](=O)[O-])c1)n1cncn1] | −7.0 |
| 83 | 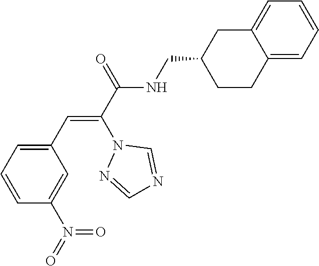 [SMILES: O=C(NC[C@H]1CCc2ccccc2C1)/C(=C/c1cccc([N+](=O)[O-])c1)n1cncn1] | −7.0 |
| 84 | 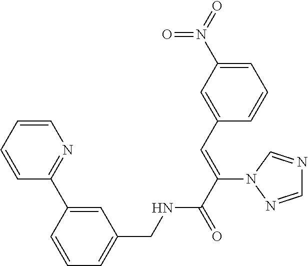 [SMILES: O=C(NCc1cccc(-c2ccccn2)c1)/C(=C/c1cccc([N+](=O)[O-])c1)n1cncn1] | −7.0 |
| 85 | 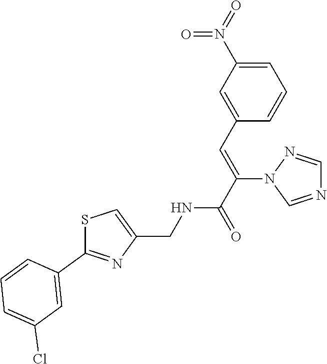 [SMILES: O=C(NCc1csc(-c2cccc(Cl)c2)n1)/C(=C/c1cccc([N+](=O)[O-])c1)n1cncn1] | −7.0 |
| 86 | 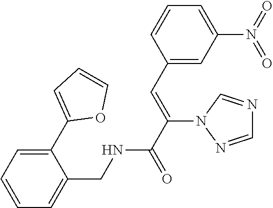 [SMILES: O=C(NCc1ccccc1-c1ccco1)/C(=C/c1cccc([N+](=O)[O-])c1)n1cncn1] | −7.0 |
| 87 | 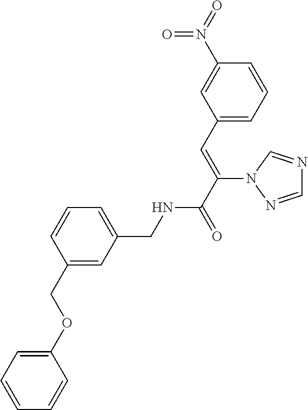 [SMILES: O=C(NCc1cccc(COc2ccccc2)c1)/C(=C/c1cccc([N+](=O)[O-])c1)n1cncn1] | −7.0 |
| 88 | 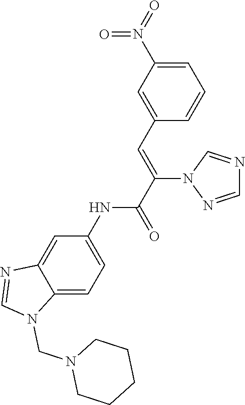 [SMILES: O=C(Nc1ccc2c(c1)ncn2CN1CCCCC1)/C(=C/c1cccc([N+](=O)[O-])c1)n1cncn1] | −7.0 |
| 89 | 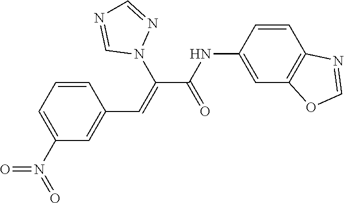 [SMILES: O=C(Nc1ccc2ncoc2c1)/C(=C/c1cccc([N+](=O)[O-])c1)n1cncn1] | −7.0 |
| 90 | 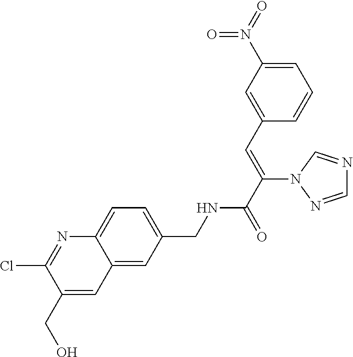 [SMILES: O=C(NCc1ccc2nc(Cl)c(CO)cc2c1)/C(=C/c1cccc([N+](=O)[O-])c1)n1cncn1] | −7.0 |
| 91 | 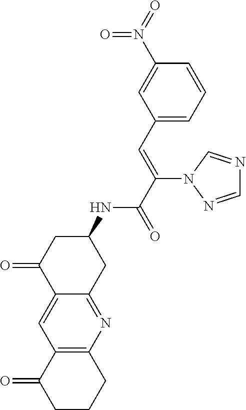 [SMILES: O=C(N[C@@H]1CC(=O)c2cc3c(nc2C1)CCCC3=O)/C(=C/c1cccc([N+](=O)[O-])c1)n1cncn1] | −7.0 |
| 92 | 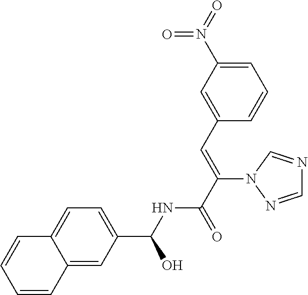 [SMILES: O=C(N[C@@H](O)c1ccc2ccccc2c1)/C(=C/c1cccc([N+](=O)[O-])c1)n1cncn1] | −7.0 |
| 93 | 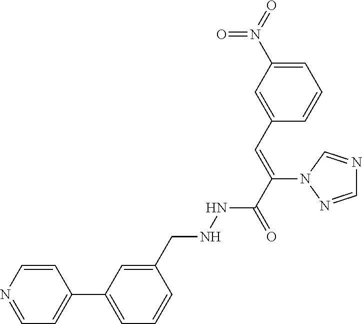 [SMILES: O=C(NNCc1cccc(-c2ccncc2)c1)/C(=C/c1cccc([N+](=O)[O-])c1)n1cncn1] | −7.0 |
| 94 | 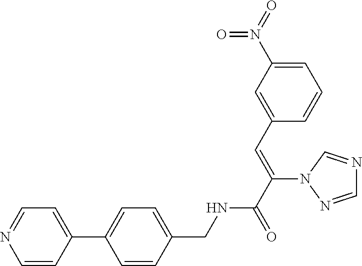 [SMILES: O=C(NCc1ccc(-c2ccncc2)cc1)/C(=C/c1cccc([N+](=O)[O-])c1)n1cncn1] | −7.0 |
| 95 | 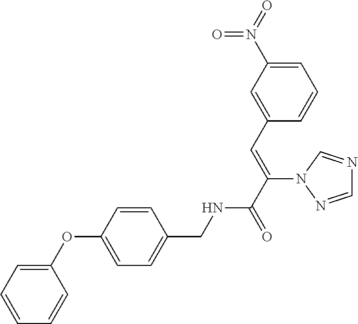 [SMILES: O=C(NCc1ccc(Oc2ccccc2)cc1)/C(=C/c1cccc([N+](=O)[O-])c1)n1cncn1] | −7.0 |
| 96 | 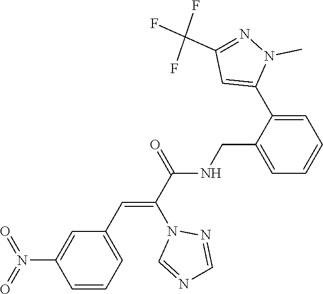 [SMILES: Cn1nc(C(F)(F)F)cc1-c1ccccc1CNC(=O)/C(=C/c1cccc([N+](=O)[O-])c1)n1cncn1] | −7.0 |
| 97 | 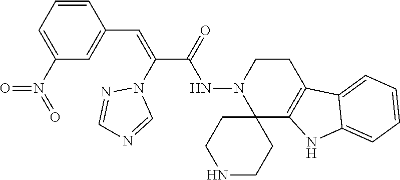 [SMILES: O=C(NN1CCc2c([nH]c3ccccc23)C12CCNCC2)/C(=C/c1cccc([N+](=O)[O-])c1)n1cncn1] | −7.0 |
| 98 | 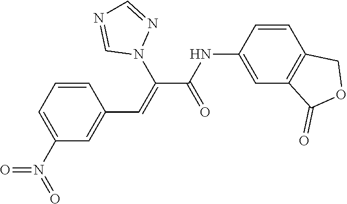 [SMILES: O=C(Nc1ccc2c(c1)C(=O)OC2)/C(=C/c1cccc([N+](=O)[O-])c1)n1cncn1] | −7.0 |
| 99 | 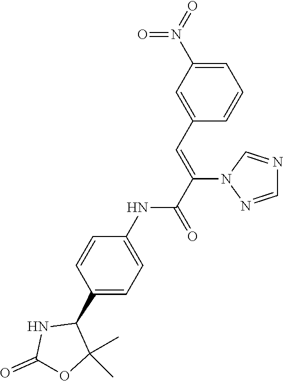 [SMILES: CC1(C)OC(=O)N[C@H]1c1ccc(NC(=O)/C(=C/c2cccc([N+](=O)[O-])c2)n2cncn2)cc1] | −7.0 |
| 100 | 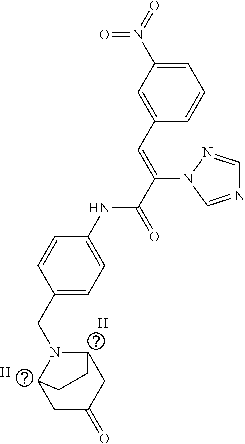 | −7.0 |
| 101 | 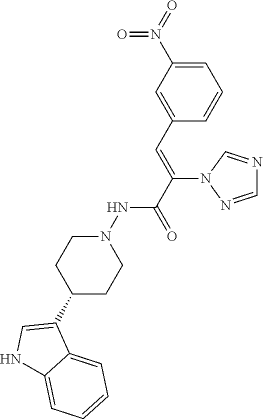 | −7.0 |
| 102 | 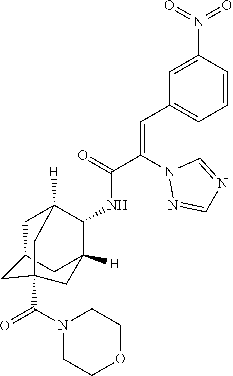 [SMILES: O=C(N[C@H]1[C@@H]2C[C@@H]3C[C@H]1C[C@@](C(=O)N1CCOCC1)(C3)C2)/C(=C/c1cccc([N+](=O)[O-])c1)n1cncn1] | −7.0 |
| 103 | 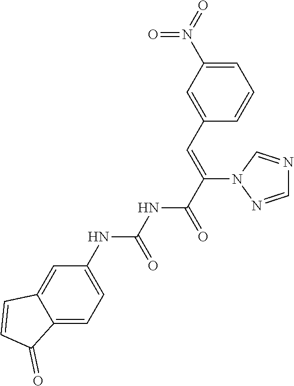 [SMILES: O=C(NC(=O)/C(=C/c1cccc([N+](=O)[O-])c1)n1cncn1)Nc1ccc2c(c1)C=CC2=O] | −7.0 |
| 104 | 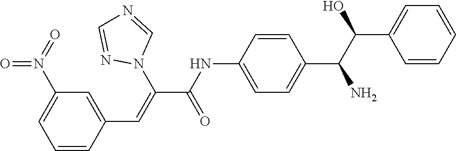 [SMILES: N[C@@H](c1ccc(NC(=O)/C(=C/c2cccc([N+](=O)[O-])c2)n2cncn2)cc1)[C@@H](O)c1ccccc1] | −7.0 |
| 105 | 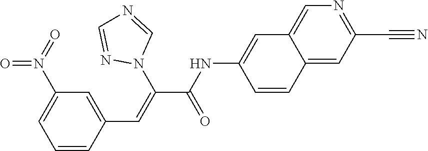 [SMILES: N#Cc1cc2ccc(NC(=O)/C(=C/c3cccc([N+](=O)[O-])c3)n3cncn3)cc2cn1] | −7.0 |
| 106 | 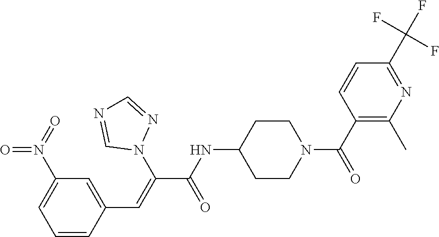 [SMILES: Cc1nc(C(F)(F)F)ccc1C(=O)N1CCC(NC(=O)/C(=C/c2cccc([N+](=O)[O-])c2)n2cncn2)CC1] | −7.0 |
| 107 | 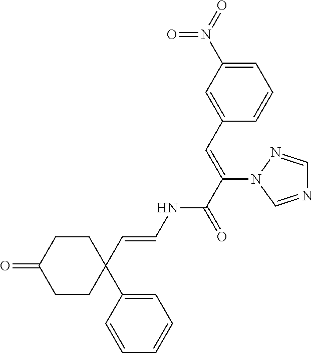 [SMILES: O=C1CCC(/C=C/NC(=O)/C(=C/c2cccc([N+](=O)[O-])c2)n2cncn2)(c2ccccc2)CC1] | −7.0 |
| 108 | 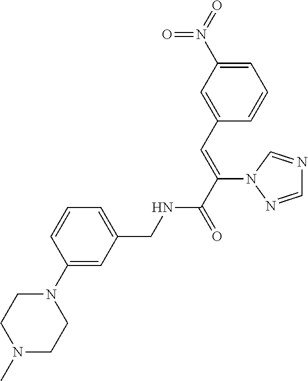 [SMILES: CN1CCN(c2cccc(CNC(=O)/C(=C/c3cccc([N+](=O)[O-])c3)n3cncn3)c2)CC1] | −7.0 |
| 109 | 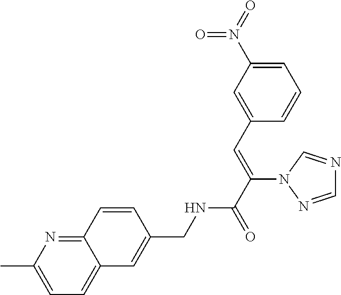 [SMILES: Cc1ccc2cc(CNC(=O)/C(=C/c3cccc([N+](=O)[O-])c3)n3cncn3)ccc2n1] | −7.0 |
| 110 | 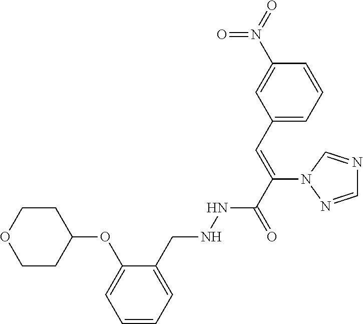 [SMILES: O=C(NNCc1ccccc1OC1CCOCC1)/C(=C/c1cccc([N+](=O)[O-])c1)n1cncn1] | −7.0 |
| 111 | 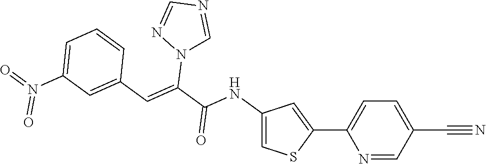 [SMILES: N#Cc1ccc(-c2cc(NC(=O)/C(=C/c3cccc([N+](=O)[O-])c3)n3cncn3)cs2)nc1] | −7.0 |
| 112 | 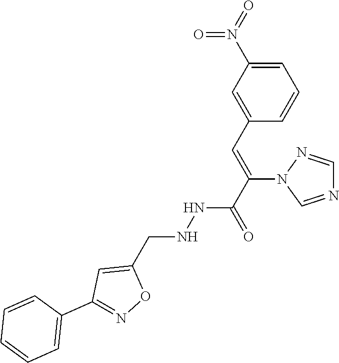 [SMILES: O=C(NNCc1cc(-c2ccccc2)no1)/C(=C/c1cccc([N+](=O)[O-])c1)n1cncn1] | −7.0 |
| 113 | 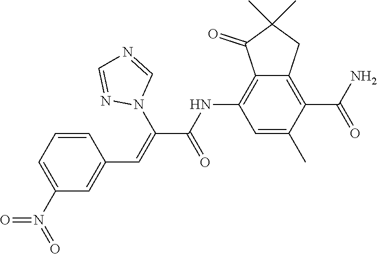 [SMILES: Cc1cc(NC(=O)/C(=C/c2cccc([N+](=O)[O-])c2)n2cncn2)c2c(c1C(N)=O)CC(C)(C)C2=O] | −7.0 |
| 114 | 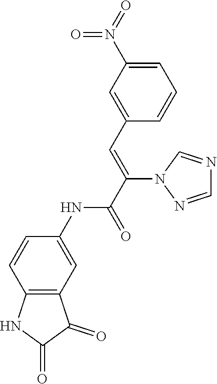 [SMILES: O=C1Nc2ccc(NC(=O)/C(=C/c3cccc([N+](=O)[O-])c3)n3cncn3)cc2C1=O] | −7.0 |
| 115 | 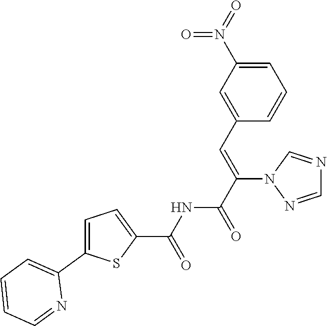 [SMILES: O=C(NC(=O)c1ccc(-c2ccccn2)s1)/C(=C/c1cccc([N+](=O)[O-])c1)n1cncn1] | −7.0 |
| 116 | 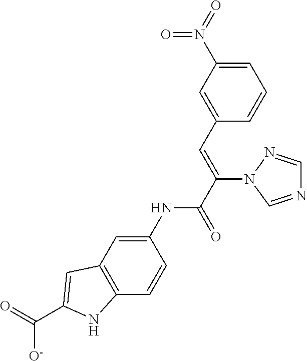 [SMILES: O=C(Nc1ccc2[nH]c(C(=O)[O-])cc2c1)/C(=C/c1cccc([N+](=O)[O-])c1)n1cncn1] | −7.0 |
| 117 | 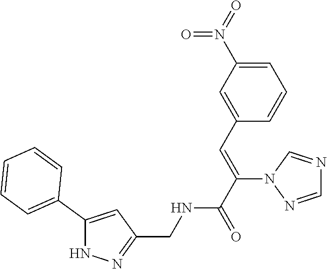 [SMILES: O=C(NCc1cc(-c2ccccc2)[nH]n1)/C(=C/c1cccc([N+](=O)[O-])c1)n1cncn1] | −7.0 |
| 118 | 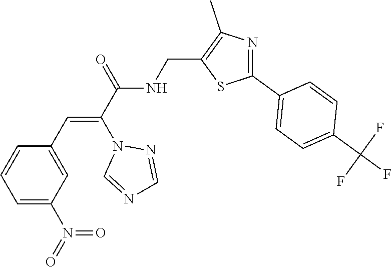 [SMILES: Cc1nc(-c2ccc(C(F)(F)F)cc2)sc1CNC(=O)/C(=C/c1cccc([N+](=O)[O-])c1)n1cncn1] | −7.0 |
| 119 | 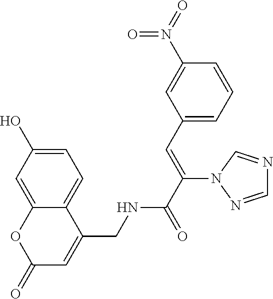 [SMILES: O=C(NCc1cc(=O)oc2cc(O)ccc12)/C(=C/c1cccc([N+](=O)[O-])c1)n1cncn1] | −7.0 |
| 120 | 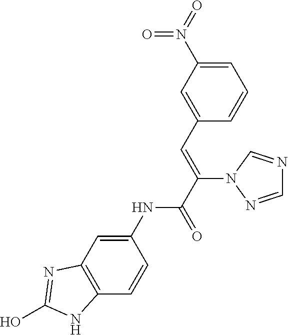 [SMILES: O=C(Nc1ccc2[nH]c(O)nc2c1)/C(=C/c1cccc([N+](=O)[O-])c1)n1cncn1] | −7.0 |
| 121 | 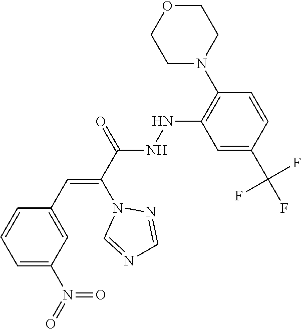 [SMILES: O=C(NNc1cc(C(F)(F)F)ccc1N1CCOCC1)/C(=C/c1cccc([N+](=O)[O-])c1)n1cncn1] | −7.0 |
| 122 | 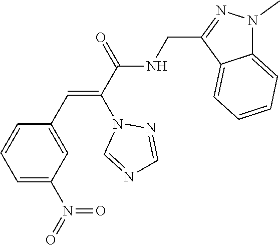 [SMILES: Cn1nc(CNC(=O)/C(=C/c2cccc([N+](=O)[O-])c2)n2cncn2)c2ccccc21] | −7.0 |
| 123 | 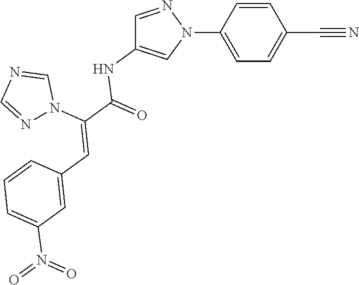 [SMILES: N#Cc1ccc(-n2cc(NC(=O)/C(=C/c3cccc([N+](=O)[O-])c3)n3cncn3)cn2)cc1] | −7.0 |
| 124 | 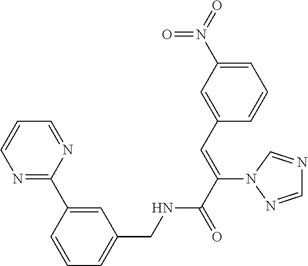 [SMILES: O=C(NCc1cccc(-c2ncccn2)c1)/C(=C/c1cccc([N+](=O)[O-])c1)n1cncn1] | −7.0 |
| 125 | 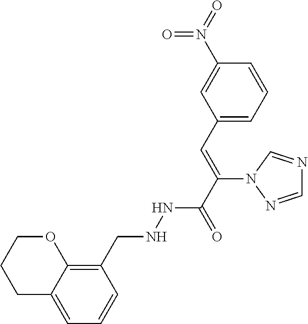 [SMILES: O=C(NNCc1cccc2c1OCCC2)/C(=C/c1cccc([N+](=O)[O-])c1)n1cncn1] | −7.0 |
| 126 | 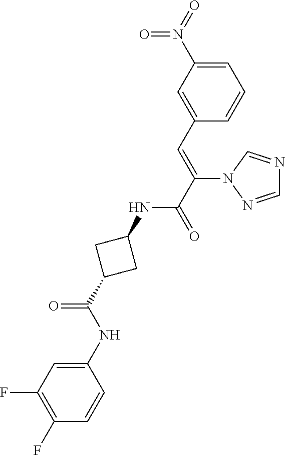 [SMILES: O=C(N[C@H]1C[C@H](C(=O)Nc2ccc(F)c(F)c2)C1)/C(=C/c1cccc([N+](=O)[O-])c1)n1cncn1] | −7.0 |
| 127 | 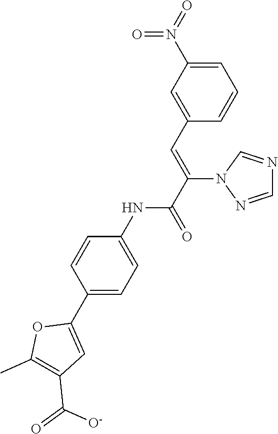 [SMILES: Cc1oc(-c2ccc(NC(=O)/C(=C/c3cccc([N+](=O)[O-])c3)n3cncn3)cc2)cc1C(=O)[O-]] | −7.0 |
| 128 | 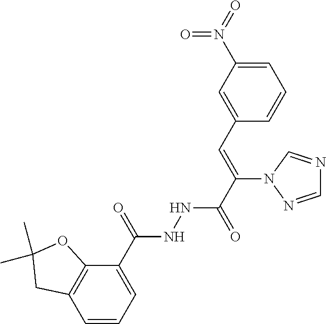 [SMILES: CC1(C)Cc2cccc(C(=O)NNC(=O)/C(=C/c3cccc([N+](=O)[O-])c3)n3cncn3)c2O1] | −7.0 |
| 129 | 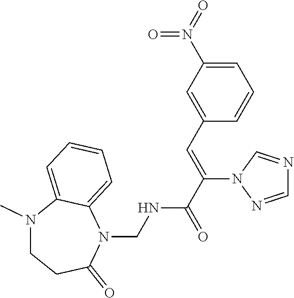 [SMILES: CN1CCC(=O)N(CNC(=O)/C(=C/c2cccc([N+](=O)[O-])c2)n2cncn2)c2ccccc21] | −7.0 |
| 130 | 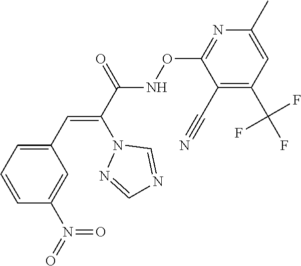 [SMILES: Cc1cc(C(F)(F)F)c(C#N)c(ONC(=O)/C(=C/c2cccc([N+](=O)[O-])c2)n2cncn2)n1] | −7.0 |
| 131 | 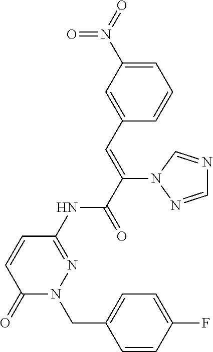 [SMILES: O=C(Nc1ccc(=O)n(Cc2ccc(F)cc2)n1)/C(=C/c1cccc([N+](=O)[O-])c1)n1cncn1] | −7.0 |
| 132 | 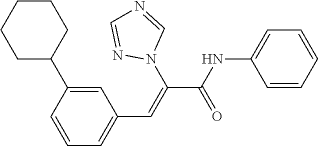 [SMILES: O=C(Nc1ccccc1)/C(=C/c1cccc(C2CCCCC2)c1)n1cncn1] | −7.2 |
| 133 | 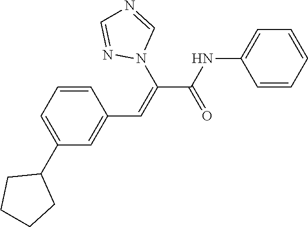 [SMILES: O=C(Nc1ccccc1)/C(=C/c1cccc(C2CCCC2)c1)n1cncn1] | −7.1 |
| 134 | 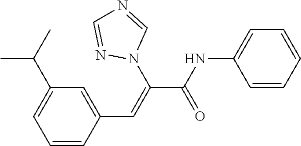 [SMILES: CC(C)c1cccc(/C=C(/C(=O)Nc2ccccc2)n2cncn2)c1] | −7.0 |
| 135 | 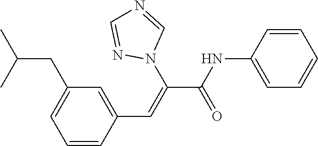 [SMILES: CC(C)Cc1cccc(/C=C(/C(=O)Nc2ccccc2)n2cncn2)c1] | −6.8 |
| 136 | 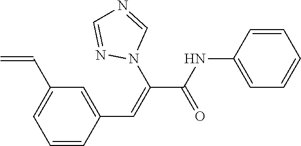 [SMILES: C=Cc1cccc(/C=C(/C(=O)Nc2ccccc2)n2cncn2)c1] | −6.8 |
| 137 | 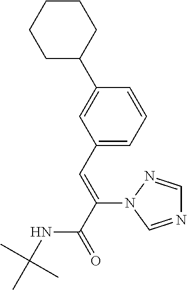 [SMILES: CC(C)(C)NC(=O)/C(=C/c1cccc(C2CCCCC2)c1)n1cncn1] | −6.8 |
| 138 | 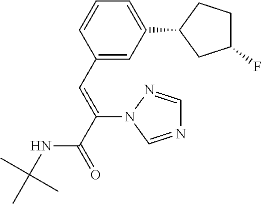 [SMILES: CC(C)(C)NC(=O)/C(=C/c1cccc([C@@H]2CC[C@H](F)C2)c1)n1cncn1] | −6.8 |
| 139 | 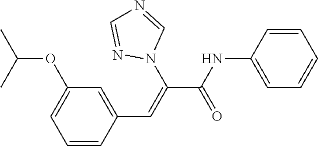 [SMILES: CC(C)Oc1cccc(/C=C(/C(=O)Nc2ccccc2)n2cncn2)c1] | −6.7 |
| 140 | 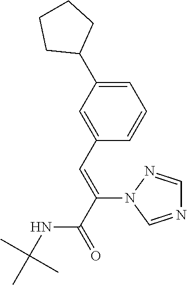 [SMILES: CC(C)(C)NC(=O)/C(=C/c1cccc(C2CCCC2)c1)n1cncn1] | −6.7 |
| 141 | 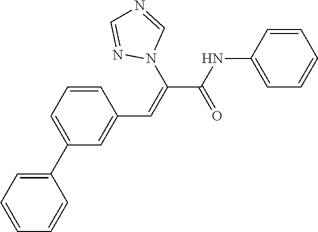 [SMILES: O=C(Nc1ccccc1)/C(=C/c1cccc(-c2ccccc2)c1)n1cncn1] | −6.6 |
| 142 | 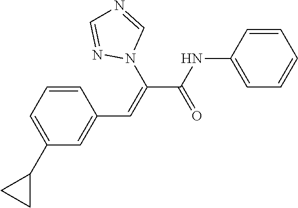 [SMILES: O=C(Nc1ccccc1)/C(=C/c1cccc(C2CC2)c1)n1cncn1] | −6.6 |
| 143 | 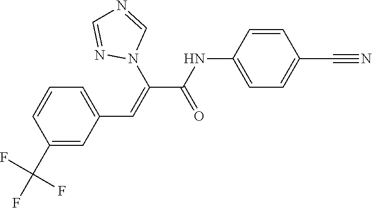 [SMILES: N#Cc1ccc(NC(=O)/C(=C/c2cccc(C(F)(F)F)c2)n2cncn2)cc1] | −6.6 |
| 144 | 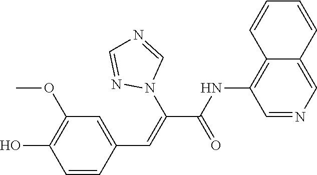 [SMILES: COc1cc(/C=C(/C(=O)Nc2cncc3ccccc23)n2cncn2)ccc1O] | −6.6 |
| 145 | 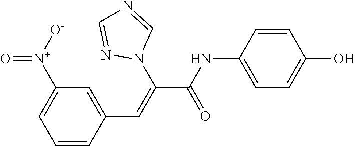 [SMILES: O=C(Nc1ccc(O)cc1)/C(=C/c1cccc([N+](=O)[O-])c1)n1cncn1] | −6.5 |
| 146 | 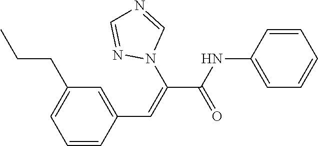 [SMILES: CCCc1cccc(/C=C(/C(=O)Nc2ccccc2)n2cncn2)c1] | −6.5 |
| 147 | 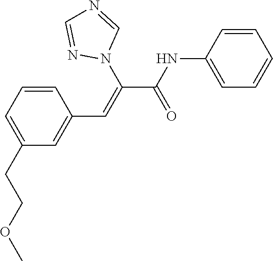 [SMILES: COCCc1cccc(/C=C(/C(=O)Nc2ccccc2)n2cncn2)c1] | −6.5 |
| 148 | 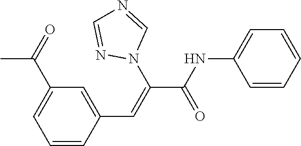 [SMILES: CC(=O)c1cccc(/C=C(/C(=O)Nc2ccccc2)n2cncn2)c1] | −6.5 |
| 149 | 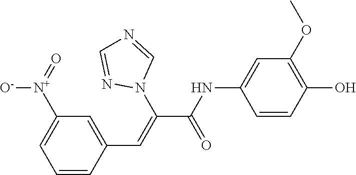 [SMILES: COc1cc(NC(=O)/C(=C/c2cccc([N+](=O)[O-])c2)n2cncn2)ccc1O] | −6.5 |
| 150 (C1) | 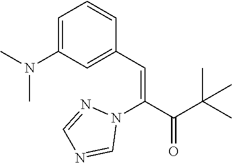 [SMILES: CN(C)c1cccc(/C=C(/C(=O)C(C)(C)C)n2cncn2)c1] | −5.4 |
| 151 (C2) | 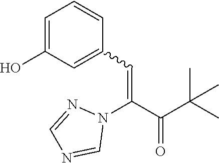 [SMILES: CC(C)(C)C(=O)C(=Cc1cccc(O)c1)n1cncn1] | −5.3 |
| 152 (C4) | 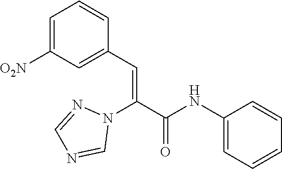 [SMILES: O=C(Nc1ccccc1)/C(=C/c1cccc([N+](=O)[O-])c1)n1cncn1] | −6.3 |
| 153 (C5) | 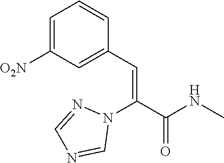 [SMILES: CNC(=O)/C(=C/c1cccc([N+](=O)[O-])c1)n1cncn1] | −5.4 |
| 154 (C6) | 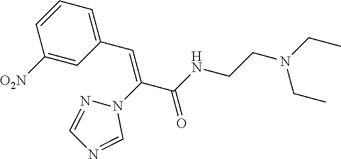 [SMILES: CCN(CC)CCNC(=O)/C(=C/c1cccc([N+](=O)[O-])c1)n1cncn1] | −5.2 |

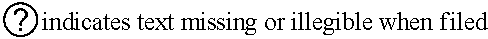

### Pharmaceutical Composition

The disclosure also provides a pharmaceutical composition comprising a therapeutically effective amount of one or more Inhibitors as described herein, or a pharmaceutically acceptable salt thereof, in admixture with a pharmaceutically acceptable carrier. In some embodiments, the pharmaceutical composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1, or a pharmaceutically acceptable salt thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of an Inhibitor or a pharmaceutically acceptable salt, stereoisomer, and/or tautomer thereof that is administered is governed by such considerations, and it is the minimum amount necessary to inhibit the interaction between the small GTPase Rab27a and its effector JFC1, inhibit the exocytosis of specific and/or azurophilic granules from neutrophils, reduce a neutrophil inflammatory response, reduce tissue infiltration by neutrophils, and combinations thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of an Inhibitor (or a pharmaceutically acceptable salt, stereoisomer, or tautomer thereof) of the present disclosure. In yet another embodiment, such dosage forms contain from about 25 mg to about 200 mg of an Inhibitor (or a pharmaceutically acceptable salt, stereoisomer, or tautomer thereof) of the present disclosure. In still another embodiment, such dosage forms contain from about 10 mg to about 100 mg of an Inhibitor (or a pharmaceutically acceptable salt, stereoisomer, or tautomer thereof) of the present disclosure. In a further embodiment, such dosage forms contain from about 5 mg to about 50 mg of an Inhibitor (or a pharmaceutically acceptable salt, stereoisomer, or tautomer thereof) of the present disclosure. In any of the foregoing embodiments the dosage form can be administered once a day or twice per day.

The Inhibitors of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The Inhibitors of the present disclosure that are suitable for oral use may be prepared according to any method known in the art for the manufacture of pharmaceutical compositions. For instance, liquid formulations of the Inhibitors of the present disclosure can contain one or more agents selected from the group consisting of sweetening agents, flavoring agents, coloring agents and preserving agents in order to provide pharmaceutically palatable preparations of the Inhibitors of the present disclosure.

For tablet compositions, the Inhibitors of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the Inhibitors of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the Inhibitors of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide Inhibitors of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The Inhibitors as described herein may also be administered in the form of suppositories for rectal administration of the drug. The Inhibitors can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The Inhibitors described herein can be administered to treat subjects, such as animals in need of such treatment, or who may develop a need for such treatment. For example, the Inhibitors can reduce the incidence and severity of diseases associated with systemic neutrophil-dependent inflammation. Examples of neutrophil-dependent inflammation that can be treated include coronary artery disease, autoinflammatory disease, sepsis, arthritis, ischemia-reperfusion injury, acute lung injury, glomerulonephritis, autoimmune disorders, and cancer. In various embodiments, the subject is chosen from humans, domesticated animals, zoo animals, and experimental animals.

Administration of the Inhibitors described herein can reduce the exocytosis of specific and azurophilic granules from neutrophils by at least 10%, or at least 20%, or at least 25%, or at least 30%, or at least 35%, or at least 40%, or at least 45%, or at least 50%, or at least 55%, or at least 60%, or at least 65%, or at least 70%, or at least 80%, at least 90%, at least 92%, at least 95%, at least 97%, at least 98%, or at least 99%. In some embodiments, the Inhibitors described herein can reduce neutrophil exocytosis of specific and azurophilic granules by 100%.

In additional embodiments, the present disclosure provides a method of treating a disease in a subject suffering therefrom. The method comprises administering to the subject one or more Inhibitors or pharmaceutically acceptable salt thereof as described herein. In some embodiments, the disease is one mediated by neutrophil dysfunction. Specific examples of diseases include Coronary Artery Disease, Inflammatory Arthritis, Ischemia-Reperfusion Injury, Autoimmune Disease, Sepsis, Infectious colitis, Ulcerative colitis, Crohn's disease, Ischemic colitis, Radiation colitis, Peptic ulcer, Intestinal, Breast and Ovarian cancer, tumor metastasis (wherein the tumorous disease includes skin squamous cell carcinoma, melanoma, adenocarcinomas, head and neck squamous cell carcinomas (HNSCC), and breast cancer), Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Left-sided heart failure, Right-sided heart failure, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schonlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Pressure Ulcers, Blepharitis, Furunculosis, Full or partial thickness burns, Capillaritis, Cellulitis, Corneal Abrasion, Corneal Erosion, Xerosis, Lichen Planus, Lichen Simplex Chronicus, Venous Ulcer (Stasis Ulcer), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease (ALD), Autoimmune Hepatitis, Biliary Atresia, Cirrhosis, Lysosomal Acid Lipase Deficiency (LAL-D), Lysosomal storage disorders, Liver Cysts, Liver Cancer, Newborn Jaundice, Non-Alcoholic Fatty Liver Disease, Non-Alcoholic Steatohepatitis, Primary Biliary Cholangitis (PBC), Progressive Familial Intrahepatic Cholestasis (PFIC), Osteoporosis, Paget's Disease, Osteonecrosis, Osteoarthritis, Low Bone Density, Gout, Fibrous Dysplasia, Marfan Syndrome, and Osteogenesis Imperfecta.

### EXAMPLES

Additional embodiments of the disclosure reside in specific examples and data described in more detail herein.

### Example 1. Formula (I) Inhibitors

### A. Synthesis

### General

The compounds used in the synthetic chemistry reactions described herein are made according to organic synthesis techniques known to those skilled in this art, starting from commercially available chemicals and/or from compounds described in the chemical literature. Commercially available chemicals are obtained from standard commercial sources.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2^{nd} Ed., Academic Press, New York, 1983; H. O. House, “Modern Synthetic Reactions”, 2^{nd} Ed., W. A. Benjamin, Inc. Menlo Park, Calif. 1972; T. L. Gilchrist, “Heterocyclic Chemistry”, 2^{nd} Ed., John Wiley & Sons, New York, 1992; J. March, “Advanced Organic Chemistry: Reactions, Mechanisms and Structure”, 4^{th} Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2^{nd} Edition (1999) Wiley-VCH, ISBN: 0-471-19031-4; March, J. “Advanced Organic Chemistry: Reactions, Mechanisms, and Structure” 4^{th} Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7^{th} Edition (2000) John Wiley & Sons, ISBN: 0-471-19095-0; Stowell, J. C., “Intermediate Organic Chemistry” 2^{nd} Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### General Synthetic Schemes

As used below and throughout the present disclosure, the following abbreviations, unless otherwise indicated, shall be understood to have the following meanings:

| | |
|---|---|
| °C | degrees Celsius |
| DMF | dimethylformamide |
| DIPEA | diisopropylethylamine |
| EDCL | 1-Ethyl-3-(3-dimethylaminopropyl)carbodiimide |
| HPLC | high performance liquid chromatography |
| LCMS | liquid chromatography mass spectrometry |
| HOBt | hydroxybenzotriazole |
| h | hour(s) |
| THF | tetrahydrofuran |

The compounds disclosed herein are prepared by a variety of synthetic routes including, but not limited to, the routes described below in Schemes 1 and 2.

Inhibitors C1 (Table 1, Inhibitor No. 150) and C2 (Table 1, Inhibitor No. 151) were prepared by the procedures illustrated in Scheme 1.

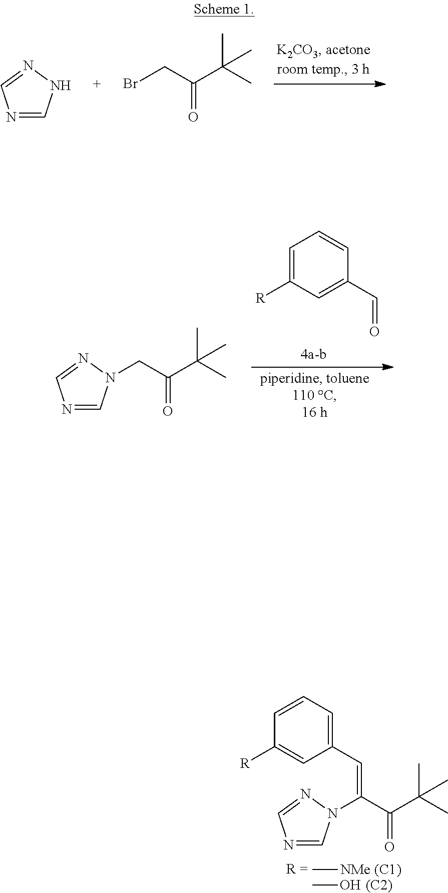

Inhibitors C4 (Table 1, Inhibitor No. 152), C5 (Table 1, Inhibitor No. 153), and C6 (Table 1, Inhibitor No. 154) were prepared by the procedures illustrated in Scheme 2.

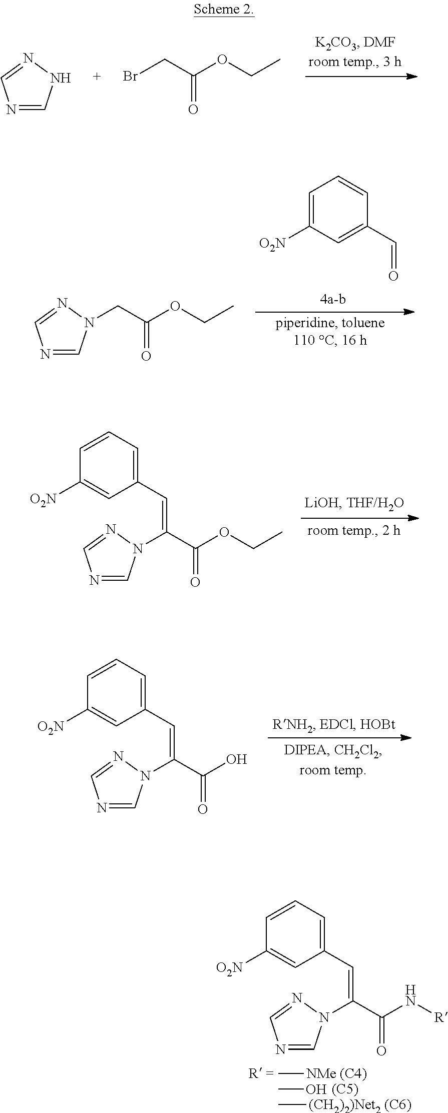

Final compounds prepared by the procedures above were purified by reverse phase HPLC and characterized by ¹H-NMR and LCMS. Select characterizing data is as follows:

| Inhibitor | LCMS [M + H] |
|---|---|
| C1 (150) | 299 |
| C2 (151) | 272 |
| C4 (152) | 336 |
| C5 (153) | 274 |
| C6 (154) | 359 |

### B. Screen and Docking Analysis

Using Chemdraw Pro v20.1.1.125 (PerkinElmer Informatic) approximately 2487 analogs were generated by fragment substitution at site R1 on the seed structure shown below and virtually screened against Rab27a:

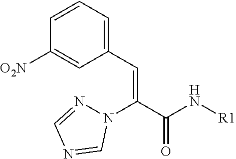

The ligand binding coordinate was taken as x=26.918748, y=16.706421 & z=−20.318112. The coordinate was calculated by using Discovery studio by highlighting binding site residues as previously reported (Johnson, et. al., Identification of Neutrophil Exocytosis Inhibitors (Nexinhibs), Small Molecule Inhibitors of Neutrophil Exocytosis and Inflammation, J. Bio. Chem. 291 (50): 25965-82 (2016), which is incorporated herein by reference in its entirety). The 154 resulting structures from the screen were further screened for Unfavorable Absorption, Distribution, Metabolism, and Elimination (ADME) properties. The data indicated that the identified structures follow Lipinski's rule of five and showed no PAINS alert. The data also indicated that the identified fragments and their substitution at R¹ position can increase binding affinity and inhibition potential of Nexinhib20 against Rab27a-JFC1 (PDB: 3BC1).

The Inhibitors described herein can reduce or prevent inflammation without affecting the initiation of the innate immune response, lack potentially pharmacologically toxic groups, and are optimized for drug likeness and oral bioavailability by filtering potential compounds to comply with at least four out of five of Lipinski's Rule of Five (RO5) (18):
(1) Molecular weight (MW) should be less than 500
(2) Hydrogen bond donor (HBD) should not exceed 5
(3) Hydrogen bond acceptor (HBA) should not exceed 10
(4) Log P i.e., an octanol-water partition coefficient does not exceed 5
(5) Polar surface area (PSA) should be below 140A².

A library of Inhibitors was filtered through a rule of three (MW<300, HBD≤3, HBA≤3 & PSA≤60 Δ²) so that the Inhibitors do not violate more than one RO5 rule. The Inhibitors were also screened for Pan-assay interference compounds (PAINS) alert (19). In drug discovery, the PAINS parameter can be used to eliminate chemical compounds that non-specifically bind to numerous biological targets.

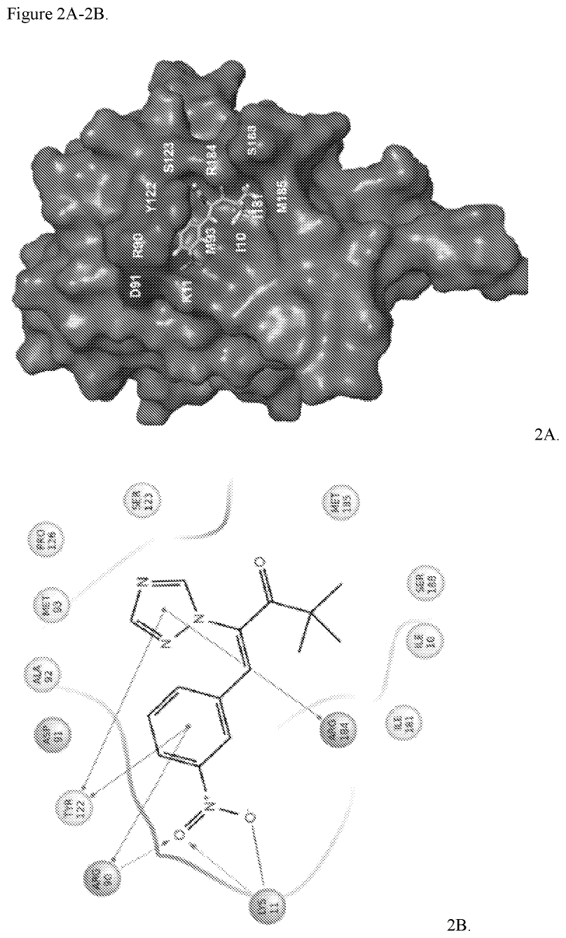

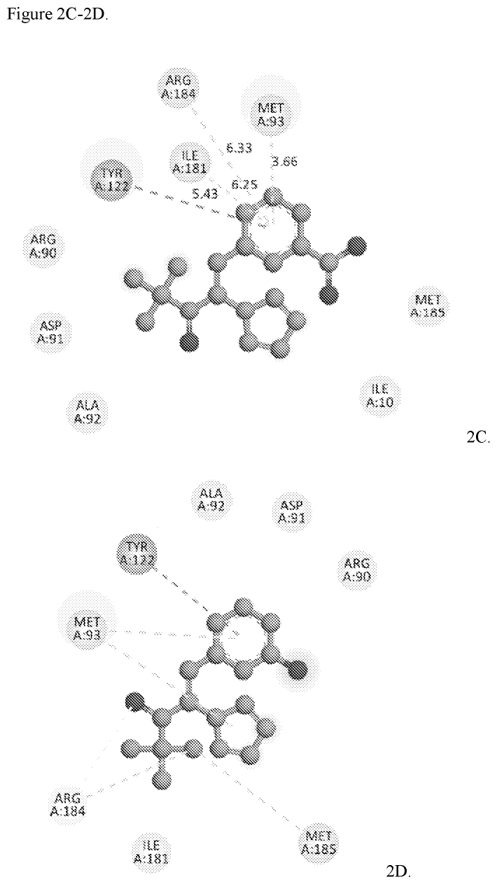

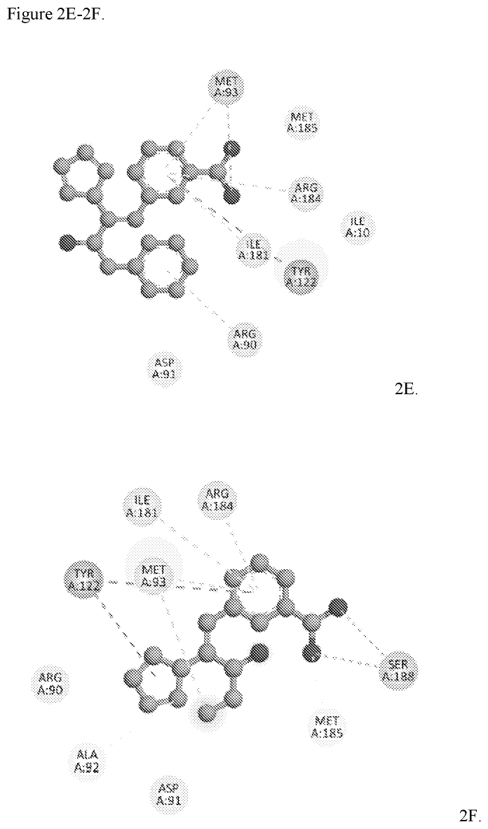

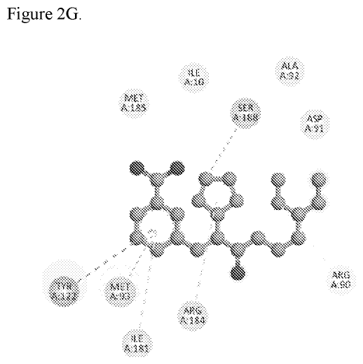

A molecular docking analysis of Nexinhib20 onto Rab27a was performed using Sitemap and Glide (Schrödinger, LLC, New York) as described previously (Johnson (2016)). A binding pocket for Nexinhib20 with the highest druggability score was identified within a Rab27a (FIG. 2A), which identified Tyr-122 in Rab27a as an important amino acid residue present in the Rab27a pocket that mediates π-π stacking interactions with Nexinhib20 (FIG. 2B). Importantly, structural studies identified Tyr-122 as a key residue for the Rab27a effector selectivity (21). In this way, point mutation of Tyr-122 disrupts the complex between Rab27a and Slp2a (21), which contains a Rab-binding domain highly similar to Slp1 (JFC1) (22). Molecular docking analysis further indicated that MET-93 and SER-188 are also key amino acids in addition to TYR-122 for interaction within the Rab27a binding pocket. Thus, a mechanism of action of the small molecule inhibitors through the occupation of a druggable pocket in Rab27a was identified.

Molecular docking analysis was performed on Inhibitors C1 (Table 1 Inhibitor No. 150), C2 (Table 1 Inhibitor No. 151), C4 (Table 1 Inhibitor No. 152), C5 (Table 1 Inhibitor No. 153), and C6 (Table 1 Inhibitor No. 154) (FIGS. 2C-G). FIG. 2C shows the molecular docking analysis of Inhibitor C1. TYR-122 and MET-93 of Rab27a interacts with an aromatic ring of C1. FIG. 2D shows the molecular docking analysis of Inhibitor C2. TYR-122 and MET-93 of Rab27a interacts with an aromatic ring of C2. FIG. 2E shows the molecular docking analysis of Inhibitor C4. C4 has an aromatic ring substitution which helps make a π-alkyl bond with Rab27a binding pocket amino acid ARG-90. TYR-122 and MET-93 of Rab27a also interacts with an aromatic ring of C4. FIG. 2F shows the molecular docking analysis of Inhibitor C5. TYR-122 and MET-93 of Rab27a interacts with an aromatic ring of C5. FIG. 2G shows the molecular docking analysis of Inhibitor C6. TYR-122 and MET-93 of Rab27a interacts with an aromatic ring of C6. SER-188 additionally interacts with the heteroaromatic ring of C6.

As shown in Table I, Inhibitors C1, C2, C4, C5, and C6 have Rab27a binding pocket binding energies of −5.4, −5.3, −6.3, −5.4, and −5.2 kcal/mol, respectively. Inhibitor C4 has the markedly lower binding energy of −6.3 kcal/mol as compared to the other Inhibitors C1, C2, C5, and C6 owing to its aromatic substitution and π-alkyl bond with Rab27a binding pocket amino acid ARG-90. Table I includes the results of the screening for additional Inhibitors 1-149 that have an improved binding energy of less than or equal to a threshold value of −6.5 kcal/mol.

### Example 2. Inhibition of Neutrophil Exocytosis by Inhibitors C1, C2, C4, C5, and C6

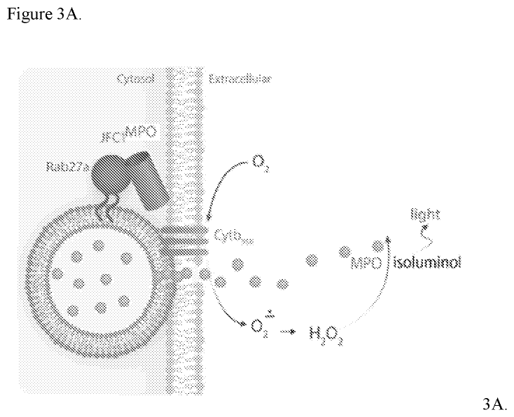

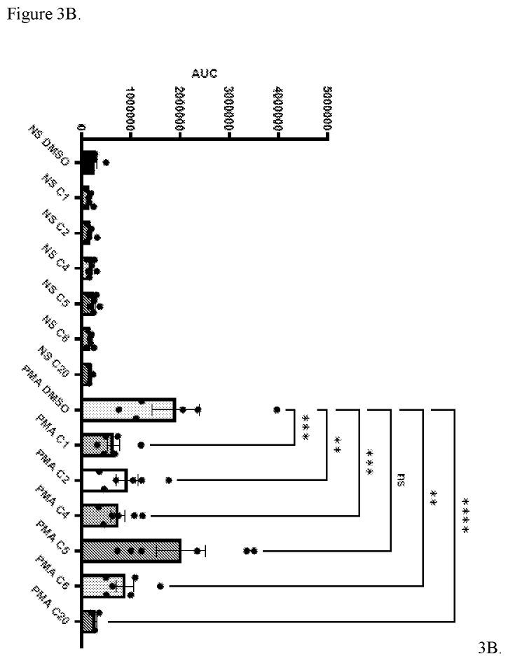

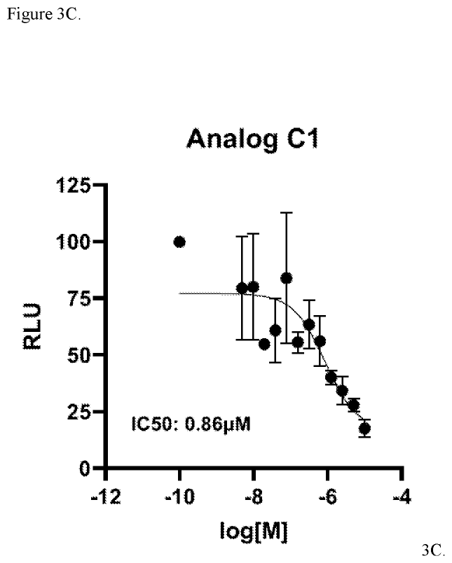

FIGS. 3A-C illustrate the effect of Inhibitors C1, C2, C4, C5, and C6 and comparator Nexinhib 20 (shown as “C20”) on MPO secretion of mouse neutrophils. FIG. 3A is a schematic representation of a cell-based secondary screening assay used to analyze the potency the potency of Rab27a-JFC1 inhibitors for their ability to decrease exocytosis of intact human neutrophil. This cell-based assay measures the secretion of myeloperoxidase (MPO) by human neutrophils using cell-impermeant isoluminol-dependent chemiluminescence in the absence of exogenous peroxidases.

Methods: Neutrophil Isolation and Secondary Cell-based Chemiluminescence Assay and Neutrophil Secretion Assays

Human neutrophils were isolated from normal donor's blood by Ficoll density centrifugation, as described previously (23). Murine bone marrow-derived neutrophils were isolated using a Percoll gradient fractionation system as described (24). A three-layer Percoll gradient was used (52%, 64%, and 72%), and neutrophils were isolated from the 64-72% interface, washed, and used in the assays.

Extracellular MPO-dependent reactive oxygen species (ROS) production was measured using the chemiluminescence reactions mediated by isoluminol (25). To this end, 3×10⁵ neutrophils were resuspended in serum-free RPMI 1640 medium in the presence of cell-impermeant isoluminol, and reactions were carried out in the absence of exogenous peroxidase. Neutrophils were incubated in the presence of compounds or DMSO (0.5%) pin-tooled into 384-well plates in a 40-1 volume. Control experiments were run in parallel in the presence of sodium azide (0.5 mM) to inhibit endogenous myeloperoxidase. After addition of stimuli (phorbol 12-myristate 13-acetate, “PMA”) or vehicle using liquid handling devices (BioRaptor FRD, Beckman Coulter), MPO-dependent chemiluminescence was continuously monitored for 30 min at 37 °C using a 2104 EnVision multilabel plate reader. For secretion assays, 0.5×10⁶ neutrophils were incubated in RPMI 1640 medium in polystyrene 96-well plates in the presence of Inhibitors or dimethyl sulfoxide (DMSO) control for 1 hour and stimulated for the indicated times at 37 °C with PMA (0.1 μg/ml), GM-CSF (10 ng/ml), and fMLP (1 μM) or DMSO control. The cells were spun down, and supernatants were transferred to clean plates using plate filters to avoid contamination from cellular secretory proteins. MPO was measured by ELISA (R&D Systems).

Results: FIG. 3B is a graph showing the effect of Inhibitors C1, C2, C4, C5, and C6 on secreted MPO-dependent reaction using the isoluminol-dependent chemiluminescence assay in highly purified mouse neutrophils. On the graph of FIG. 3B, the pre-fix “NS” means “no stimuli” where the cells were treated with 10 μM C1, C2, C4, C5, C6, and C20 alone without any exocytosis stimuli. The pre-fix “PMA” means the cells were stimulated with PMA to secrete MPO and were treated with 10 μM of the designated Inhibitor. DMSO was used as a control instead of an Inhibitor and shows the amount of secreted MPO in cells not treated with any Inhibitor. Inhibitors C1, C2, C4, and C6, show significant inhibition of exocytosis by the neutrophils as compared to the DMSO control. Mean % inhibition: C1, 76.74; C2, 59.85; C4, 70.9; C6, 62.4; C20, 99.22.

FIG. 3C is a graph of a dose-response analysis of Inhibitor C1 using the chemiluminescence-based MPO secretion assay. The graph of FIG. 3C shows a decreasing isoluminol-dependent signal with calculated IC50 value of 0.86 μM indicating that the decrease in exocytosis is a function of treatment with increasing amounts of C1.

### Example 3: Effect of Small Molecule Inhibitors on Exocytosis in Human Neutrophils

Method: The up-regulation of granule membrane-associated neutrophil markers at the plasma membrane was analyzed by flow cytometry. In these studies, flow cytometry analysis was performed by resuspension of 1×10⁶ human neutrophils in phenol red-free RPMI 1640 medium, treated with a 10 μM solution of Inhibitors C1, C2, C4, C5, and C6 and Nexinhib 20 (C20) or DMSO control for 1 hour, and stimulated with physiological agonists GM-CSF (10 ng/ml) for 30 min followed by the addition of fMLP (1 μM) to induce degranulation or left untreated at 37 °C (non-stimulated, NS). The reactions were stopped by transferring the samples to ice and immediately spinning down the cells to initiate blocking and staining. To analyze the plasma membrane expression of plasma membrane markers, the cells were blocked in ice-cold PBS containing 1% BSA and stained with phycoerythrin (PE)-, FITC-, or Alexa647-conjugated anti-human CD63, CD66b, MMP9, and CD11b antibodies that detect extracellular epitopes of these markers. The cells were then washed and fixed in 1% paraformaldehyde in PBS. Samples were analyzed using a NovoCyte flow cytometer, and the data were processed using FlowJo software.

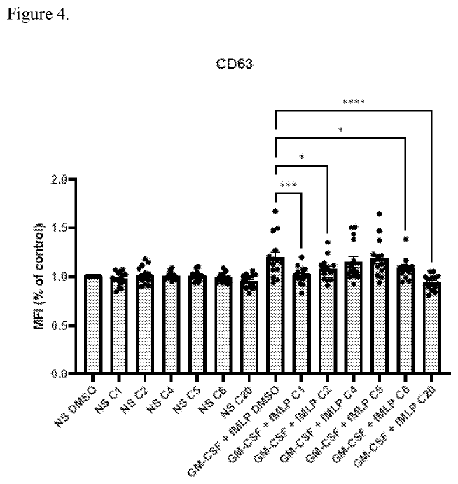

Results: FIG. 4 is a graph of flow cytometry analysis showing the effect of the Inhibitors C1, C2, C4, C5, and C6 on the mobilization of azurophilic granule marker CD63. The DMSO control group treated with GM-CSF and fMLP to induce degranulation showed the reference amount of CD63 up-regulation. Treatment of the cells with Inhibitors C1 and C2 and with C20 showed marked and significant inhibition of CD63 up-regulation relative to the DMSO control. Mean % inhibition: C1, 93.12%; C2, 59.53%; C6, 58.06%. ANOVA (Sidak's multiple comparisons test). N=15 individual donors.

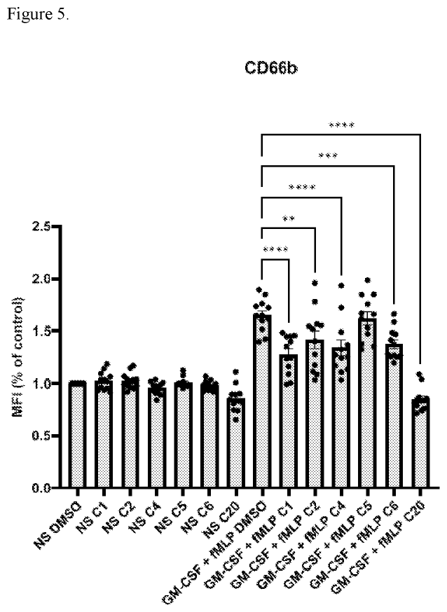

FIG. 5 is a graph of flow cytometry analysis showing the effect of the Inhibitors C1, C2, C4, C5, and C6 on the mobilization of specific granule marker CD66b. Treatment of neutrophils with C1, C2, C4, and C6 and with C20 reduced the up-regulation of CD66b. The DMSO control group treated with GM-CSF and fMLP to induce degranulation showed the reference amount of CD66b up-regulation. Treatment of the cells with C1, C2, C4, and C20 all showed marked and significant inhibition of CD66b up-regulation relative to the DMSO control. % inhibition: C1, 57.56; C2, 35.81; C4, 48.14; C6 42.33% and C20 100% ANOVA (Sidak's multiple comparisons test). N=12 individual donors.

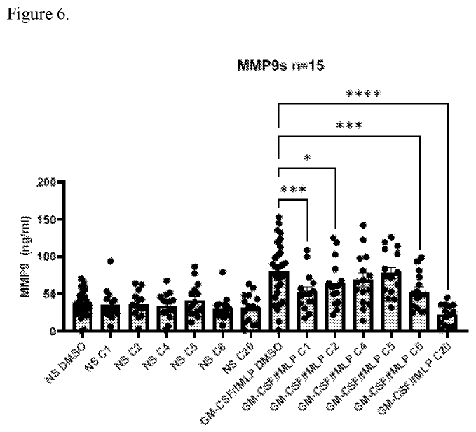

FIG. 6 is a graph of flow cytometry analysis showing the effect of the Inhibitors C1, C2, C4, C5, and C6 on the mobilization of Gelatinase granule cargo MMP9. The human neutrophils in the DMSO control group treated with GM-CSF and fMLP to induce degranulation showed the reference amount of MMP9 up-regulation. Treatment of the human neutrophils with Inhibitors C1, C2, and C6 and with C20 all showed marked inhibition of CD66b up-regulation relative to the DMSO control. % Inhibition: C1, 55.27%; C2, 26.5; C4, 16.45; C6, 54.4; ANOVA (Sidak's multiple comparisons test). N=12 individual donors.

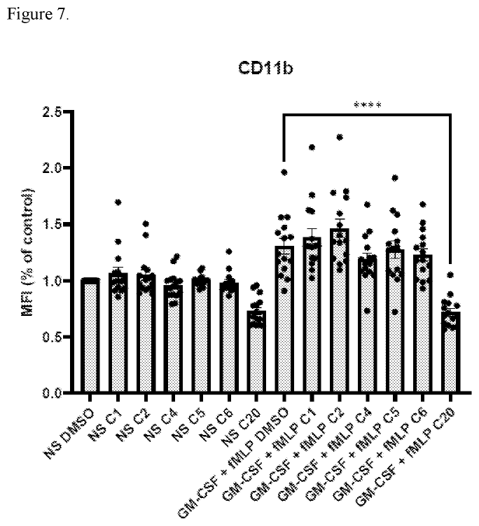

FIG. 7 is a graph of flow cytometry analysis showing the effect of the Inhibitors C1, C2, C4, C5, and C6 and also C20 on the mobilization of secretory vesicles by detecting secretory vesicle marker CD11b. CD11b is an adhesion molecule and CD11b+ vesicles are readily mobilizable in human polymorphonuclear neutrophils (hPMN). CD11b+ vesicles drive the initiation of the innate immune response. The human neutrophils in the DMSO control group treated with GM-CSF and fMLP show the reference amount of CD11b+ vesicle release. Treatment of the human neutrophils with Inhibitors C1, C2, C4, C5, and C6 show the release of CD11b+ vesicles is unaffected by the small molecule inhibitors relative to the DMSO control. The lack of activity of Inhibitors C1, C2, C4, C5, and C6 on CD11b+ vesicles indicates that the Inhibitors can prevent inflammation without affecting the initiation of the innate immune response. Treatment of human neutrophils with Nexinhib 20 (C20) show an inhibition of CD11b+ vesicle release, indicating that C20 can disrupt the early steps in initiation of the neutrophil innate immune response. ANOVA (Sidak's multiple comparisons test). N=15 individual donors. Mean % Inhibition: only C20 significantly inhibited CD11b upregulation at the plasma membrane (to levels lower than basal controls).

### Analysis of the Effect of New Neutrophil-Exocytosis Inhibitors in Models of Systemic Inflammation and Autoinflammatory Disease

Systemic inflammatory syndromes caused by either genetic defects, exacerbated innate immune responses to infection, trauma or autoimmune disorders are characterized by elevated plasma levels of pro-inflammatory cytokines and by the dysregulated and deleterious activation of the innate immune system, but inhibitors of neutrophil-mediated systemic inflammation are lacking. The NLRP3 inflammasome has been implicated in numerous common inflammatory disorders (e.g., gout, atherosclerosis, reumathoid arthritis, coronary artery disease and cancer) (Mol Cell 10, 417-426 (2002); Nature 464, 1357-1361, doi:10.1038/nature08938 (2010)) as well as normal and dysregulated host response to infection (e.g., Streptococcus aureus and malaria). (J Immunol 183, 5823-5829, doi:10.4049/jimmunol.0900444 (2009); PLoS pathogens 5, e1000559, doi:10.1371/journal.ppat.1000559 (2009)).

### Analysis of the Effect of New Neutrophil-Exocytosis Inhibitors on Neutrophil Secretion Using Modes of Systemic Inflammation and Autoinflammatory Disease

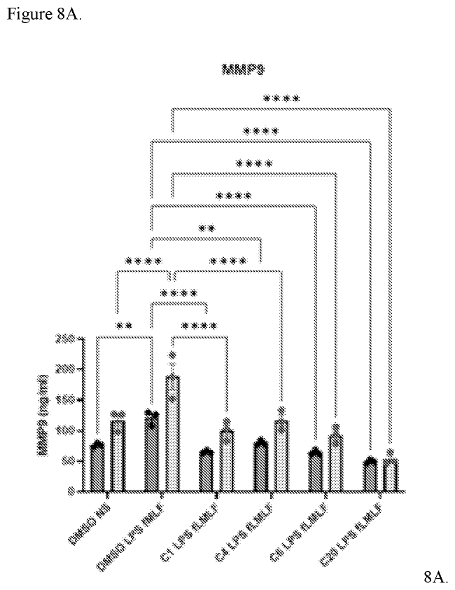

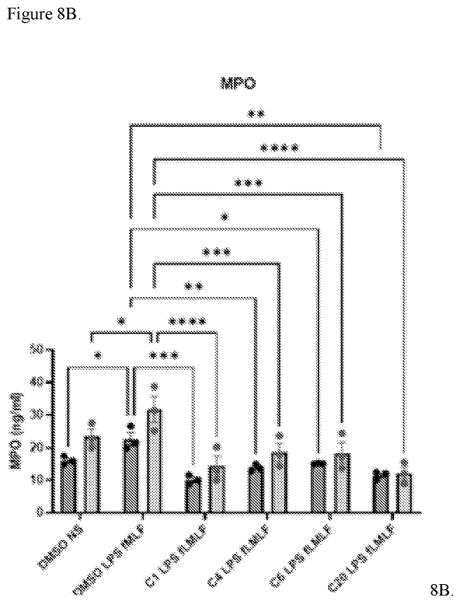

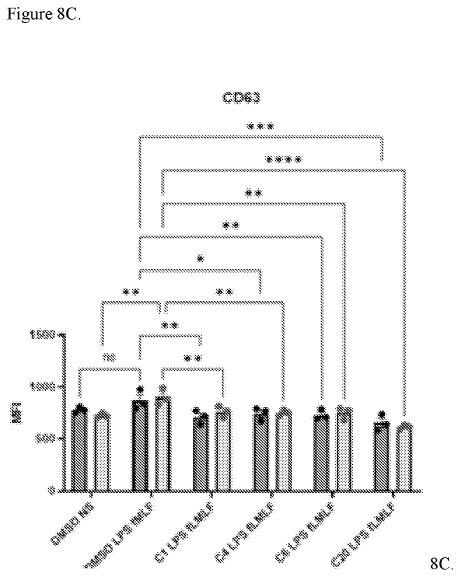

The new Nexinhib analogs (NEIa) C1, C2, C4 and C6 have a selective inhibitory effect on wild-type neutrophils (shown in the provisional filling). These compounds are now tested using neutrophils from a mouse model of Cryopyrin-Associated Periodic Syndrome (CAPS), a systemic inflammatory disease in humans, caused by gain-of-function mutations of the NOD-like receptor family member, cryopirin, also known as NLRP3 (J Cin Immunol 39, 277-286, doi:10.1007/s10875-019-00638-z (2019)). Heterozygous NLRP3 mutations lead to hyper-responsive NLRP3 inflammasome function characterized by increased caspase-1 (Casp1) activation and IL-10 production, and is observed in patients and in animal models with NLRP3-gain-of-function mutations. Using the Nlrp3^{A350V} (knockin) mouse model of CAPS (MWS CreT,) that phenocopies the disease in humans with the A352V mutation in NLRP3, we demonstrated that NLRP3 dysregulation leads to exacerbated azurophilic granule exocytosis (Johnson, Hoffman and Catz, Cell Infect Microbiol 7, 507, doi:10.3389/fcimb.2017.00507 (2017).). We now show that the new compounds NEIa-C1, C4-C6 significantly inhibit secretion not only in wild type cells but also in neutrophils that carry the NLRP3 A350V mutation (FIG. 8A-C). In these experiments, the effective inhibition of neutrophil exocytosis was demonstrated using Nexinhib analogs at 10 μM to treat bone-marrow-derived neutrophils for 1 hour. Subsequently, neutrophils were either stimulated with LPS and the chemotatctic peptide fMLF or left untreated. In FIG. 8, we show that NEIaC1, C4 and C6 inhibit the mobilization of gelatinase granules (MMP9), azurophilic granules marked with the cargo myeloperoxidase (MPO). The inhibitors also significantly inhibited the mobilization of CD63, an azurophilic and endolysosomal marker in murine neutrophils.

### In Vivo Analysis of the Effect of New Neutrophil-Exocytosis Inhibitors on Neutrophil-Mediated Inflammation in a Model of Autoinflammatory Disease

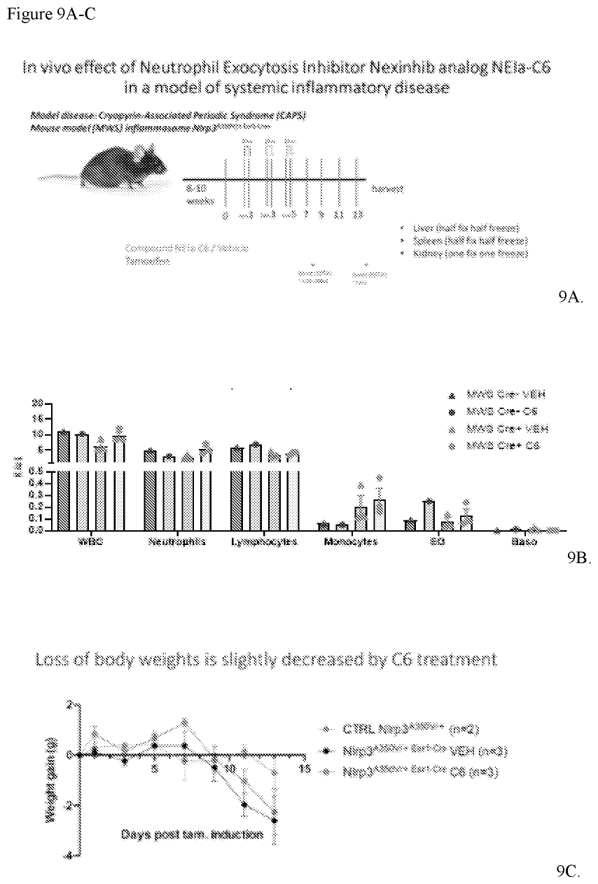

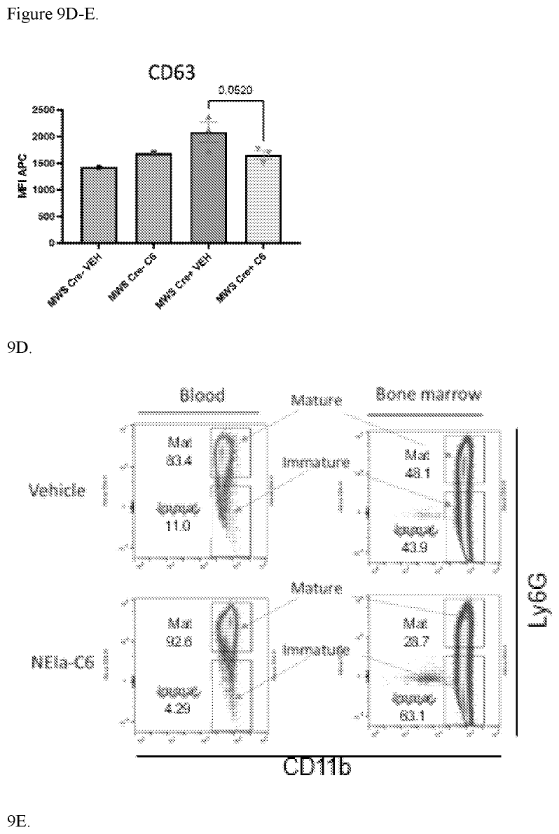

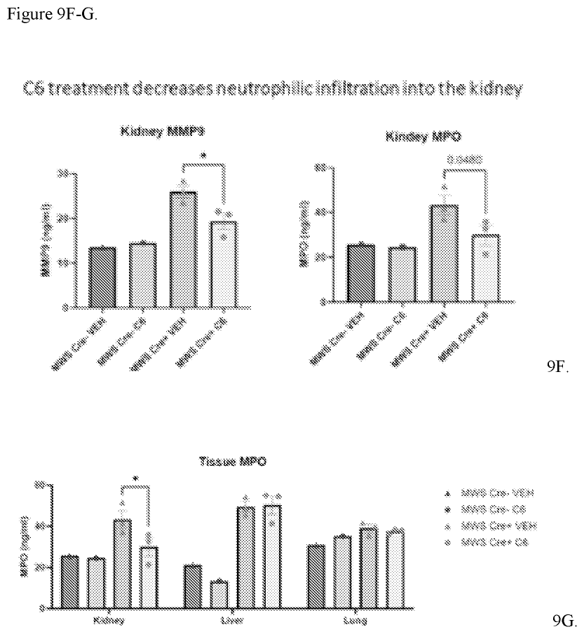

Next, to analyze whether the chemically improved new small-molecules were also effective in vivo, we tested NEIa-C6 in a mouse model of the autoinflammatory disease CAPS (cryopyrin-associated periodic syndrome). In this model, disease is caused by the Nlrp3^{A350V} mutations. The Nlrp3^{A350V} inducible mouse model (MWS CreT) is homologous to humans suffering from CAPS with the Muckle-Wells A352V mutation in the NLRP3 gene. The tamoxifen inducible Nrp3^{A350V} cre ERT2 and control animals were injected (i.p.) with 50 mg/kg tamoxifen free base to induce disease (J Immunol 189, 2707-2711, doi:10.4049/jimmunol.1101737 (2012)). The mice (6-12 weeks old) had access to food and water ad libitum. All animal studies were performed in compliance with the U.S. Department of Health and Human Services Guide for the Care and Use of Laboratory Animals and according to NIH and institutional guidelines. All animal protocols and procedures were approved by the University of California San Diego Institutional Animal Care and Use Committees (IACUC). Mice were treated with the neutrophil specific inhibitor NEIa-C6 or vehicle (5% DMSO in PBS). In this model, NLRP3^{A350V} disease develops progressively in 15 days after first induction with tamoxifen. The experimental design is presented in FIG. 9A. Between day 0 and day 5 mice were injected (i.p) every other day with tamoxifen followed by NEIa-C6 (30 mg/Kg) or vehicle. Next, administration of NEIa-C6 or vehicle continued until day 13, in the absence of tamoxifen. Blood samples were obtained at day 7 and blood and tissues harvested at day 13. Treatment with NEIa-C6 did not affect the number of any leukocyte subtype at 7 or 13 days (FIG. 9B and data not shown). Animals treated with NEIa-C6 were partially protected from weight loss compared to vehicle treated controls (FIG. 9C). Remarkably, NEIa-C6 decreased neutrophil activation in vivo, manifested as the decreased upregulation of the azurophilic and endolysosomal marker CD63 (FIG. 9D). Importantly, treatment with NEIa-C6 also decreased the number of pro-inflammatory immature neutrophils in blood (FIG. 9E). Finally, treatment with NEIa-C6 significantly protected kidneys from neutrophilic infiltration (FIG. 9F). The effect was specific for kidneys as lung and liver infiltration was unaffected (FIG. 9G).

### In Vivo Analysis of the Effect of New Neutrophil-Exocytosis Inhibitors on Neutrophil-Mediated Inflammation in a Model of Endotoxemia/Sepsis

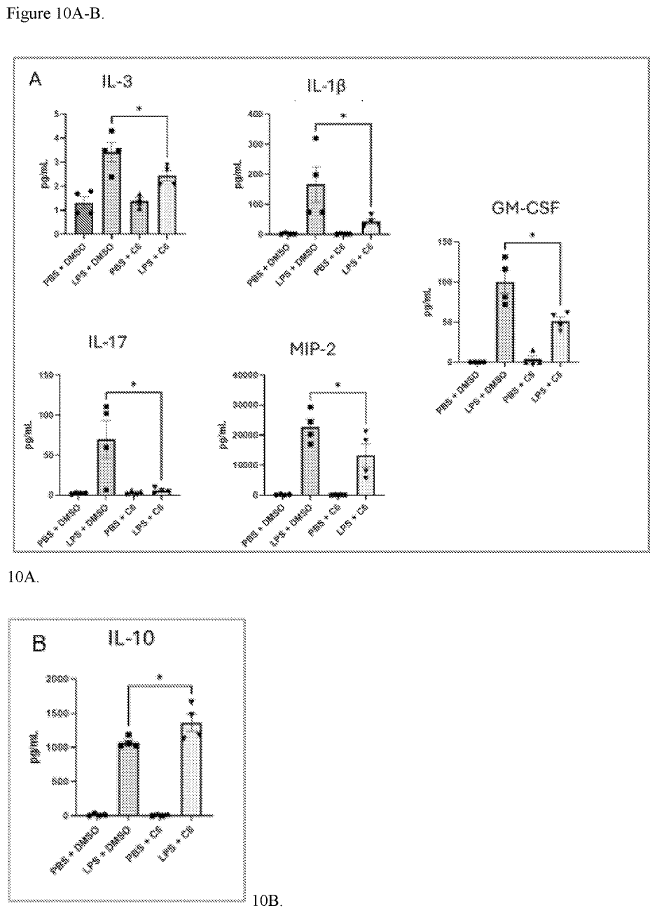

To test whether Nexinhibs, and in particular NEIa-C6, have additional protective properties in inflammation models relevant to human disease, independent of inflammasome activation, we tested the ability of C6 to protect mice from LPS-induced systemic inflammation. In these experiments, mice received NEIa-C6 (30 mg/Kg) or vehicle by a single i.p. injection and two hours later, the animals are challenged with a single i.p. injection of lipopolysaccharide (LPS, 8 mg/Kg), an important outer membrane component of gram-negative bacteria. In this model, endotoxemia insult induces the production of pro-inflammatory cytokines. To test the possible anti-inflammatory effect of NEIa-C6, we comparatively analyzed cytokine levels in plasma of LPS-insulted and control mice using multiplex technology. In FIG. 10, we show that NEIa-C6 treatment significantly decreases the production of pro-inflammatory cytokines IL-1β and GM-CSF (FIG. 10A). Notably, NEIa-C6 also decreases the levels of TL-3 (FIG. 10A), a pro inflammatory cytokine that stimulates neutrophils, amplifies acute inflammation and is a potential therapeutic target in sepsis (Science 347, 1260-1265, doi:10.1126/science.aaa4268 (2015)).

Of note, TL-3 deficiency protects mice against sepsis, highlighting NEIa-C6 as a possible agent to combat systemic inflammation in sepsis. NEIa-C6 also decreased the levels of MIP-2, an important neutrophil chemoattractant in mice, and IL-17 (FIG. 10A), a mediator of neutrophilic kidney infiltration and renal fibrosis (Am J Physiol Renal Physiol 312, F385-F397, doi:10.1152/ajprenal.00462.2016 (2017)). Also important, NEIa-C6 increased the levels of IL-10 which is anti-inflammatory and protective in sepsis (FIG. 10B). In fact, absence of IL-10 was shown to accelerate irreversible shock and increases pro-inflammatory cytokines 15 times in sepsis, while recombinant IL-10 significantly improves survival (Infection and immunity 70, 4441-4446, doi:10.1128/IAI.70.8.4441-4446.2002 (2002)), again, highlighting the multiple protective mechanisms mediated by NEIa-C6.

Development of New Analogs of Nexinbib with Substitutions in Positions C3 and C10

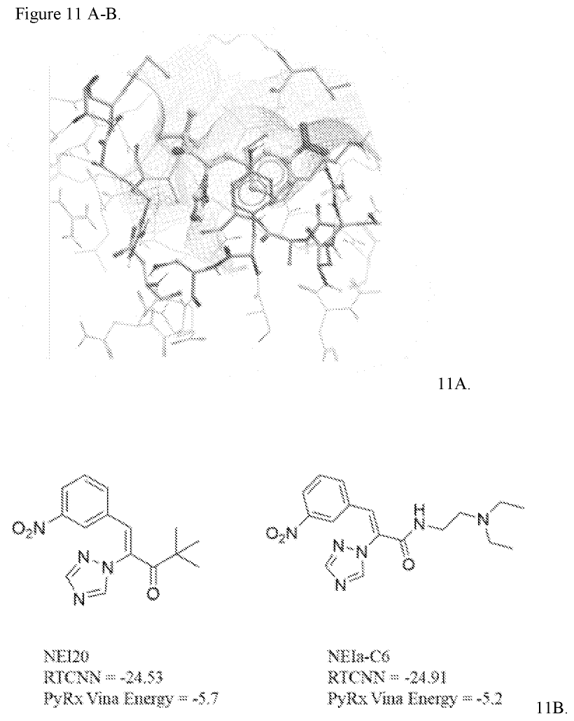

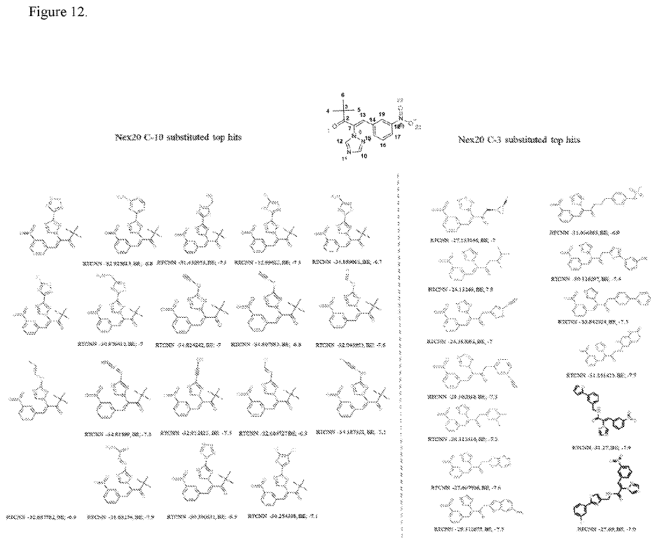

The representation of Nexinhib20 (NEI20) in the Nexinhib20-Rab27a binding pocket identified a relatively large space for fragment substitution and expansion associated with the Triazole group in Nexinhib20, while substitutions in position C3 may hinder with JFC1 binding, a property that was utilized in the design of NEIa-C6 (FIG. 11). We now applied cluster-based screening of ~120,000 newly designed molecules to segregate hits based on a double-score screening criteria consensus: a) Energy binding-based screening using PyRx and b) Topological (chemical graph) and 3D Radial convolutions screenings, i.e. RTCNN Score Neural Network score, using MolSoft. Using these double-score criteria consensus, and cutoffs equal or better than those shown by Nexinhib20 scores, we designed new analogs of Nexinhib20 with modifications in C3 and C10 (FIG. 12).

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

### REFERENCES

Numbered references in the present disclosure are as follows:
(1) Segal, A. W. How neutrophils kill microbes. Annu. Rev. Immunol. 23, 197-223 (2005).
(2) Vita, J. A., Brennan, M. L., Gokce, N., Mann, S. A., Goormastic, M., Shishehbor, M. H., Penn, M. S., Keaney, J. F., Jr., and Hazen, S. L. Serum myeloperoxidase levels independently predict endothelial dysfunction in humans. Circulation 110, 1134-1139 (2004).
(3) Kothari, N., Keshari, R. S., Bogra, J., Kohli, M., Abbas, H., Malik, A., Dikshit, M., and Barthwal, M. K. Increased myeloperoxidase enzyme activity in plasma is an indicator of inflammation and onset of sepsis. J. Crit. Care 26, 435.e1-7 (2011).
(4) Zheng, L., Nukuna, B., Brennan, M. L., Sun, M., Goormastic, M., Settle, M., Schmitt, D., Fu, X., Thomson, L., Fox, P. L., Ischiropoulos, H., Smith, J. D., Kinter, M., and Hazen, S. L. Apolipoprotein A-I is a selective target for myeloperoxidase-catalyzed oxidation and functional impairment in subjects with cardiovascular disease. J. Clin. Invest. 114, 529-541 (2004).
(5) Baldus, S., Heeschen, C., Meinertz, T., Zeiher, A. M., Eiserich, J. P., Mü\hat{n}zel, T., Simoons, M. L., Hamm, C. W., and CAPTURE Investigators Myeloperoxidase serum levels predict risk in patients with acute coronary syndromes. Circulation 108, 1440-1445 (2003).
(6) Moraes, T. J., Chow, C. W., and Downey, G. P. Proteases and lung injury. Crit. Care Med. 31, S189-S194 (2003).
(7) Brown, K. A., Brain, S. D., Pearson, J. D., Edgeworth, J. D., Lewis, S. M., and Treacher, D. F. Neutrophils in development of multiple organ failure in sepsis. Lancet 368, 157-169 (2006).
(8) Nuijens, J. H., Abbink, J. J., Wachtfogel, Y. T., Colman, R. W., Eerenberg, A. J., Dors, D., Kamp, A. J., Strack van Schijndel, R. J., Thijs, L. G., and Hack, C. E. Plasma elastase_1-antitrypsin and lactoferrin in sepsis: evidence for neutrophils as mediators in fatal sepsis. J. Lab. Clin. Med. 119, 159-168 (1992).
(9) Borregaard, N. Subcellular localization and dynamics of components of the respiratory burst oxidase. J. Bioenerg. Biomembr. 20, 637-651 (1988).
(10) Borregaard, N., and Cowland, J. B. Granules of the human neutrophilic polymorphonuclear leukocyte. Blood 89, 3503-3521 (1997).
(11) Monfregola, J., Johnson, J. L., Meijler, M. M., Napolitano, G., and Catz, S. D. (2012) MUNC13-4 protein regulates the oxidative response and is essential for phagosomal maturation and bacterial killing in neutrophils. J. Biol. Chem. 287, 44603-44618 (2012).
(12) Munafo, D. B., Johnson, J. L., Brzezinska, A. A., Ellis, B. A., Wood, M. R., and Catz, S. D. DNase I inhibits a late phase of reactive oxygen species production in neutrophils. J. Innate Immun. 1, 527-542 (2009).
(13) Johnson, J. L., Pacquelet, S., Lane, W. S., Eam, B., and Catz, S. D. Akt regulates the subcellular localization of the Rab27a-binding protein JFC1 by phosphorylation. Traffic 6, 667-681 (2005).
(14) Brzezinska, A. A., Johnson, J. L., Munafo, D. B., Crozat, K., Beutler, B., Kiosses, W. B., Ellis, B. A., and Catz, S. D. The Rab27a effectors JFC1/Slp1 and Munc13-4 regulate exocytosis of neutrophil granules. Traffic 9, 2151-2164 (2008).
(15) Johnson, J. L., Brzezinska, A. A., Tolmachova, T., Munafo, D. B., Ellis, B. A., Seabra, M. C., Hong, H., and Catz, S. D. (2010) Rab27a and Rab27b regulate neutrophil azurophilic granule exocytosis and NADPH oxidase activity by independent mechanisms. Traffic. 11, 533-547 (2010).
(16) Johnson, J. L., Hong, H., Monfregola, J., and Catz, S. D. Increased survival and reduced neutrophil infiltration of the liver in Rab27a- but not Munc13-4-deficient mice in lipopolysaccharide-induced systemic inflammation. Infect. Immun. 79, 3607-3618 (2011).
(17) Munafó, D. B., Johnson, J. L., Ellis, B. A., Rutschmann, S., Beutler, B., and Catz, S. D. Rab27a is a key component of the secretory machinery of azurophilic granules in granulocytes. Biochem. J. 402, 229-239 (2000).
(18) Lipinski C A. Drug-like properties and the causes of poor solubility and poor permeability. J. Pharmacology. Toxicol. Methods. 44: 235-249 (2000).
(19) Baell J. B.; Holloway G. A. New Substructure Filters for Removal of Pan Assay Interference Compounds (PAINS) from Screening Libraries and for Their Exclusion in Bioassays. J. Med. Chem. 53: 2719-2740 (2010).
(20) Munafo D. B., Rab27a is a key component of the secretory machinery of azurophilic granules in granulocytes. Biochem J. 402: 229-239 (2007).
(21) Chavas, L. M., Ihara, K., Kawasaki, M., Torii, S., Uejima, T., Kato, R., Izumi, T., and Wakatsuki, S. Elucidation of Rab27 recruitment by its effectors: structure of Rab27a bound to exophilin4/Slp2-a. Structure 16, 1468-1477 (2008).
(22) Kuroda, T. S., Fukuda, M., Ariga, H., and Mikoshiba, K. The Slp homology domain of synaptotagmin-like proteins 1-4 and Slac2 functions as a novel Rab27A binding domain. J. Biol. Chem. 277, 9212-9218 (2002).
(23) Markert, M., Andrews, P. C., and Babior, B. M. Measurement of O2-production by human neutrophils. The preparation and assay of NADPH oxidase-containing particles from human neutrophils. Methods Enzymol. 105, 358-365 (1984).
(24) Johnson, J. L., Monfregola, J., Napolitano, G., Kiosses, W. B., and Catz, S. D. Vesicular trafficking through cortical actin during exocytosis is regulated by the Rab27a effector JFC1/Slp1 and the RhoA-GTPase activating protein Gem-interacting protein. Mol. Biol. Cell 23, 1902-1916 (2012).
(25) Johnson, J. L., Brzezinska, A. A., Tolmachova, T., Munafo, D. B., Ellis, B. A., Seabra, M. C., Hong, H., and Catz, S. D. (2010) Rab27a and Rab27b regulate neutrophil azurophilic granule exocytosis and NADPH oxidase activity by independent mechanisms. Traffic 11, 533-547 (2010).

We claim:

1. A compound of Formula I or a pharmaceutically acceptable salt thereof: 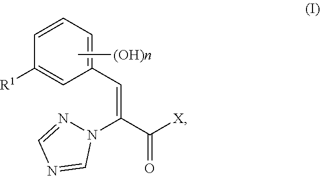
wherein
R¹ is selected from the group consisting of H, —NO₂, —OH, —C₁-C₆-alkyl, —C₁-C₆-haloalkyl, —C₁-C₆-alkyl-O—(C₁-C₆-alkyl), —C₁-C₆-alkenyl, —C₁-C₆-alkoxyl, —NRR′, —C(O)R, C₃-C₈-cycloalkyl, and —C₆-C₁₀-aryl;
wherein any alkyl, alkenyl, alkoxyl, and cycloalkyl is optionally substituted with 1 to 6 halo;
X is —NR^{X}(R²) or C₁-C₆-alkyl;
R^{X} is H or C₁-C₆-alkyl;
R² is selected from the group consisting of
Het, Cy,
—C(O)(Het), —C(O)(Cy),
—C(O)(NH)(Het), —C(O)(NH)(Cy),
—OC(O)(Het), —OC(O)(Cy),
—O(Het), —O(Cy),
—(C₁-C₆-alkyl)(Het), —(C₁-C₆-alkyl)(Cy),
—(C₁-C₆-hydroxyalkyl)(Het), —(C₁-C₆-hydroxyalkyl)(Cy),
—(C₂-C₆-alkenyl)(Het), —(C₂-C₆-alkenyl)(Cy),
—(NR)(C₁-C₆-alkyl)(Het), —(NR)(C₁-C₆-alkyl)(Cy),
—(NR)C(O)(Het), —(NR)C(O)(Cy),
—(C₃-C₈-cycloalkyl)N(R)C(O)—Cy,
—C₁-C₆-alkyl(NR)C(O)N(C₆-C₁₀-aryl)₂, and
—C₁-C₆-alkyl (optionally substituted with NRR′);
Het is a 3- to 20-membered monocyclic, bicyclic, tricyclic, or tetracyclic heterocyclic ring system wherein 1 to 6 ring members are independently selected from N, O, and S, and wherein the ring system is fully saturated, partially saturated, aromatic, or a combination thereof;
Cy is a 4- to 15-membered monocyclic, bicyclic, or tricyclic carbocyclic ring system that is fully saturated, partially saturated, aromatic, or a combination thereof;
Het and Cy are optionally substituted with 1 to 6 substituents independently selected from the group consisting of halo, —OH, —CN, oxo, thio, —NRR′, —C₁-C₆-alkyl, —C₁-C₆-haloalkyl, —C₃-C₈-cycloalkyl, —(C₁-C₆-alkyl)(NH)₀₋₁(C₃-C₈-cycloalkyl), —C₆-C₁₀-aryl, —O—C₆-C₁₀-aryl, —C(O)—C₆-C₁₀-aryl, —C(O)NH—C₆-C₁₀-aryl, —S(O)₀₋₂—C₆-C₁₀-aryl, —(C₁-C₆-alkyl)-(O)₀₋₁—(C₆-C₁₀-aryl), —C(O)NRR′, —C(O)OR, 5- to 10-membered heteroaryl (wherein 1-4 heteroaryl members are independently selected from N, O, and S), —C(O)(5- to 10-membered heteroaryl (wherein 1-4 heteroaryl members are independently selected from N, O, and S)), —NR(C₁-C₆-alkyl)(5- to 10-membered heteroaryl (wherein 1-4 heteroaryl members are independently selected from N, O, and S), 5- to 8-membered heterocycloalkyl (wherein 1-4 ring members are independently selected from N, O, and S) optionally fused to C₆-C₁₀-aryl, —(C₁-C₆-alkyl)(CONH)₀₋₁(5- to 8-membered heterocycloalkyl (wherein 1-4 ring members are independently selected from N, O, and S));
wherein any alkyl, cycloalkyl, heterocycloalkyl, aryl, or heteroaryl moiety is substituted by 1 to 3 substituents selected from the group consisting of halo, —CN, oxo, —OH, —NRR′, —C₁-C₆-alkyl, —C₁-C₆-haloalkyl, and —(C₁-C₆-alkyl)(C₆-C₁₀-aryl);
each instance of R and R′ is independently selected from H and C₁-C₆-alkyl;
or R and R′, together with the nitrogen atom to which they are bound, form a 5- to 8-membered heterocycloalkyl (wherein 1-3 additional ring members are independently selected from N, O, and S);
n is 0 or 1;
and wherein the compound is not:

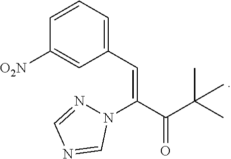 [SMILES: CC(C)(C)C(=O)/C(=C/c1cccc([N+](=O)[O-])c1)n1cncn1]

2. The compound or pharmaceutically acceptable salt thereof according to claim 1, wherein R¹ is —NO₂.

3. The compound or pharmaceutically acceptable salt thereof according to claim 1 or 2, wherein n is 0.

4. The compound or pharmaceutically acceptable salt thereof according to any of claims 1 to 3, wherein X is —NR^{X}(R²).

5. The compound or pharmaceutically acceptable salt thereof according to any of claims 1 to 4, wherein R^{X} is H.

6. The compound or pharmaceutically acceptable salt thereof according to any of claims 1 to 5, wherein R² is selected from the group consisting of —(NR)(C₁-C₆-alkyl)(Het), —(NR)(C₁-C₆-alkyl)(Cy), Cy, and —C₁-C₆-alkyl (optionally substituted with NRR′).

7. The compound or pharmaceutically acceptable salt thereof according to any of claims 1 to 6, wherein Cy is a 6- to 12-membered monocyclic or bicyclic carbocyclic ring system.

8. The compound or pharmaceutically acceptable salt thereof according to any of claims 1 to 7, wherein Cy is —C₆-C₁₀-aryl.

9. The compound or pharmaceutically acceptable salt thereof according to any of claims 1 to 6, wherein Het is a 5- to 12-membered monocyclic or bicyclic heterocyclic ring system.

10. A compound or pharmaceutically acceptable salt thereof selected from the following table:

| | |
|---|---|
| 1 | 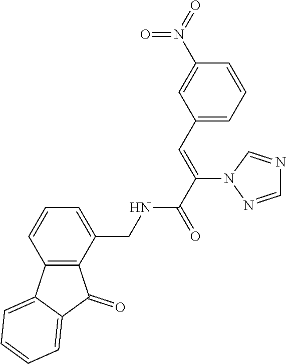 [SMILES: O=C(NCc1cccc2c1C(=O)c1ccccc1-2)/C(=C/c1cccc([N+](=O)[O-])c1)n1cncn1] |
| 2 | 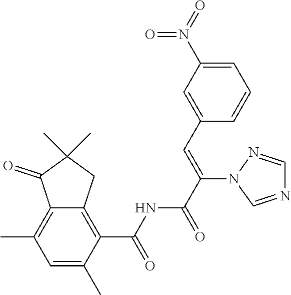 [SMILES: Cc1cc(C)c2c(c1C(=O)NC(=O)/C(=C/c1cccc([N+](=O)[O-])c1)n1cncn1)CC(C)(C)C2=O] |
| 3 | 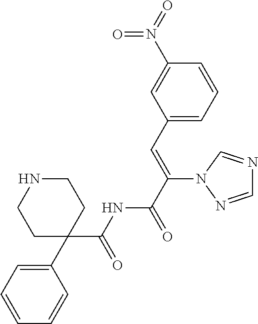 [SMILES: O=C(NC(=O)C1(c2ccccc2)CCNCC1)/C(=C/c1cccc([N+](=O)[O-])c1)n1cncn1] |
| 4 | 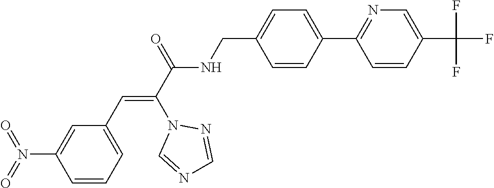 [SMILES: O=C(NCc1ccc(-c2ccc(C(F)(F)F)cn2)cc1)/C(=C/c1cccc([N+](=O)[O-])c1)n1cncn1] |
| 5 | 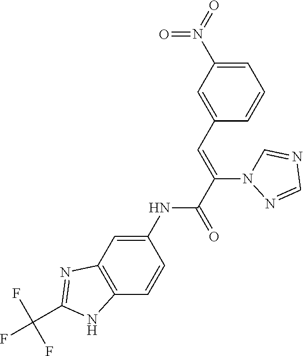 [SMILES: O=C(Nc1ccc2[nH]c(C(F)(F)F)nc2c1)/C(=C/c1cccc([N+](=O)[O-])c1)n1cncn1] |
| 6 | 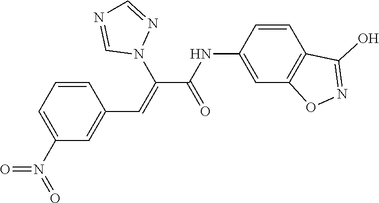 [SMILES: O=C(Nc1ccc2c(O)noc2c1)/C(=C/c1cccc([N+](=O)[O-])c1)n1cncn1] |
| 7 | 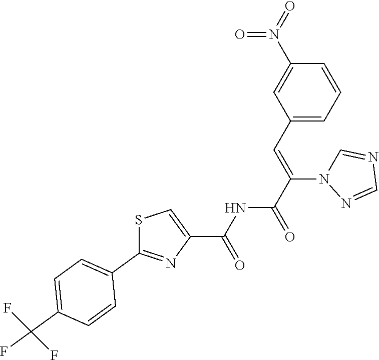 [SMILES: O=C(NC(=O)c1csc(-c2ccc(C(F)(F)F)cc2)n1)/C(=C/c1cccc([N+](=O)[O-])c1)n1cncn1] |
| 8 | 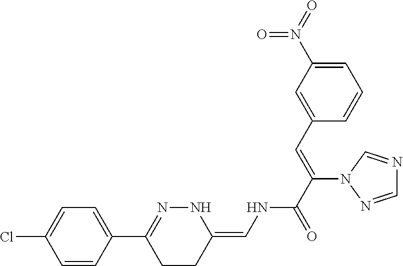 [SMILES: O=C(N/C=C1/CCC(c2ccc(Cl)cc2)=NN1)/C(=C/c1cccc([N+](=O)[O-])c1)n1cncn1] |
| 9 | 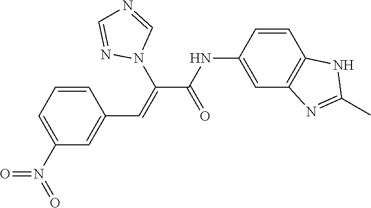 [SMILES: Cc1nc2cc(NC(=O)/C(=C/c3cccc([N+](=O)[O-])c3)n3cncn3)ccc2[nH]1] |
| 10 | 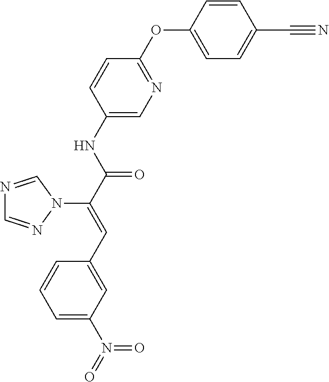 [SMILES: N#Cc1ccc(Oc2ccc(NC(=O)/C(=C/c3cccc([N+](=O)[O-])c3)n3cncn3)cn2)cc1] |
| 11 | 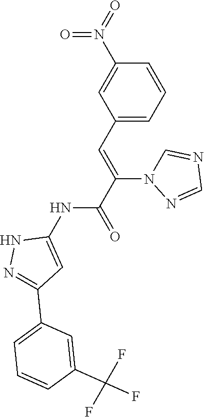 [SMILES: O=C(Nc1cc(-c2cccc(C(F)(F)F)c2)n[nH]1)/C(=C/c1cccc([N+](=O)[O-])c1)n1cncn1] |
| 12 | 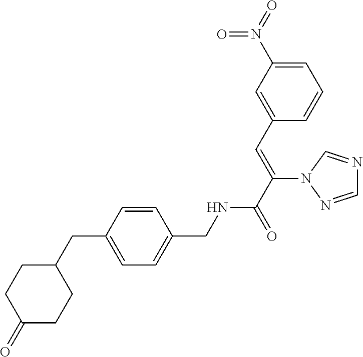 [SMILES: O=C1CCC(Cc2ccc(CNC(=O)/C(=C/c3cccc([N+](=O)[O-])c3)n3cncn3)cc2)CC1] |
| 13 | 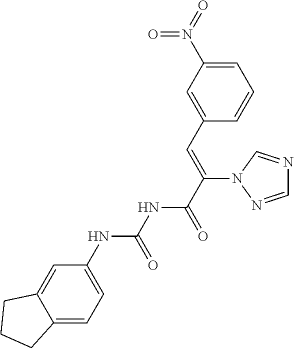 [SMILES: O=C(NC(=O)/C(=C/c1cccc([N+](=O)[O-])c1)n1cncn1)Nc1ccc2c(c1)CCC2] |
| 14 | 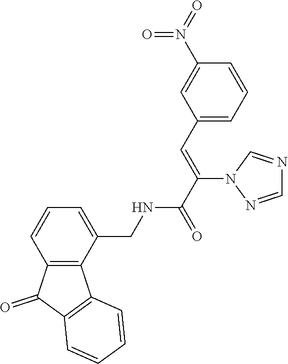 [SMILES: O=C(NCc1cccc2c1-c1ccccc1C2=O)/C(=C/c1cccc([N+](=O)[O-])c1)n1cncn1] |
| 15 | 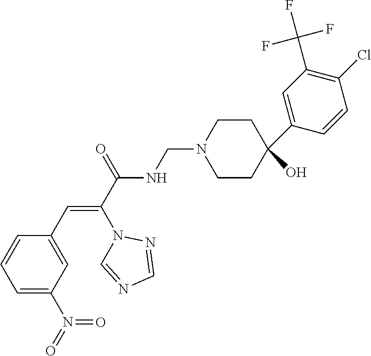 |
| 16 | 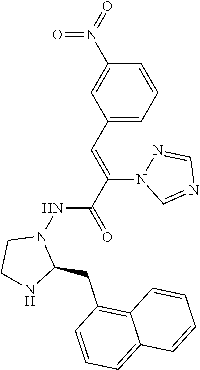 [SMILES: O=C(NN1CCN[C@@H]1Cc1cccc2ccccc12)/C(=C/c1cccc([N+](=O)[O-])c1)n1cncn1] |
| 17 | 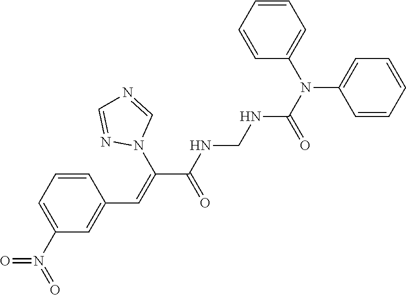 [SMILES: O=C(NCNC(=O)N(c1ccccc1)c1ccccc1)/C(=C/c1cccc([N+](=O)[O-])c1)n1cncn1] |
| 18 | 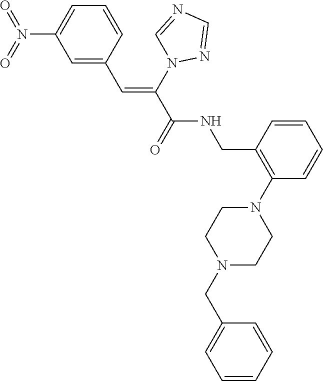 [SMILES: O=C(NCc1ccccc1N1CCN(Cc2ccccc2)CC1)/C(=C/c1cccc([N+](=O)[O-])c1)n1cncn1] |
| 19 | 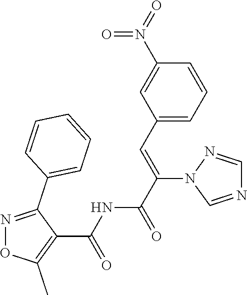 [SMILES: Cc1onc(-c2ccccc2)c1C(=O)NC(=O)/C(=C/c1cccc([N+](=O)[O-])c1)n1cncn1] |
| 20 | 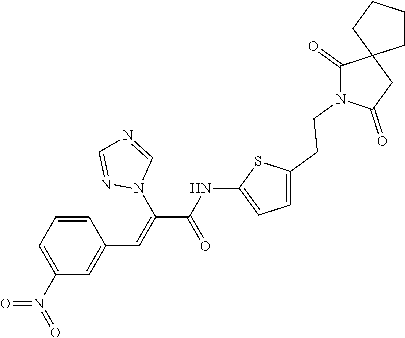 [SMILES: O=C(Nc1ccc(CCN2C(=O)CC3(CCCC3)C2=O)s1)/C(=C/c1cccc([N+](=O)[O-])c1)n1cncn1] |
| 21 | 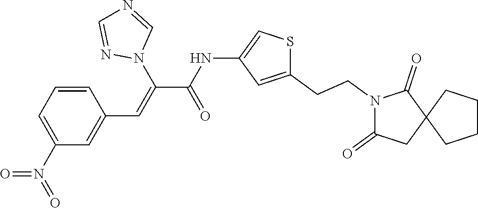 [SMILES: O=C(Nc1csc(CCN2C(=O)CC3(CCCC3)C2=O)c1)/C(=C/c1cccc([N+](=O)[O-])c1)n1cncn1] |
| 22 | 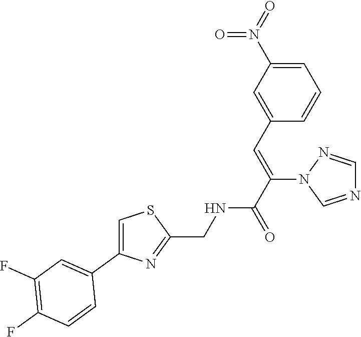 [SMILES: O=C(NCc1nc(-c2ccc(F)c(F)c2)cs1)/C(=C/c1cccc([N+](=O)[O-])c1)n1cncn1] |
| 23 | 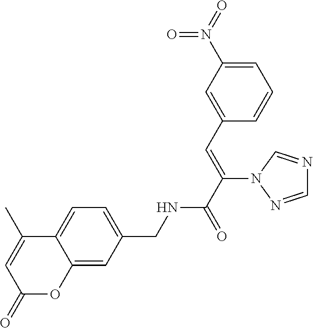 [SMILES: Cc1cc(=O)oc2cc(CNC(=O)/C(=C/c3cccc([N+](=O)[O-])c3)n3cncn3)ccc12] |
| 24 | 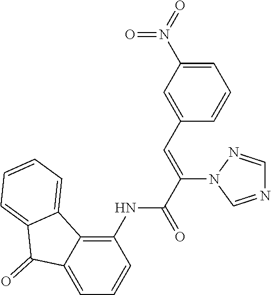 [SMILES: O=C(Nc1cccc2c1-c1ccccc1C2=O)/C(=C/c1cccc([N+](=O)[O-])c1)n1cncn1] |
| 25 | 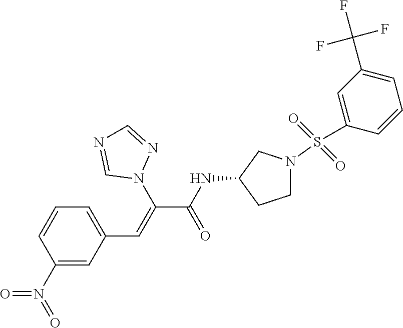 [SMILES: O=C(N[C@H]1CCN(S(=O)(=O)c2cccc(C(F)(F)F)c2)C1)/C(=C/c1cccc([N+](=O)[O-])c1)n1cncn1] |
| 26 | 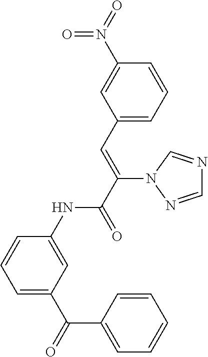 [SMILES: O=C(Nc1cccc(C(=O)c2ccccc2)c1)/C(=C/c1cccc([N+](=O)[O-])c1)n1cncn1] |
| 27 | 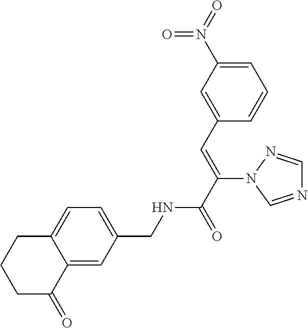 [SMILES: O=C(NCc1ccc2c(c1)C(=O)CCC2)/C(=C/c1cccc([N+](=O)[O-])c1)n1cncn1] |
| 28 | 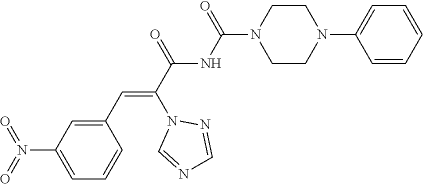 [SMILES: O=C(NC(=O)N1CCN(c2ccccc2)CC1)/C(=C/c1cccc([N+](=O)[O-])c1)n1cncn1] |
| 29 | 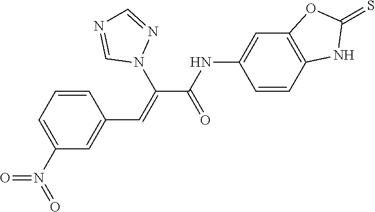 [SMILES: O=C(Nc1ccc2[nH]c(=S)oc2c1)/C(=C/c1cccc([N+](=O)[O-])c1)n1cncn1] |
| 30 | 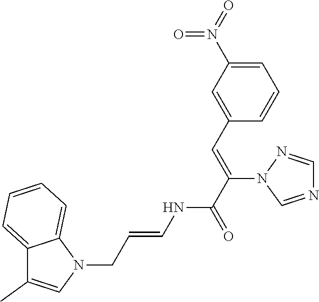 [SMILES: Cc1cn(C/C=C/NC(=O)/C(=C/c2cccc([N+](=O)[O-])c2)n2cncn2)c2ccccc12] |
| 31 | 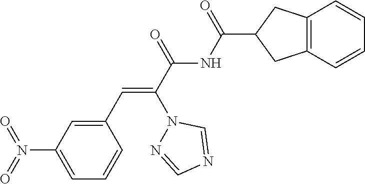 [SMILES: O=C(NC(=O)C1Cc2ccccc2C1)/C(=C/c1cccc([N+](=O)[O-])c1)n1cncn1] |
| 32 | 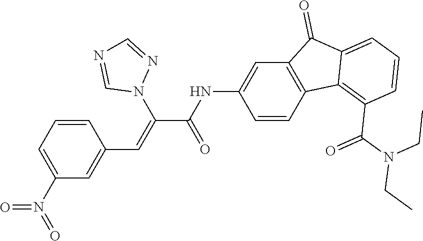 [SMILES: CCN(CC)C(=O)c1cccc2c1-c1ccc(NC(=O)/C(=C/c3cccc([N+](=O)[O-])c3)n3cncn3)cc1C2=O] |
| 33 | 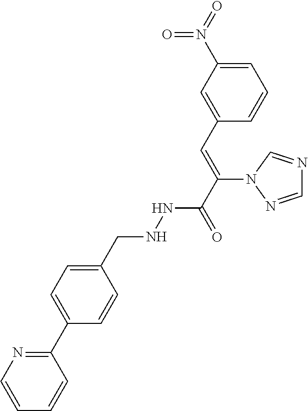 [SMILES: O=C(NNCc1ccc(-c2ccccn2)cc1)/C(=C/c1cccc([N+](=O)[O-])c1)n1cncn1] |
| 34 | 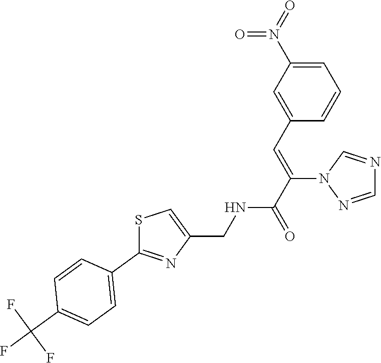 [SMILES: O=C(NCc1csc(-c2ccc(C(F)(F)F)cc2)n1)/C(=C/c1cccc([N+](=O)[O-])c1)n1cncn1] |
| 35 | 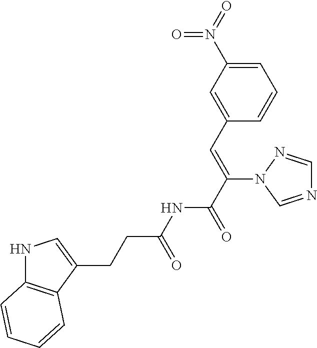 [SMILES: O=C(CCc1c[nH]c2ccccc12)NC(=O)/C(=C/c1cccc([N+](=O)[O-])c1)n1cncn1] |
| 36 | 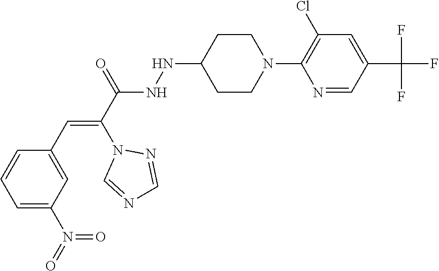 [SMILES: O=C(NNC1CCN(c2ncc(C(F)(F)F)cc2Cl)CC1)/C(=C/c1cccc([N+](=O)[O-])c1)n1cncn1] |
| 37 | 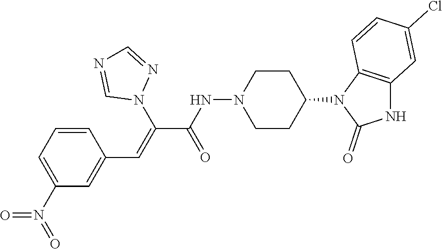 |
| 38 | 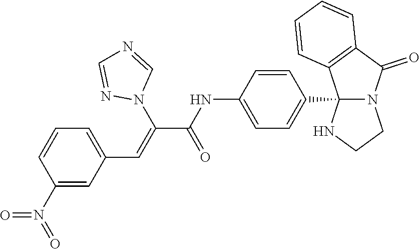 [SMILES: O=C(Nc1ccc([C@@]23NCCN2C(=O)c2ccccc23)cc1)/C(=C/c1cccc([N+](=O)[O-])c1)n1cncn1] |
| 39 | 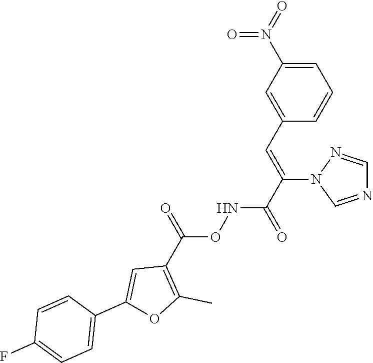 [SMILES: Cc1oc(-c2ccc(F)cc2)cc1C(=O)ONC(=O)/C(=C/c1cccc([N+](=O)[O-])c1)n1cncn1] |
| 40 | 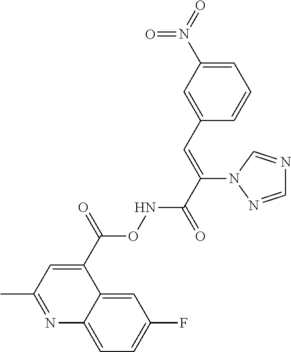 [SMILES: Cc1cc(C(=O)ONC(=O)/C(=C/c2cccc([N+](=O)[O-])c2)n2cncn2)c2cc(F)ccc2n1] |
| 41 | 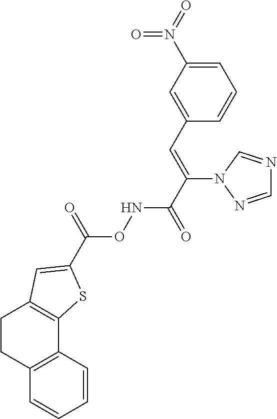 [SMILES: O=C(NOC(=O)c1cc2c(s1)-c1ccccc1CC2)/C(=C/c1cccc([N+](=O)[O-])c1)n1cncn1] |
| 42 | 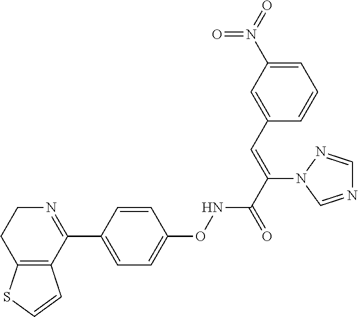 [SMILES: O=C(NOc1ccc(C2=NCCc3sccc32)cc1)/C(=C/c1cccc([N+](=O)[O-])c1)n1cncn1] |
| 43 | 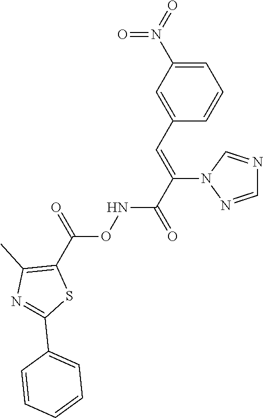 [SMILES: Cc1nc(-c2ccccc2)sc1C(=O)ONC(=O)/C(=C/c1cccc([N+](=O)[O-])c1)n1cncn1] |
| 44 | 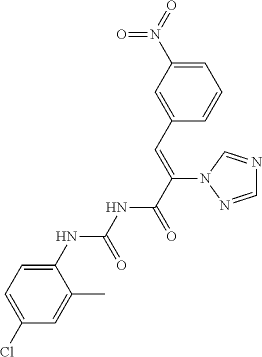 [SMILES: Cc1cc(Cl)ccc1NC(=O)NC(=O)/C(=C/c1cccc([N+](=O)[O-])c1)n1cncn1] |
| 45 | 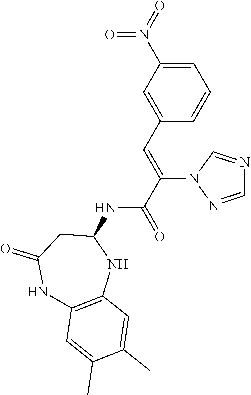 [SMILES: Cc1cc2c(cc1C)N[C@H](NC(=O)/C(=C/c1cccc([N+](=O)[O-])c1)n1cncn1)CC(=O)N2] |
| 46 | 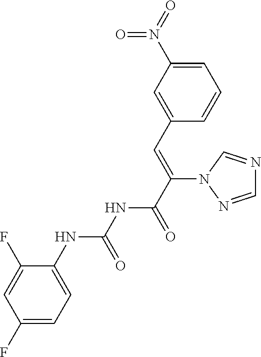 [SMILES: O=C(NC(=O)/C(=C/c1cccc([N+](=O)[O-])c1)n1cncn1)Nc1ccc(F)cc1F] |
| 47 | 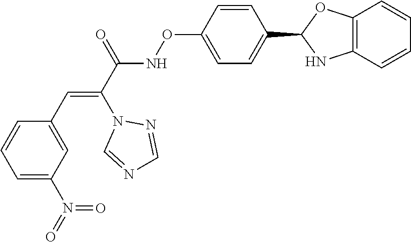 [SMILES: O=C(NOc1ccc([C@@H]2Nc3ccccc3O2)cc1)/C(=C/c1cccc([N+](=O)[O-])c1)n1cncn1] |
| 48 | 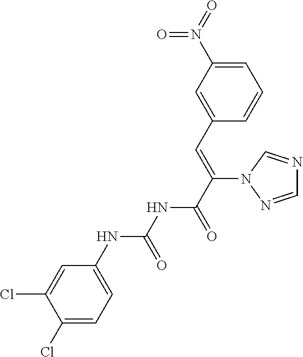 [SMILES: O=C(NC(=O)/C(=C/c1cccc([N+](=O)[O-])c1)n1cncn1)Nc1ccc(Cl)c(Cl)c1] |
| 49 | 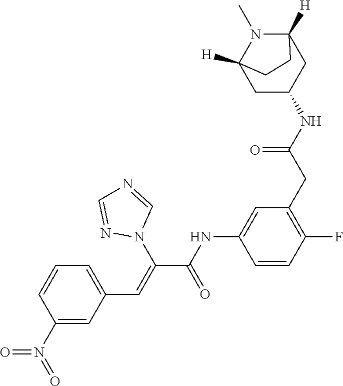 [SMILES: CN1[C@@H]2CC[C@H]1C[C@H](NC(=O)Cc1cc(NC(=O)/C(=C/c3cccc([N+](=O)[O-])c3)n3cncn3)ccc1F)C2] |
| 50 | 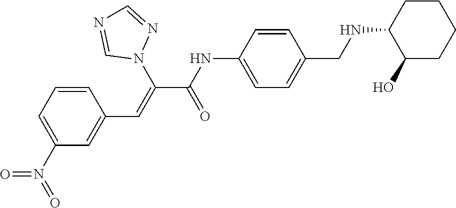 [SMILES: O=C(Nc1ccc(CN[C@@H]2CCCC[C@H]2O)cc1)/C(=C/c1cccc([N+](=O)[O-])c1)n1cncn1] |
| 51 | 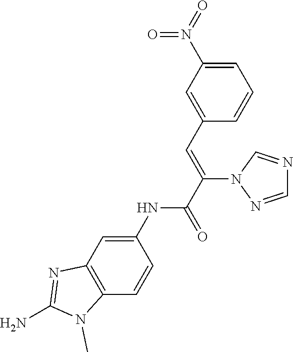 [SMILES: Cn1c(N)nc2cc(NC(=O)/C(=C/c3cccc([N+](=O)[O-])c3)n3cncn3)ccc21] |
| 52 | 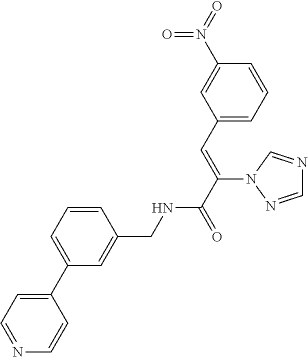 [SMILES: O=C(NCc1cccc(-c2ccncc2)c1)/C(=C/c1cccc([N+](=O)[O-])c1)n1cncn1] |
| 53 | 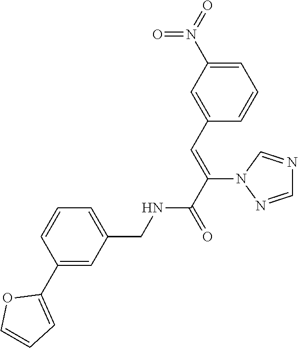 [SMILES: O=C(NCc1cccc(-c2ccco2)c1)/C(=C/c1cccc([N+](=O)[O-])c1)n1cncn1] |
| 54 | 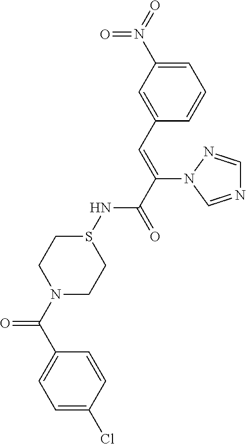 |
| 55 | 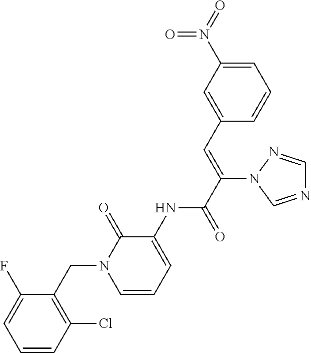 [SMILES: O=C(Nc1cccn(Cc2c(F)cccc2Cl)c1=O)/C(=C/c1cccc([N+](=O)[O-])c1)n1cncn1] |
| 56 | 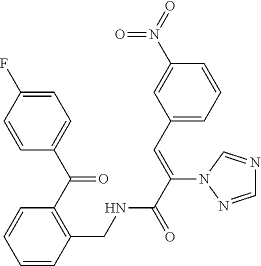 [SMILES: O=C(NCc1ccccc1C(=O)c1ccc(F)cc1)/C(=C/c1cccc([N+](=O)[O-])c1)n1cncn1] |
| 57 | 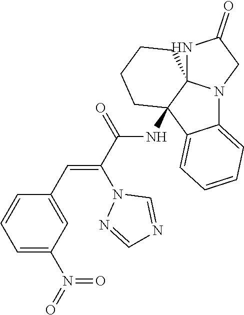 [SMILES: O=C1CN2c3ccccc3[C@]3(NC(=O)/C(=C/c4cccc([N+](=O)[O-])c4)n4cncn4)CCCC[C@]23N1] |
| 58 | 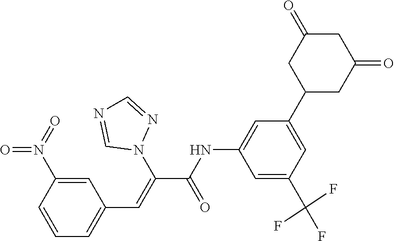 [SMILES: O=C1CC(=O)CC(c2cc(NC(=O)/C(=C/c3cccc([N+](=O)[O-])c3)n3cncn3)cc(C(F)(F)F)c2)C1] |
| 59 | 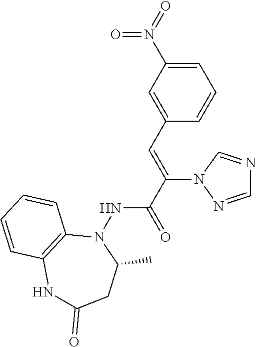 [SMILES: C[C@@H]1CC(=O)Nc2ccccc2N1NC(=O)/C(=C/c1cccc([N+](=O)[O-])c1)n1cncn1] |
| 60 | 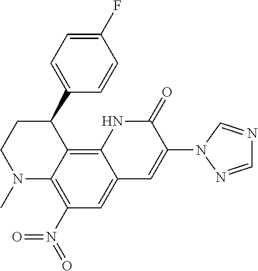 [SMILES: CN1CC[C@@H](c2ccc(F)cc2)c2c1c([N+](=O)[O-])cc1cc(-n3cncn3)c(=O)[nH]c21] |
| 61 | 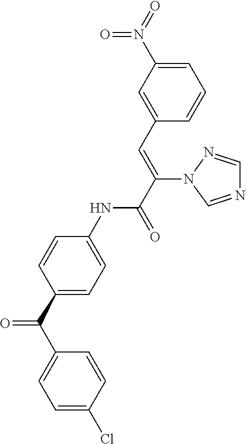 |
| 62 | 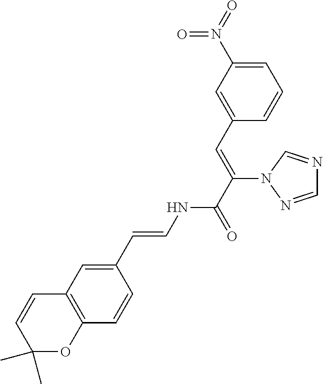 [SMILES: CC1(C)C=Cc2cc(/C=C/NC(=O)/C(=C/c3cccc([N+](=O)[O-])c3)n3cncn3)ccc2O1] |
| 63 | 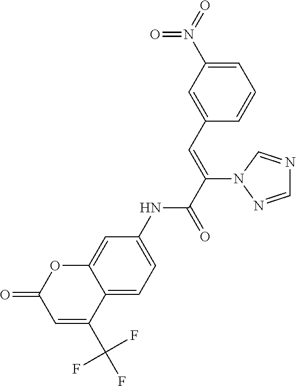 [SMILES: O=C(Nc1ccc2c(C(F)(F)F)cc(=O)oc2c1)/C(=C/c1cccc([N+](=O)[O-])c1)n1cncn1] |
| 64 | 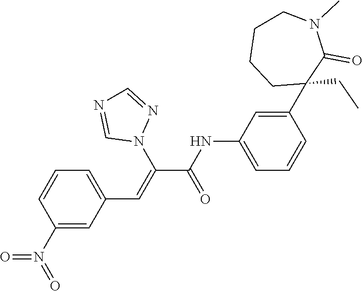 [SMILES: CC[C@]1(c2cccc(NC(=O)/C(=C/c3cccc([N+](=O)[O-])c3)n3cncn3)c2)CCCCN(C)C1=O] |
| 65 | 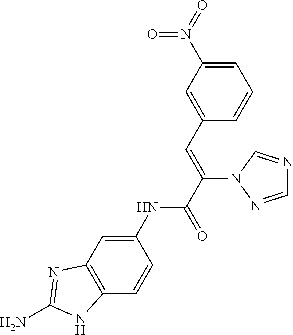 [SMILES: Nc1nc2cc(NC(=O)/C(=C/c3cccc([N+](=O)[O-])c3)n3cncn3)ccc2[nH]1] |
| 66 | 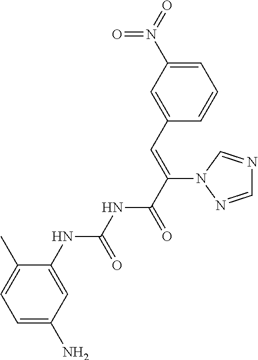 [SMILES: Cc1ccc(N)cc1NC(=O)NC(=O)/C(=C/c1cccc([N+](=O)[O-])c1)n1cncn1] |
| 67 | 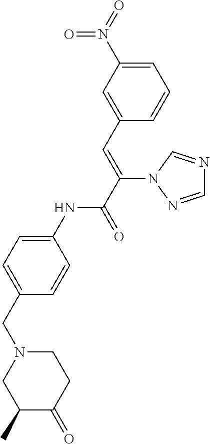 [SMILES: C[C@H]1CN(Cc2ccc(NC(=O)/C(=C/c3cccc([N+](=O)[O-])c3)n3cncn3)cc2)CCC1=O] |
| 68 | 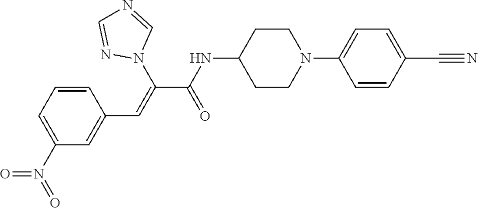 [SMILES: N#Cc1ccc(N2CCC(NC(=O)/C(=C/c3cccc([N+](=O)[O-])c3)n3cncn3)CC2)cc1] |
| 69 | 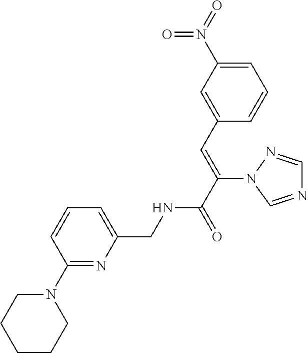 [SMILES: O=C(NCc1cccc(N2CCCCC2)n1)/C(=C/c1cccc([N+](=O)[O-])c1)n1cncn1] |
| 70 | 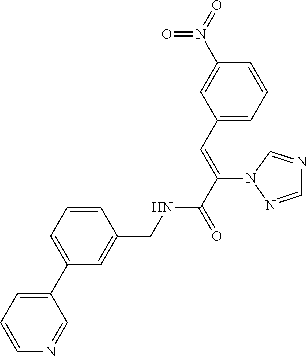 [SMILES: O=C(NCc1cccc(-c2cccnc2)c1)/C(=C/c1cccc([N+](=O)[O-])c1)n1cncn1] |
| 71 | 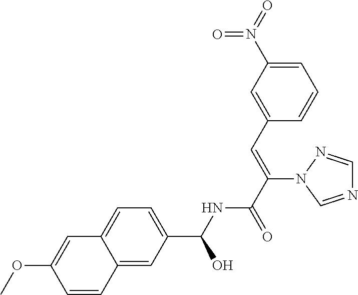 [SMILES: COc1ccc2cc([C@H](O)NC(=O)/C(=C/c3cccc([N+](=O)[O-])c3)n3cncn3)ccc2c1] |
| 72 | 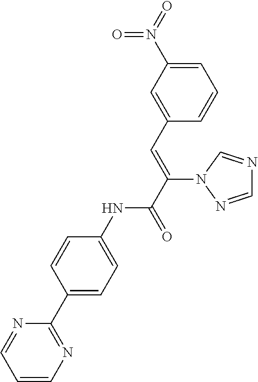 [SMILES: O=C(Nc1ccc(-c2ncccn2)cc1)/C(=C/c1cccc([N+](=O)[O-])c1)n1cncn1] |
| 73 | 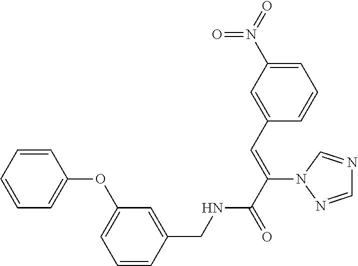 [SMILES: O=C(NCc1cccc(Oc2ccccc2)c1)/C(=C/c1cccc([N+](=O)[O-])c1)n1cncn1] |
| 74 | 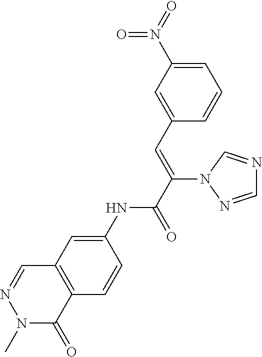 [SMILES: Cn1ncc2cc(NC(=O)/C(=C/c3cccc([N+](=O)[O-])c3)n3cncn3)ccc2c1=O] |
| 75 | 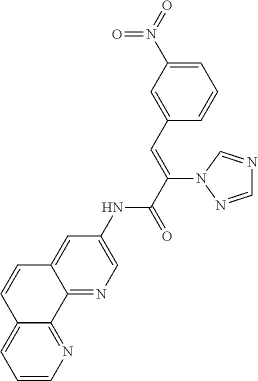 [SMILES: O=C(Nc1cnc2c(ccc3cccnc32)c1)/C(=C/c1cccc([N+](=O)[O-])c1)n1cncn1] |
| 76 | 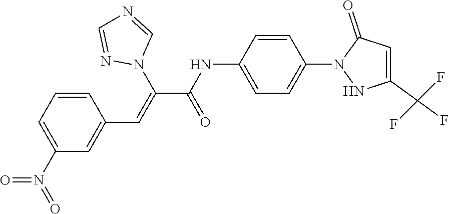 [SMILES: O=C(Nc1ccc(-n2[nH]c(C(F)(F)F)cc2=O)cc1)/C(=C/c1cccc([N+](=O)[O-])c1)n1cncn1] |
| 77 | 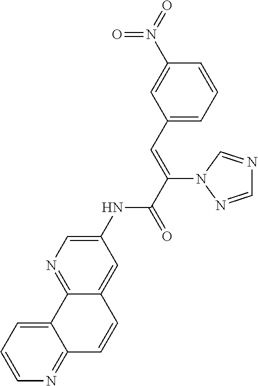 [SMILES: O=C(Nc1cnc2c(ccc3ncccc32)c1)/C(=C/c1cccc([N+](=O)[O-])c1)n1cncn1] |
| 78 | 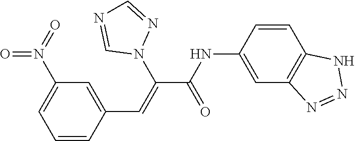 [SMILES: O=C(Nc1ccc2[nH]nnc2c1)/C(=C/c1cccc([N+](=O)[O-])c1)n1cncn1] |
| 79 | 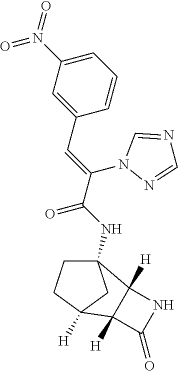 [SMILES: O=C(N[C@@]12CC[C@@H](C1)[C@H]1C(=O)N[C@H]12)/C(=C/c1cccc([N+](=O)[O-])c1)n1cncn1] |
| 80 | 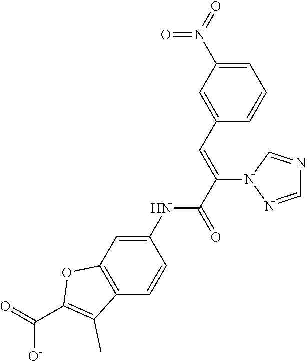 [SMILES: Cc1c(C(=O)[O-])oc2cc(NC(=O)/C(=C/c3cccc([N+](=O)[O-])c3)n3cncn3)ccc12] |
| 81 | 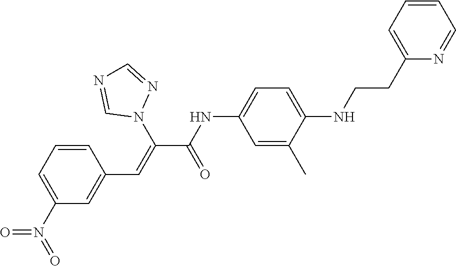 [SMILES: Cc1cc(NC(=O)/C(=C/c2cccc([N+](=O)[O-])c2)n2cncn2)ccc1NCCc1ccccn1] |
| 82 | 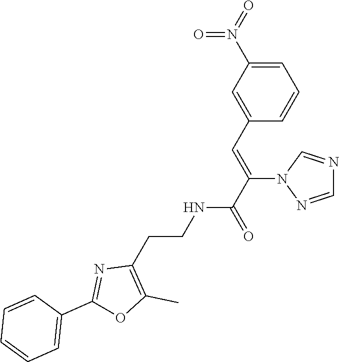 [SMILES: Cc1oc(-c2ccccc2)nc1CCNC(=O)/C(=C/c1cccc([N+](=O)[O-])c1)n1cncn1] |
| 83 | 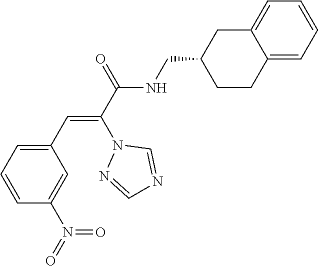 [SMILES: O=C(NC[C@H]1CCc2ccccc2C1)/C(=C/c1cccc([N+](=O)[O-])c1)n1cncn1] |
| 84 | 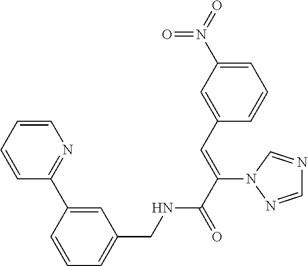 [SMILES: O=C(NCc1cccc(-c2ccccn2)c1)/C(=C/c1cccc([N+](=O)[O-])c1)n1cncn1] |
| 85 | 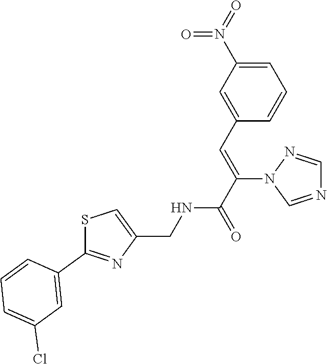 [SMILES: O=C(NCc1csc(-c2cccc(Cl)c2)n1)/C(=C/c1cccc([N+](=O)[O-])c1)n1cncn1] |
| 86 | 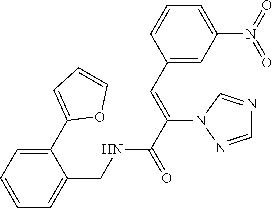 [SMILES: O=C(NCc1ccccc1-c1ccco1)/C(=C/c1cccc([N+](=O)[O-])c1)n1cncn1] |
| 87 | 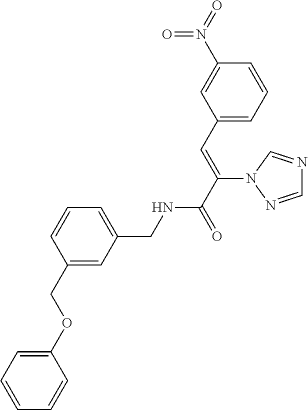 [SMILES: O=C(NCc1cccc(COc2ccccc2)c1)/C(=C/c1cccc([N+](=O)[O-])c1)n1cncn1] |
| 88 | 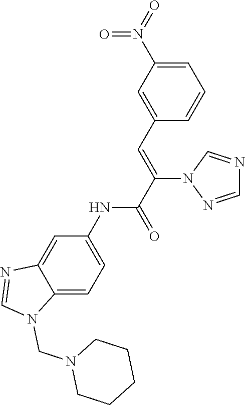 [SMILES: O=C(Nc1ccc2c(c1)ncn2CN1CCCCC1)/C(=C/c1cccc([N+](=O)[O-])c1)n1cncn1] |
| 89 | 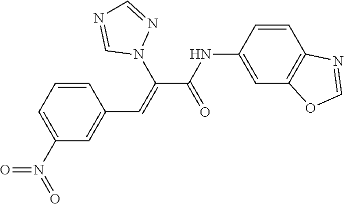 [SMILES: O=C(Nc1ccc2ncoc2c1)/C(=C/c1cccc([N+](=O)[O-])c1)n1cncn1] |
| 90 | 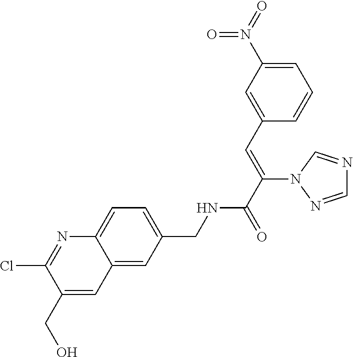 [SMILES: O=C(NCc1ccc2nc(Cl)c(CO)cc2c1)/C(=C/c1cccc([N+](=O)[O-])c1)n1cncn1] |
| 91 | 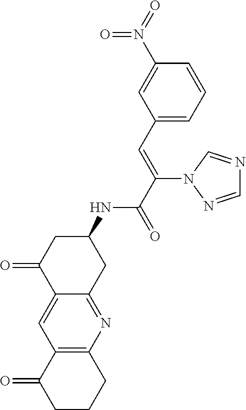 [SMILES: O=C(N[C@@H]1CC(=O)c2cc3c(nc2C1)CCCC3=O)/C(=C/c1cccc([N+](=O)[O-])c1)n1cncn1] |
| 92 | 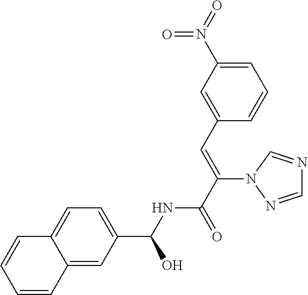 [SMILES: O=C(N[C@@H](O)c1ccc2ccccc2c1)/C(=C/c1cccc([N+](=O)[O-])c1)n1cncn1] |
| 93 | 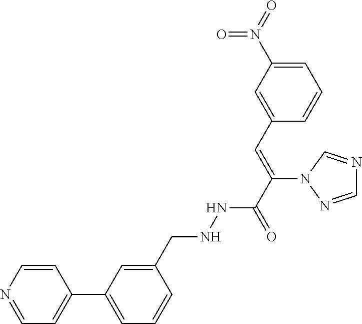 [SMILES: O=C(NNCc1cccc(-c2ccncc2)c1)/C(=C/c1cccc([N+](=O)[O-])c1)n1cncn1] |
| 94 | 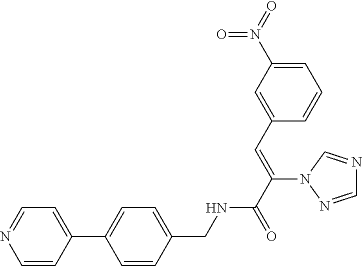 [SMILES: O=C(NCc1ccc(-c2ccncc2)cc1)/C(=C/c1cccc([N+](=O)[O-])c1)n1cncn1] |
| 95 | 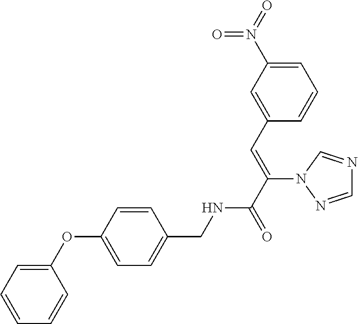 [SMILES: O=C(NCc1ccc(Oc2ccccc2)cc1)/C(=C/c1cccc([N+](=O)[O-])c1)n1cncn1] |
| 96 | 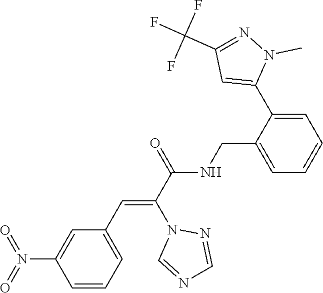 [SMILES: Cn1nc(C(F)(F)F)cc1-c1ccccc1CNC(=O)/C(=C/c1cccc([N+](=O)[O-])c1)n1cncn1] |
| 97 | 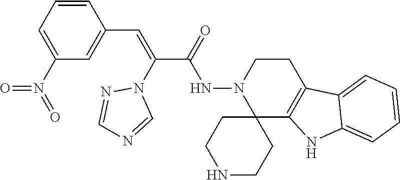 [SMILES: O=C(NN1CCc2c([nH]c3ccccc23)C12CCNCC2)/C(=C/c1cccc([N+](=O)[O-])c1)n1cncn1] |
| 98 | 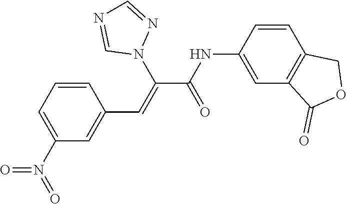 [SMILES: O=C(Nc1ccc2c(c1)C(=O)OC2)/C(=C/c1cccc([N+](=O)[O-])c1)n1cncn1] |
| 99 | 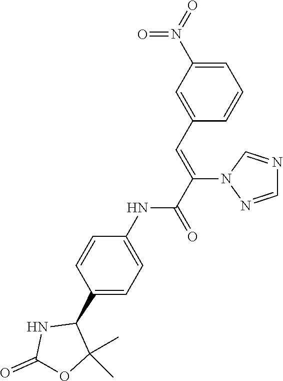 [SMILES: CC1(C)OC(=O)N[C@H]1c1ccc(NC(=O)/C(=C/c2cccc([N+](=O)[O-])c2)n2cncn2)cc1] |
| 100 | 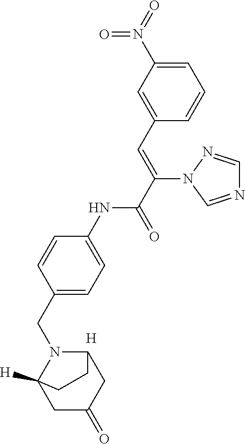 |
| 101 | 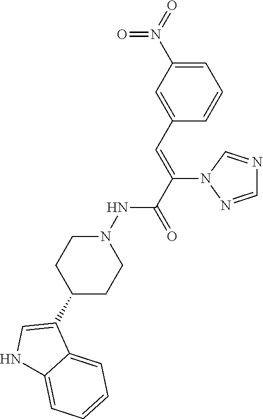 |
| 102 | 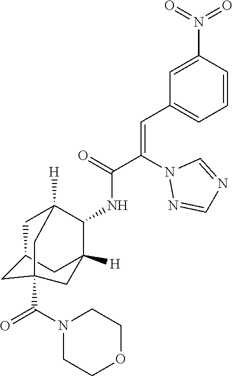 [SMILES: O=C(N[C@H]1[C@@H]2C[C@@H]3C[C@H]1C[C@@](C(=O)N1CCOCC1)(C3)C2)/C(=C/c1cccc([N+](=O)[O-])c1)n1cncn1] |
| 103 | 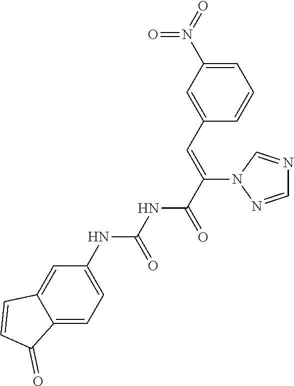 [SMILES: O=C(NC(=O)/C(=C/c1cccc([N+](=O)[O-])c1)n1cncn1)Nc1ccc2c(c1)C=CC2=O] |
| 104 | 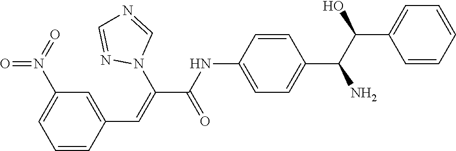 [SMILES: N[C@@H](c1ccc(NC(=O)/C(=C/c2cccc([N+](=O)[O-])c2)n2cncn2)cc1)[C@@H](O)c1ccccc1] |
| 105 | 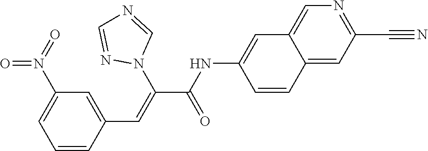 [SMILES: N#Cc1cc2ccc(NC(=O)/C(=C/c3cccc([N+](=O)[O-])c3)n3cncn3)cc2cn1] |
| 106 | 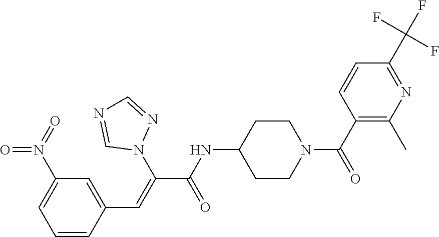 [SMILES: Cc1nc(C(F)(F)F)ccc1C(=O)N1CCC(NC(=O)/C(=C/c2cccc([N+](=O)[O-])c2)n2cncn2)CC1] |
| 107 | 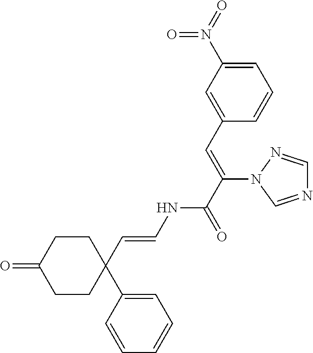 [SMILES: O=C1CCC(/C=C/NC(=O)/C(=C/c2cccc([N+](=O)[O-])c2)n2cncn2)(c2ccccc2)CC1] |
| 108 | 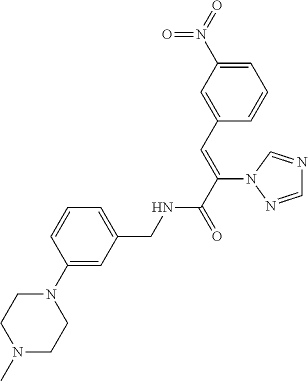 [SMILES: CN1CCN(c2cccc(CNC(=O)/C(=C/c3cccc([N+](=O)[O-])c3)n3cncn3)c2)CC1] |
| 109 | 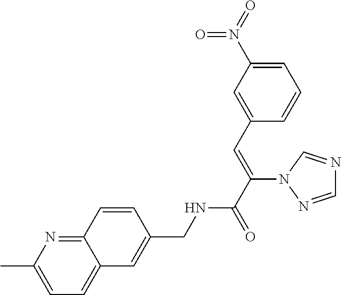 [SMILES: Cc1ccc2cc(CNC(=O)/C(=C/c3cccc([N+](=O)[O-])c3)n3cncn3)ccc2n1] |
| 110 | 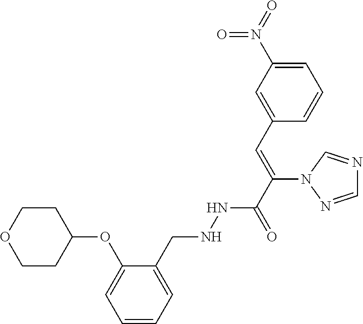 [SMILES: O=C(NNCc1ccccc1OC1CCOCC1)/C(=C/c1cccc([N+](=O)[O-])c1)n1cncn1] |
| 111 | 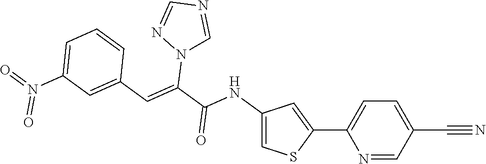 [SMILES: N#Cc1ccc(-c2cc(NC(=O)/C(=C/c3cccc([N+](=O)[O-])c3)n3cncn3)cs2)nc1] |
| 112 | 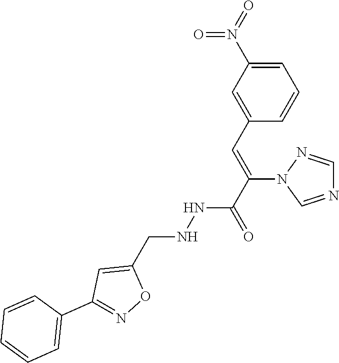 [SMILES: O=C(NNCc1cc(-c2ccccc2)no1)/C(=C/c1cccc([N+](=O)[O-])c1)n1cncn1] |
| 113 | 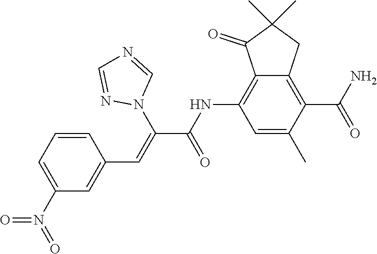 [SMILES: Cc1cc(NC(=O)/C(=C/c2cccc([N+](=O)[O-])c2)n2cncn2)c2c(c1C(N)=O)CC(C)(C)C2=O] |
| 114 | 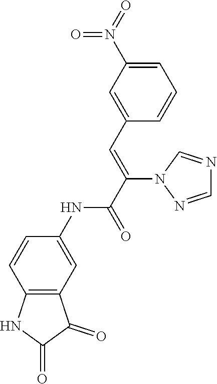 [SMILES: O=C1Nc2ccc(NC(=O)/C(=C/c3cccc([N+](=O)[O-])c3)n3cncn3)cc2C1=O] |
| 115 | 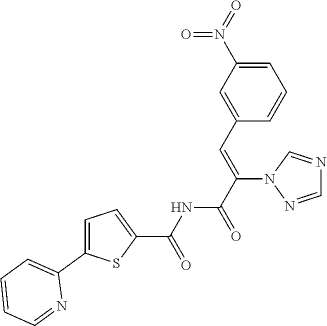 [SMILES: O=C(NC(=O)c1ccc(-c2ccccn2)s1)/C(=C/c1cccc([N+](=O)[O-])c1)n1cncn1] |
| 116 | 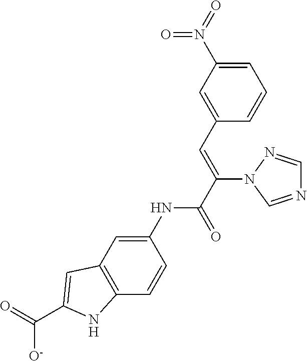 [SMILES: O=C(Nc1ccc2[nH]c(C(=O)[O-])cc2c1)/C(=C/c1cccc([N+](=O)[O-])c1)n1cncn1] |
| 117 | 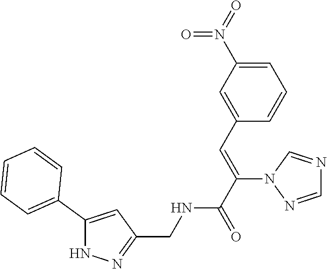 [SMILES: O=C(NCc1cc(-c2ccccc2)[nH]n1)/C(=C/c1cccc([N+](=O)[O-])c1)n1cncn1] |
| 118 | 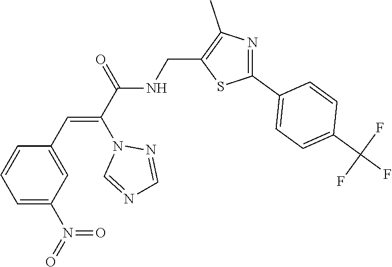 [SMILES: Cc1nc(-c2ccc(C(F)(F)F)cc2)sc1CNC(=O)/C(=C/c1cccc([N+](=O)[O-])c1)n1cncn1] |
| 119 | 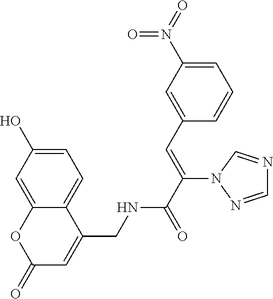 [SMILES: O=C(NCc1cc(=O)oc2cc(O)ccc12)/C(=C/c1cccc([N+](=O)[O-])c1)n1cncn1] |
| 120 | 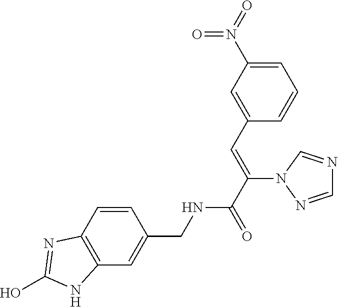 [SMILES: O=C(NCc1ccc2nc(O)[nH]c2c1)/C(=C/c1cccc([N+](=O)[O-])c1)n1cncn1] |
| 121 | 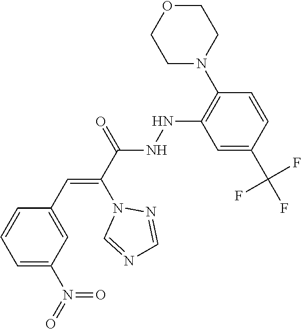 [SMILES: O=C(NNc1cc(C(F)(F)F)ccc1N1CCOCC1)/C(=C/c1cccc([N+](=O)[O-])c1)n1cncn1] |
| 122 | 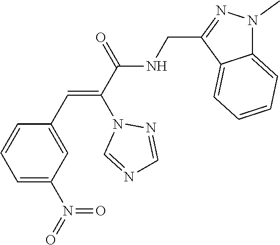 [SMILES: Cn1nc(CNC(=O)/C(=C/c2cccc([N+](=O)[O-])c2)n2cncn2)c2ccccc21] |
| 123 | 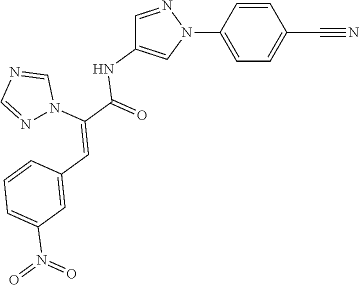 [SMILES: N#Cc1ccc(-n2cc(NC(=O)/C(=C/c3cccc([N+](=O)[O-])c3)n3cncn3)cn2)cc1] |
| 124 | 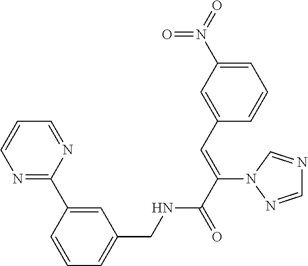 [SMILES: O=C(NCc1cccc(-c2ncccn2)c1)/C(=C/c1cccc([N+](=O)[O-])c1)n1cncn1] |
| 125 | 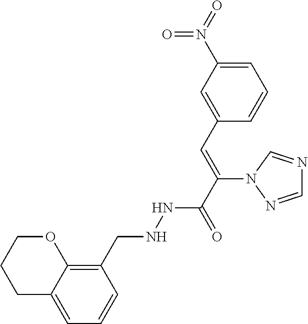 [SMILES: O=C(NNCc1cccc2c1OCCC2)/C(=C/c1cccc([N+](=O)[O-])c1)n1cncn1] |
| 126 | 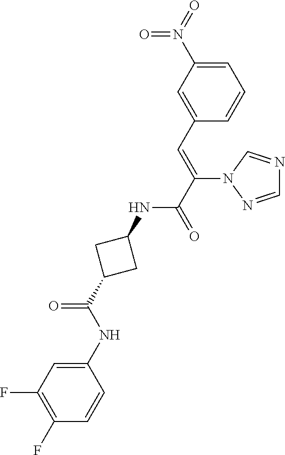 [SMILES: O=C(N[C@H]1C[C@H](C(=O)Nc2ccc(F)c(F)c2)C1)/C(=C/c1cccc([N+](=O)[O-])c1)n1cncn1] |
| 127 | 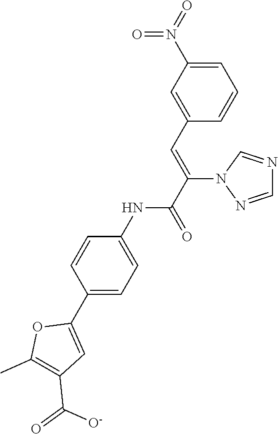 [SMILES: Cc1oc(-c2ccc(NC(=O)/C(=C/c3cccc([N+](=O)[O-])c3)n3cncn3)cc2)cc1C(=O)[O-]] |
| 128 | 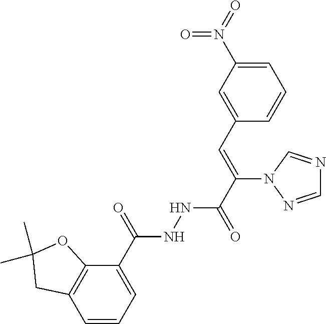 [SMILES: CC1(C)Cc2cccc(C(=O)NNC(=O)/C(=C/c3cccc([N+](=O)[O-])c3)n3cncn3)c2O1] |
| 129 | 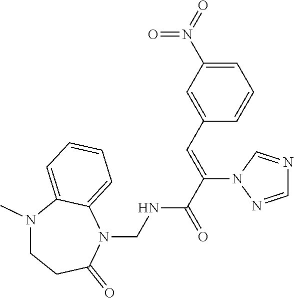 [SMILES: CN1CCC(=O)N(CNC(=O)/C(=C/c2cccc([N+](=O)[O-])c2)n2cncn2)c2ccccc21] |
| 130 | 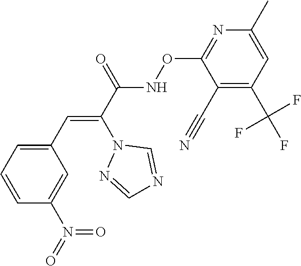 [SMILES: Cc1cc(C(F)(F)F)c(C#N)c(ONC(=O)/C(=C/c2cccc([N+](=O)[O-])c2)n2cncn2)n1] |
| 131 | 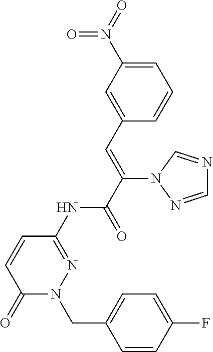 [SMILES: O=C(Nc1ccc(=O)n(Cc2ccc(F)cc2)n1)/C(=C/c1cccc([N+](=O)[O-])c1)n1cncn1] |
| 132 | 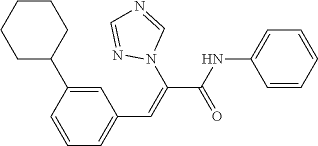 [SMILES: O=C(Nc1ccccc1)/C(=C/c1cccc(C2CCCCC2)c1)n1cncn1] |
| 133 | 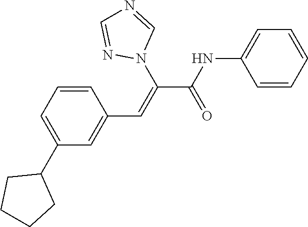 [SMILES: O=C(Nc1ccccc1)/C(=C/c1cccc(C2CCCC2)c1)n1cncn1] |
| 134 | 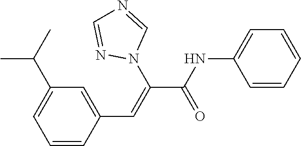 [SMILES: CC(C)c1cccc(/C=C(/C(=O)Nc2ccccc2)n2cncn2)c1] |
| 135 | 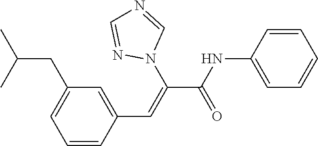 [SMILES: CC(C)Cc1cccc(/C=C(/C(=O)Nc2ccccc2)n2cncn2)c1] |
| 136 | 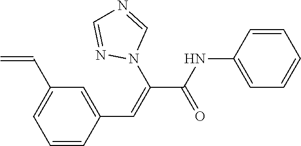 [SMILES: C=Cc1cccc(/C=C(/C(=O)Nc2ccccc2)n2cncn2)c1] |
| 137 | 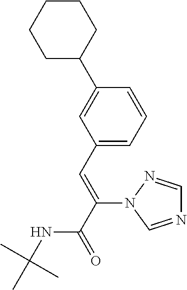 [SMILES: CC(C)(C)NC(=O)/C(=C/c1cccc(C2CCCCC2)c1)n1cncn1] |
| 138 | 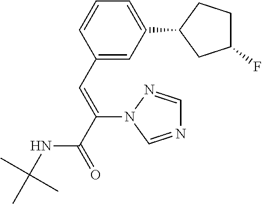 [SMILES: CC(C)(C)NC(=O)/C(=C/c1cccc([C@@H]2CC[C@H](F)C2)c1)n1cncn1] |
| 139 | 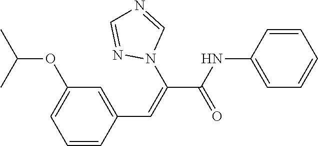 [SMILES: CC(C)Oc1cccc(/C=C(/C(=O)Nc2ccccc2)n2cncn2)c1] |
| 140 | 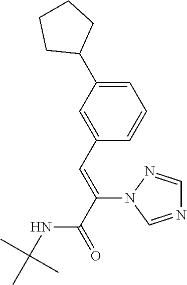 [SMILES: CC(C)(C)NC(=O)/C(=C/c1cccc(C2CCCC2)c1)n1cncn1] |
| 141 | 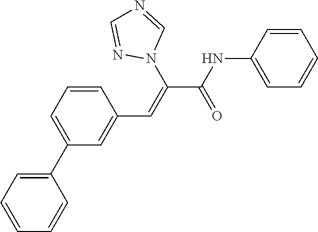 [SMILES: O=C(Nc1ccccc1)/C(=C/c1cccc(-c2ccccc2)c1)n1cncn1] |
| 142 | 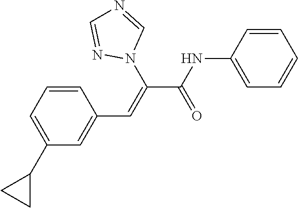 [SMILES: O=C(Nc1ccccc1)/C(=C/c1cccc(C2CC2)c1)n1cncn1] |
| 143 | 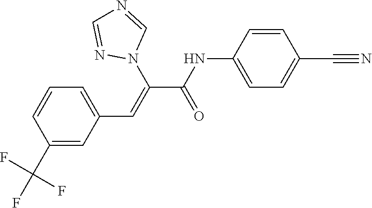 [SMILES: N#Cc1ccc(NC(=O)/C(=C/c2cccc(C(F)(F)F)c2)n2cncn2)cc1] |
| 144 | 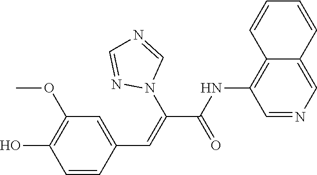 [SMILES: COc1cc(/C=C(/C(=O)Nc2cncc3ccccc23)n2cncn2)ccc1O] |
| 145 | 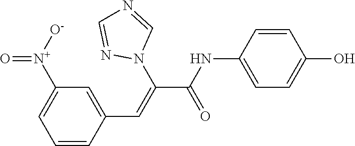 [SMILES: O=C(Nc1ccc(O)cc1)/C(=C/c1cccc([N+](=O)[O-])c1)n1cncn1] |
| 146 | 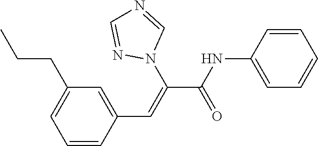 [SMILES: CCCc1cccc(/C=C(/C(=O)Nc2ccccc2)n2cncn2)c1] |
| 147 | 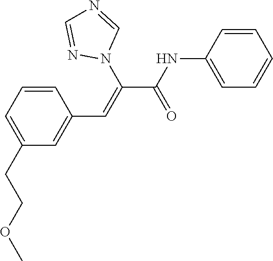 [SMILES: COCCc1cccc(/C=C(/C(=O)Nc2ccccc2)n2cncn2)c1] |
| 148 | 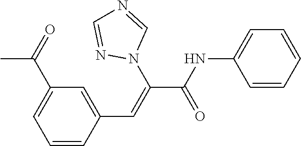 [SMILES: CC(=O)c1cccc(/C=C(/C(=O)Nc2ccccc2)n2cncn2)c1] |
| 149 | 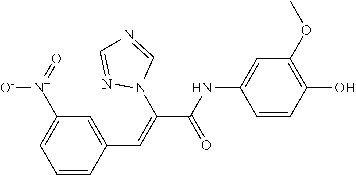 [SMILES: COc1cc(NC(=O)/C(=C/c2cccc([N+](=O)[O-])c2)n2cncn2)ccc1O] |
| 150 | 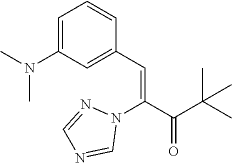 [SMILES: CN(C)c1cccc(/C=C(/C(=O)C(C)(C)C)n2cncn2)c1] |
| 151 | 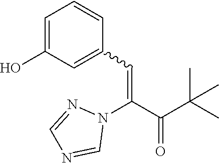 [SMILES: CC(C)(C)C(=O)C(=Cc1cccc(O)c1)n1cncn1] |
| 152 | 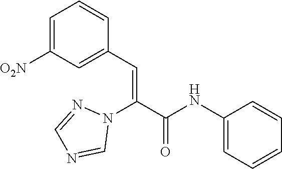 [SMILES: O=C(Nc1ccccc1)/C(=C/c1cccc([N+](=O)[O-])c1)n1cncn1] |
| 153 | 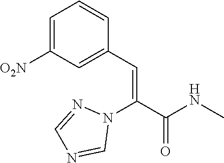 [SMILES: CNC(=O)/C(=C/c1cccc([N+](=O)[O-])c1)n1cncn1] |
| 154 | 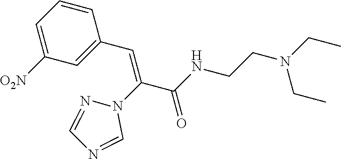 [SMILES: CCN(CC)CCNC(=O)/C(=C/c1cccc([N+](=O)[O-])c1)n1cncn1] |

11. The compound or pharmaceutically acceptable salt thereof according to claim 10, wherein the compound is selected from the following table:

| | |
|---|---|
| 150 | 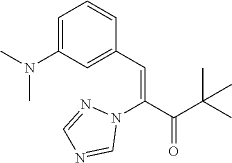 [SMILES: CN(C)c1cccc(/C=C(/C(=O)C(C)(C)C)n2cncn2)c1] |
| 151 | 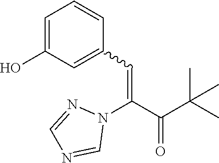 [SMILES: CC(C)(C)C(=O)C(=Cc1cccc(O)c1)n1cncn1] |
| 152 | 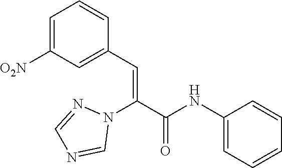 [SMILES: O=C(Nc1ccccc1)/C(=C/c1cccc([N+](=O)[O-])c1)n1cncn1] |
| 153 | 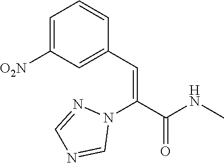 [SMILES: CNC(=O)/C(=C/c1cccc([N+](=O)[O-])c1)n1cncn1] |
| 154 | 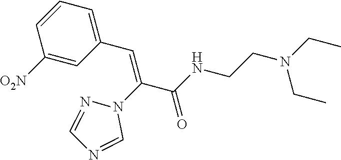 [SMILES: CCN(CC)CCNC(=O)/C(=C/c1cccc([N+](=O)[O-])c1)n1cncn1] |

12. A pharmaceutical composition comprising a compound or a pharmaceutically acceptable salt thereof according to any of claims 1 to 11 and a pharmaceutically acceptable carrier.

13. A method for selectively inhibiting exocytosis of granular proteins in neutrophils in a subject in need thereof, comprising administering to the subject a compound or a pharmaceutically acceptable salt thereof according to any of claims 1 to 11.

14. A method for treating a disease in a subject suffering therefrom, comprising administering to the subject a compound or a pharmaceutically acceptable salt thereof according to any of claims 1 to 11, wherein the disease etiology derives from neutrophil-mediated inflammation, upregulation of activation signature molecules in neutrophils, or increased tissue infiltration by inflammatory neutrophils.

15. The method according to claim 14, wherein the disease is an inflammatory disease or disorder.

16. The method according to claim 15, wherein the inflammatory disease is selected from Coronary Artery Disease, Inflammatory Arthritis, Ischemia-Reperfusion Injury, Autoimmune Disease, Sepsis, Infectious colitis, Ulcerative colitis, Crohn's disease, Ischemic colitis, Radiation colitis, Peptic ulcer, Intestinal cancer, Intestinal obstruction, Rheumatoid arthritis, Psoriatic arthritis, Hashimoto thyroiditis, Systemic lupus erythematosus, Multiple Sclerosis, Graves' Disease, Type 1 Diabetes Mellitus, Psoriasis, Ankylosing spondylitis, Scleroderma, Myositis, Gout, Antiphospholipid Antibody Syndrome (APS), Vasculitis, Dilated cardiomyopathy, Hypertrophic cardiomyopathy, Restrictive cardiomyopathy, Left-sided heart failure, Right-sided heart failure, Systolic heart failure, Diastolic heart failure (heart failure with preserved ejection fraction), Atrial Septal Defect, Atrioventricular Septal Defect, Coarctation of the Aorta, Double-outlet Right Ventricle, d-Transposition of the Great Arteries, Ebstein Anomaly, Hypoplastic Left Heart Syndrome, Interrupted Aortic Arch, Pulmonary Atresia, Single Ventricle, Tetralogy of Fallot, Total Anomalous Pulmonary Venous Return, Tricuspid Atresia, Truncus Arteriosus, Ventricular Septal Defect, Polycystic kidney disease, Diabetes Insipidus, Goodpasture's Disease, IgA Vasculitis, IgA Nephropathy, Lupus Nephritis, Adult Nephrotic Syndrome, Childhood Nephrotic Syndrome, Hemolytic Uremic Syndrome, Medullary Sponge Kidney, Kidney dysplasia, Renal artery stenosis, Renovascular hypertension, Renal tubular acidosis, Alport syndrome, Wenger's granulomatosis, Alagille syndrome, Cystinosis, Fabry disease, Focal segmental glomerulosclerosis (FSGS), Glomerulonephritis, aHUS (atypical hemolytic uremic syndrome), Hemolytic uremic syndrome (HUS), Henoch-Schonlein purpura, IgA nephropathy (Berger's disease), Interstitial nephritis, Minimal change disease, Nephrotic syndrome, Thrombotic thrombocytopenic purpura (TTP), Granulomatosis with polyangiitis (GPA), Eczema, Psoriasis, Cellulitis, Impetigo, Atopic dermatitis, Epidermolysis Bullosa, Lichen Sclerosis, Ichthyosis, Vitiligo, Acral peeling skin syndrome, Blau syndrome, Primary cutaneous amyloidosis, Cutaneous abscess, Pressure Ulcers, Blepharitis, Furunculosis, Full or partial thickness burns, Capillaritis, Cellulitis, Corneal Abrasion, Corneal Erosion, Xerosis, Lichen Planus, Lichen Simplex Chronicus, Venous Ulcer (Stasis Ulcer), Adult Still's disease, Agammaglobulinemia, Alopecia areata, Autoimmune angioedema, Autoimmune dysautonomia, Autoimmune encephalomyelitis, Autoimmune hepatitis, Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Bullous pemphigoid, Celiac disease, Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Coxsackie myocarditis, CREST syndrome, Dermatitis herpetiformis, Dermatomyositis, Devic's disease (neuromyelitis optica), Discoid lupus, Eosinophilic esophagitis (EoE), Eosinophilic fasciitis, Erythema nodosum, Essential mixed cryoglobulinemia, Giant cell arteritis (temporal arteritis), Giant cell myocarditis, Granulomatosis with Polyangiitis, Guillain-Barre syndrome, Hashimoto's thyroiditis, Henoch-Schonlein purpura (HSP), Herpes gestationis or pemphigoid gestationis (PG), Hypogammalglobulinemia, IgG4-related sclerosing disease, Immune thrombocytopenic purpura (ITP), Inclusion body myositis (IBM), Lambert-Eaton syndrome, Leukocytoclastic vasculitis, Linear IgA disease (LAD), Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Primary biliary cirrhosis, Primary sclerosing cholangitis, Progesterone dermatitis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Alagille Syndrome, Alcohol-Related Liver Disease, Autoimmune Hepatitis, Biliary Atresia, Cirrhosis, Lysosomal Acid Lipase Deficiency (LAL-D), Lysosomal storage disorders, Liver Cysts, Liver Cancer, Newborn Jaundice, Non-Alcoholic Fatty Liver Disease, Non-Alcoholic Steatohepatitis, Primary Biliary Cholangitis (PBC), Progressive Familial Intrahepatic Cholestasis (PFIC), Osteoporosis, Paget's Disease, Osteonecrosis, Osteoarthritis, Low Bone Density, Gout, Fibrous Dysplasia, Marfan Syndrome, Acute lung injury, acute respiratory distress syndrome, and Osteogenesis Imperfecta.
